(19) United States
(12) Patent Application Publication
(10) Pub. No.: US 2024/0002957 A1
(43) Pub. Date: Jan. 4, 2024

(54) DEVICE, SYSTEMS, KITS AND METHODS FOR RAPID AND SIMPLE DETECTION OF PATHOGENS

(71) Applicant: Technion Research and Development Foundation Ltd., Haifa (IL)

(72) Inventors: Naama GEVA-ZATORSKY, Nahalal (IL); Nadav BEN-ASSA, Ramat Hasharon (IL); Rawi NADDAF, Nazareth (IL); Tal GEFEN, Karmiel (IL)

(21) Appl. No.: 17/998,720

(22) PCT Filed: May 13, 2021
(86) PCT No.: PCT/IL2021/050557

§ 371 Date: Nov. 14, 2022

Related U.S. Application Data
(60) Provisional application No. 63/024,714, filed on May 14, 2020.

(51) Int. Cl. C12Q 1/70; B01L 7/00; B01L 3/00

(52) U.S. Cl. CPC C12Q 1/701; B01L 7/52; B01L 3/527; B01L 2400/0481; B01L 2300/043; B01L 2400/0611

(57) ABSTRACT

The present disclosure provides devices, systems, kits and methods for rapid, simple self-sampling and self-performing detection of nucleic acid sequence of interest in a sample, specifically, nucleic acid sequences of pathogenic agents.

### FIELD OF THE INVENTION

This invention relates to the field of detection of any nucleic acid sequence of interest, for example, any nucleic acid sequence of at least one pathogen, specifically, viral pathogen/s. More specifically, the invention provides devices, systems, kits and methods for rapid, simple self-sampling and self-performing detection of such pathogenic agents.

### BACKGROUND ART

References considered to be relevant as background to the presently disclosed subject matter are listed below:
[1] Lassauniere, R. et al. Evaluation of nine commercial SARS-CoV-2 immunoassays. medRxiv, 2020. Doi:10.1101/2020.04.09.20056325 (2020).
[2] Tang, Y.-W., Schmitz, J. E., Persing, D. H. & Stratton, C. W. The Laboratory Diagnosis of COVID-19 Infection: Current Issues and Challenges. Journal of Clinical Microbiology, JCM.00512-00520, doi:10.1128/jcm.00512-20 (2020).
[3] Bruce, E. A. et al. DIRECT RT-qPCR DETECTION OF SARS-CoV-2 RNA FROM PATIENT NASOPHARYNGEAL SWABS WITHOUT AN RNA EXTRACTION STEP. bioRxiv, 2020.2003.2020.001008, doi:10.1101/2020.03.20.001008 (2020).
[4] Zhang, Y. et al. Rapid Molecular Detection of SARS-CoV-2 (COVID-19) Virus RNA Using Colorimetric LAMP. medRxiv, 2020.2002.2026.20028373, doi:10.1101/2020.02.26.20028373 (2020).
[5] Notomi, T. et al. Loop-mediated isothermal amplification of DNA. Nucleic Acids Research 28, e63-e63, doi:10.1093/nar/28.12.e63 (2000).
[6] Yu, L. et al. Rapid colorimetric detection of COVID-19 coronavirus using a reverse tran-scriptional loop-mediated isothermal amplification (RT-LAMP) diagnostic plat-form: iLACO. medRxiv, 2020.2002.2020.20025874, doi:10.1101/2020.02.20.20025874 (2020).
[7] Wang, X. et al. Rapid and sensitive detection of Zika virus by reverse transcription loop-mediated isothermal amplification. Journal of Virological Methods 238, 86-93, doi:https://doi.org/10.1016/j.jviromet.2016.10.010 (2016).
[8] Ben Assa, N., et al., Direct on-the-spot detection of SARS-CoV-2 in patients. Experimental Biology and Medicine, 245, 1187-1193, (2020).
[9] Meselson, M. Droplets and Aerosols in the Transmission of SARS-CoV-2. New England Journal of Medicine, doi:10.1056/NEJMc2009324 (2020).
[10] Zheng, S. et al. Saliva as a Diagnostic Specimen for SARS-CoV-2 by a PCR-Based Assay: A Diagnostic Validity Study, The Lancet, doi:org/10.2139/ssrn.3543605 (2020).
[11] Curtis, K. A. et al. A multiplexed RT-LAMP assay for detection of group M HIV-1 in plasma or whole blood. Journal of Virological Methods 255, 91-97 (2018).

Acknowledgement of the above references herein is not to be inferred as meaning that these are in any way relevant to the patentability of the presently disclosed subject matter.

### BACKGROUND OF THE INVENTION

During a pandemic, surveillance is crucial for minimizing viral spread. The common and approved detection method worldwide requires professional experience in sampling, performing the reaction and analyzing the results. Moreover, it requires dedicated machines, and chemical reagents as well as sophisticated sample collection and transport logistics. Due to these laborious and cumbersome requirements, the number of detection tests per day is limited, and many patients in the community are not sampled, let alone sampled frequently. Such limited surveillance necessitates global and strict quarantine requirements, which threaten the global economy.

Detection is key. A simple and easy detection method, preferably one that can be performed and interpreted on-the-spot could relieve some of the current limitations and help execute an efficient and safe exit strategy from lockdowns. Fast and simple serological tests for SARS-CoV2 that can, in principle, be applied in households, are being developed [1]. However, anti-viral antibodies can be detected only several days after the infection onset and can persist even after clearance of the virus. A stage at which the patient is not contagious anymore [2]. Hence, the presence of antibodies detected in such home-kits are an indirect indication on previous viral exposure. These tests do not account to the actual viral load, a critical parameter for minimizing the spread.

Detection of viral nucleic acids in patients is the gold standard detection method to date. It is currently performed at hospitals by professionals. As opposed to antibodies, detection of the viral RNA is a direct measure for the contagiousness of the patient. At this stage of the current COVID-19 pandemic, it is clear that the availability and throughput of standard methods for viral nucleic acid detection is limited both by resources and accessibility to the community.

Standard detection methods for viral RNA in patients include RNA purification, reverse transcription and quantitative PCR (RT-qPCR). These processes are time consuming, require multiple biochemical reagents, lab-grade instruments and trained professionals [3]. Fortunately to date, alternative molecular biology methods can overcome these limitations. One of these methods is colorimetric Loop-Mediated Isothermal Amplification (LAMP) [4]. LAMP is performed at a single and constant temperature allows a one-step reverse transcription and its results can be visualized by color change. Due to the need for reverse transcription, this method is called reverse-transcribed (RT)-LAMP. These advantages reduce the need for sophisticated lab equipment [5-7]. The inventors recently established a direct method for detecting SARS CoV-2 in samples [8]. There is a need for simple and rapid methods to provide results on-the-spot to detect viral nucleic acid materials from crude subject's samples in order to allow continuous surveillance of a community.

### SUMMARY OF THE INVENTION

A first aspect of the invention relates to a device for enabling detection of at least one nucleic acid sequence of interest in at least one sample, comprising a sample module and a reaction module.

More specifically, the sample module of the device of the invention is configured for accommodating therein a plurality of sample preparation agents including at least a sample to be tested, and for enabling mixing of said plurality of sample preparation agents to provide a prepared sample. It should be noted that the sample module being configured for selectively allowing insertion of the sample into the sample module, and further configured for enabling delivery of said prepared sample to the reaction module.

Still further, the reaction module of the device of the invention comprises a plurality of reaction chambers. More specifically, the device configured for selectively delivering a respective aliquot of said prepared sample to each reaction chamber. It should be noted that each said reaction chamber is configured for accommodating therein a respective third quantity of a respective reaction mixture adapted for amplification reaction under isothermal conditions.

The device of the invention is further configured for enabling reacting of each said respective aliquot of said prepared sample with each said respective reaction mixture in the respective said reaction chamber to produce at least one amplification product.

The device is further configured for enabling detecting a respective test parameter associated with said production of said at least one amplification product. In some embodiments, the sample preparation agents may comprise at least one protease. Thus, the sample module of the invention may comprise in some embodiments, a first quantity of at least one protease, and a second quantity of a solubilizing liquid. In certain embodiments, at least one of the sample module and the reaction module of the device of the invention further comprise a fourth quantity of at least one chaotropic agent.

It should be noted that in some embodiments, the device of the invention may be applicable for detecting any nucleic acid sequence of interest, for example, any nucleic acid sequence of at least one pathogen, such as a viral pathogen. In more specific and non-limiting embodiments, the device of the invention is applicable for detecting Severe acute respiratory syndrome (SARS) corona virus 2 (SARS-CoV2) in a sample.

A further aspect of the invention relates to a system for detecting at least one nucleic acid sequence of interest, for example, any nucleic acid sequence of at least one pathogen in at least one sample.

The system of the invention comprises at least one devise that comprise at least one sample module and at least one reaction module, specifically, any of the devices disclosed by the invention and a heating apparatus configured for heating at least the reaction module to a predetermined range of temperatures above ambient.

A further aspect of the invention relates to a method for the detection and monitoring of at last one nucleic acid sequence of interest, for example, any nucleic acid sequence of at least one pathogen in at least one sample. More specifically, in some embodiments the methods of the invention may comprise the following steps: First step (a), involves contacting the sample or at least one aliquot thereof with an effective amount of at least one sample preparation agent to obtain at least one prepared sample.

The next step (b) involves subjecting the at least one prepared sample of (a), or at least one aliquot thereof to at least one amplification reaction under isothermal conditions suitable for the production of at least one amplification product detectable by a detectable signal, that is further referred to herein as a test parameter. It should be noted that at least one of such amplification reaction/s may be performed using at least one set of primers specific for at least one nucleic acid sequence of the nucleic acid sequence of interest, for example, any nucleic acid sequence of at least one pathogen. In some embodiments, the sample preparation agent/s may comprise at least one nucleic acid stripping compound. In some embodiments, such compound may be at least one protease.

It should be noted that in some embodiments, the method of the invention may further comprise in at least one of steps (a) and (b), contacting the sample with at least one chaotropic agent. More specifically, the at least one cheotropic agent may be contacted with the sample in step (a), during the preparation and incubation with the at least one protease. Alternatively, the at least one cheotropic agent may be contacted with the examined sample during the amplification reaction. In yet some further embodiments, the at least one cheotropic agent may be added to both steps, during the preparation of the sample in step (a) and during the amplification reaction in step (b).

It should be noted that a detection of a detectable signal indicates the presence of the nucleic acid sequence of interest, for example, any nucleic acid sequence of at least one pathogen in the examined sample.

In yet a further aspect thereof, the invention provides a kit comprising: First, as a component (a), the kit of the invention comprises an effective amount of at least one sample preparation agent/s, for example, at least one protease. The protease may be provided in any buffer or solution, either in liquid or in a solid form, for example, as a lyophilized preparation.

In some optional embodiments, the at least one protease of the kit of the invention may be provided comprised within a sample preparation module. In more specific embodiments, such sample module may be any of the sample modules disclosed by the invention in connection with any of the devices or any of the systems of the invention.

The kit of the invention may comprise as a component (b), an amplification reaction mixture. In some embodiments, the reaction mixture may comprise any reagent, solution and/or component required for performing and/or improving an amplification reaction. Specifically, any amplification reaction under isothermal conditions. Specific examples for reaction mixtures useful in the kits of the invention are indicated in more detail herein after. In some embodiments, the reaction mixtures provided by the kits of the invention may be comprised within a reaction module.

In more specific embodiments, such reaction module may comprise a plurality of reaction chambers, each of such reaction chambers comprises the amplification mixture. As indicated above, the reaction mixture is adapted for amplification reaction under isothermal conditions. In more specific embodiments, the reaction mixture in at least one first reaction chamber may comprise at least one set of primers specific for at least one nucleic acid sequence of at least one nucleic acid sequence of interest, for example, any nucleic acid sequence of at least one pathogen.

It should be understood that in some embodiments, the reaction buffer can be provided within at least one reaction chamber. In yet some further embodiments, the at least one reaction chambers may be comprised within the reaction module of the invention.

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1H: Protocol adjustment and optimal conditions.

FIG. 1A: Schematic representation of the isothermal, colorimetric RT-LAMP reaction.

FIG. 1B: RT-LAMP reaction on purified RNA from nasal and throat swabs submerged in UTM buffer. Results shown at t=0 and after 30 minutes incubation at 65° C. (left) No template control (NTC), (middle) negative subject (Neg S.) and (right) positive subject (Pos S.). Three technical replicates of each sample are shown.

FIG. 1C: Representative pictures of RT-LAMP test results of crude nasal and throat swabs. Samples were directly tested with no RNA purification step. (left) three different negative samples (Neg S.), and (right) three different positive samples (Pos S.) at t=0 and t=30 minutes.

FIG. 1D: Comparison of the RT-LAMP method to the Ct values of the standard RT-qPCR results (3 true positive and 2 false negative samples out of the 99 that were analyzed, are not shown due to inaccessibility to their RT-qPCR Ct values). RT-qPCR negative samples were assigned arbitrary Ct values, for visualization.

FIG. 1E: Classification of true positive (TP), true negative (TN), false positive (FP) and false negative (FN) numbers and rate of RT-LAMP test results in comparison to the standard RT-qPCR test results.

FIG. 1F: Crude nasal and throat swabs tested by two different RT-LAMP protocols. Upper panel, without proteinase K and guanidine hydrochloride. Lower panel, with proteinase K treatment and guanidine hydrochloride. For RT-qPCR positive samples, Ct value is presented under each sample, the sample to the right is negative.

FIG. 1G: Specificity of RT-LAMP detection method for SARS-CoV-2. RT-LAMP results on different samples from patients confirmed to be positive for the following virus: 1-2, HSV swabs (lysed and inactivated as described in the currently developed protocol). 3, HSV purified DNA. 4, RSV purified RNA. 5, Influenza B RNA. 6, Enterovirus RNA. 7, RNA extraction from SARS-CoV-2 positive patient. 8, no template control. Results are shown at t=0 and t=30 minutes after incubation at 65° C.

FIG. 1H: Representative pictures of individual colored samples. Shown at t=0, t=30 and t=40 minutes of the RT-LAMP reaction. Numbers above and under each of the tubes relate to the number of the sample in Table 2.

FIG. 2A-2E: Adjusted RT-LAMP protocol tested on 83 crude nasal and throat samples.

FIG. 2A: Classification of true positive (TP), true negative (TN), false positive (FP) and false negative (FN) numbers and rates of RT-LAMP test results in comparison to the standard RT-qPCR results. Boxes from left to right represent results at t=30, 35 and 40 minutes, respectively.

FIG. 2B: bar graph representation of the TP, TN, FP and FN rates shown in (a).

FIG. 2C: Comparison of the RT-LAMP method to the Ct values of the standard RT-qPCR.

FIG. 2D: Graphical representation of TP rates of RT-LAMP in different incubation periods, t=30 min, t=35 min, t=40 min, compared to RT-qPCR test results separated by Ct value intervals. 29<Ct<35 (left), 26<Ct<29, (middle), Ct<26 (right).

FIG. 2E: Representative pictures of individual colored samples. Shown at t=0, t=30, t=35 and t=40 minutes of the RT-LAMP reaction. Numbers above and under each of the tubes relate to the number of the sample in Table 3.

FIG. 3A-3B: Applying the RT-LAMP protocol on saliva samples.

FIG. 3A: RT-LAMP tests on saliva from 4 volunteers. Each tube represents one tested volunteer. Results of t=0 and t=35 are shown. Upper panel, RT-LAMP reaction using POP7 primers as a positive control. Middle panel, RT-LAMP reaction with no primers control. Lower panel, RT-LAMP reaction using SARS-CoV-2 gene N primers. The same samples were analyzed by the conventional hospital RT-qPCR protocol. The RT-qPCR results and Ct values are shown under the relevant samples.

FIG. 3B: Graphical illustration of the potential of RT-LAMP protocol to perform self-saliva testing.

FIG. 4 The device.

The figure shows a cross-sectional side view of a device for enabling detection of at least one pathogen in at least one sample according to a first example of the presently disclosed subject matter.

FIG. 5 The system.

The figure shows a schematic illustration of a system for a detection of at least one nucleic acid sequence of interest, for example, of at least one pathogen in at least one sample according to a first example of the presently disclosed subject matter.

FIG. 6A-6C. The device

FIG. 6A. is a schematic illustration of a device for enabling detection of at least one nucleic acid sequence of interest, for example, of at least one pathogen in at least one sample according to a second example of the presently disclosed subject matter, prior to insertion of a sample cassette/cartridge; FIG. 6B is a schematic illustration of the device of FIG. 6A wherein the sample preparation agents are mixed together; FIG. 6C is a schematic illustration of the device of FIG. 6A and FIG. 6B, wherein the prepared sample is inserted into the reaction module and reaction completed in the reaction chambers.

FIG. 7A-7E. The device, system and process.

FIGS. 7A to 7E, show a schematic illustration of a device and system for enabling detection of at least one nucleic acid sequence of interest, for example, of at least one pathogen in at least one sample according to a third example of the presently disclosed subject matter, in various stages of use.

FIG. 8A-8D. The device, system and process.

FIGS. 8A to 8D, show a schematic illustration of a device and system for enabling detection of at least one pathogen in at least one sample according to a fourth example of the presently disclosed subject matter, in various stages of use.

### DETAILED DESCRIPTION OF THE INVENTION

During the COVID-19 pandemic, many countries were in a lockdown state. One key aspect to transition safely out of lockdown is to continuously test the population for infected subjects. Currently, detection is performed at points of care using quantitative reverse-transcription PCR (RT-qPCR) and requires dedicated professionals and equipment. Here, a protocol was developed based on Reverse Transcribed Loop-Mediated Isothermal Amplification (RT-LAMP) for detection of SARS-CoV-2, directly from crude nose and throat swabs.

The experiments were performed side-by-side to the standard RT-qPCR method at the hospital. RT-qPCR Ct values were compared to the RT-LAMP results of more than 180 different patients. This enabled to determine the RT-LAMP specificity and limit of detection from crude patient swabs. Upon calibration, this direct RT-LAMP method successfully detected patients with medium to high viral loads, while yielding very few false-positives.

The optimal protocol was demonstrated for immediate off-the-shelf use of RT-LAMP on COVID-19 patients in the current pandemic. This protocol does not include RNA purification step. Besides a constant heat source, (e.g., a thermal mug), no sophisticated equipment is required. This protocol takes about an hour from sampling to detection, with very few reagents, and can be performed by non-professionals or self-performed. These features allow its implementation around the globe, including in rural areas.

Upon clinical approval, this SARS-CoV-2 detection method can be applied as a surveillance tool for sampling larger populations of the community, for example, in medical clinics. Its simplicity, availability of products and low cost, makes it easy to continuously monitor suspected subjects. With further development, this method can be applied to points of entry, workplaces and even at home. Importantly, this method can be easily adjusted to any nucleic acid sequence of interest, for example, of at least one pathogen, such as the SARS CoV-2, or any other emerging pathogens as well.

Thus, in a first aspect, the present disclosure provides a device for enabling detection of at least one nucleic acid sequence of interest in at least one sample, comprising a sample module and a reaction module. More specifically, wherein:
the sample module is configured for accommodating therein a plurality of sample preparation agents including at least a sample to be tested, and for enabling mixing of said plurality of sample preparation agents to provide a prepared sample, said sample module being configured for selectively allowing insertion of the sample into the sample module, and further configured for enabling delivery of said prepared sample to the reaction module;
the reaction module comprises a plurality of reaction chambers, the device configured for selectively delivering a respective aliquot of said prepared sample to each said reaction chamber, wherein each said reaction chamber is configured for accommodating therein a respective quantity of a respective reaction mixture adapted for amplification reaction under isothermal conditions;
the device is further configured for enabling reacting of each said respective aliquot of said prepared sample with each said respective reaction mixture in the respective said reaction chamber to produce at least one amplification product; and
the device is further configured for enabling detecting a respective test parameter associated with said production of said at least one amplification product.

In some embodiments of the device of the present disclosure, at least one of:
(a) the sample preparation agents comprise a first quantity of at least one protease, and a second quantity of a solubilizing liquid;
(b) the respective quantity of a respective reaction mixture is a respective third quantity of a respective reaction mixture adapted for amplification reaction under isothermal conditions; and
(c) at least one of the sample module and said reaction module further comprise a fourth quantity of at least one chaotropic agent.

In some embodiments, the sample module of the device of the present disclosure comprises a sample module housing defining therein a sample chamber, the sample chamber being configured for accommodating therein the plurality of sample preparation agents and for enabling mixing of the plurality of sample preparation agents to provide said prepared sample.

In yet some further embodiments, the sample module is configured for accommodating said first quantity of at least one protease therein, prior to use of the device with a user.

Still further, in some embodiments, the sample module of the device of the present disclosure is configured for enabling the second quantity of a solubilizing liquid to be selectively inserted into the sample chamber from an external source.

In some embodiments, the sample chamber of the device provided herein is configured for separately accommodating therein the plurality of sample preparation agents and for enabling selectively mixing of said plurality of sample preparation agents to provide the prepared sample.

In yet some further embodiments, the sample module of the device disclosed herein comprises a first sub-chamber configured for accommodating said first quantity of at least one protease therein, prior to use of the device with a user, and a second sub-chamber, different from said first sub-chamber, wherein the second sub-chamber is configured for accommodating said second quantity of a solubilizing liquid therein, prior to use of the device with a user, and wherein said sample module comprises a third sub-chamber configured for accommodating the sample when the device is used with respect to a user.

In some embodiments, the sample module is configured for selectively enabling fluid communication between said first sub-chamber, the second sub-chamber, and the third sub-chamber to enable mixing of said plurality of sample preparation agents to provide the prepared sample.

In some embodiments, the sample module of the device of the present disclosure comprises a membrane separating the second sub-chamber from said first sub-chamber and said second sub-chamber. Still further, the membrane is selectively rupturable to enable mixing of the plurality of sample preparation agents to provide the prepared sample.

In yet some further embodiments, the sample module of the device disclosed herein comprises a barrier separating the second sub-chamber from the first sub-chamber and the second sub-chamber, and the barrier comprises a first valve member that is selectively openable to enable mixing of said plurality of sample preparation agents to provide the prepared sample.

In yet some further embodiments, the sample module of the device of the present disclosure comprises an inlet port for selectively allowing insertion of at least the sample therethrough and into the sample chamber.

Still further, in some embodiments, the sample module comprises a cap for selectively opening and closing said inlet port, and wherein the cap includes a sampling member projecting from an inner part of the cap such that when the cap closes the inlet port the sampling member is inside the sample chamber.

In yet some further embodiments, the sample module of the device of the present disclosure may comprise a sampling member in the form of a cartridge that is selectively insertable into the sample chamber via the inlet port. Moreover, the cartridge includes a sample surface onto which a user can deposit the sample thereon.

In certain embodiments, the sample module includes a sample surface spaced from the inlet port. Still further, the sample module is configured for enabling a user to deposit the sample onto the sample surface via the inlet port.

In yet some further embodiments, the sample module of the is configured for enabling delivery of said prepared sample to the reaction module from the sample chamber.

Still further, in some embodiments, the sample chamber of the device disclosed herein comprises an outlet port coupled to the reaction module, the outlet port being configured for selectively enabling fluid communication between the sample chamber and the reaction module to thereby enable delivery of the prepared sample to the reaction module from the sample chamber. In yet some further optional embodiments, the outlet port comprising a second valve member configured for selectively enabling fluid communication between the sample chamber and the reaction module to thereby enable delivery of said prepared sample to the reaction module from the sample chamber.

Still further, in some embodiments, the device of the present disclosure is configured for selectively separating the prepared sample into a plurality the aliquots of the prepared sample, and for directing each said aliquot to a respective the reaction chamber. In yet some further embodiments, the reaction module comprises a manifold unit having an inlet opening in fluid communication with the outlet port, and a plurality of exit ports, each exit port being in fluid communication with a respective the reaction chamber.

In some embodiments, the reaction chamber of the device disclosed herein is configured for accommodating therein said respective third quantity of the respective reaction mixture, prior to use of the device with a user.

Still further, in some embodiments, the reaction module of the device disclosed herein comprises at least two said reaction chambers, wherein a first said reaction chamber is configured for accommodating therein said respective third quantity of a respective first said reaction mixture, and wherein a second said reaction chamber is configured for accommodating therein a said respective third quantity of a respective second said reaction mixture, prior to use of the device with a user. In yet some further embodiments, optionally, the first reaction mixture is configured for testing for production of at least one amplification product of a nucleic acid sequence of the pathogen, and wherein said second reaction mixture is configured for production of at least one amplification product of a control nucleic acid sequence, thereby providing a control test.

In some embodiments of the device disclosed herein, each said respective test parameter is in the form of a visually detectable specific color associated with respective reaction mixture subsequent to interaction of the respective aliquot of the prepared sample with the respective the reaction mixture in the respective said reaction chamber. In certain optional embodiments, at least a part of the reaction module is transparent to allow each the specific color to be externally observed, recorded and/or quantified.

Still further, in some embodiments of the device disclosed herein, each of the respective test parameter is detectable as any one of a specific fluorescence parameter, a specific pH value, electric charge and conductivity, or production of pyrophosphate each being associated with respective reaction mixture subsequent to interaction of said respective aliquot of said prepared sample with the respective said reaction mixture in the respective said reaction chamber. In yet some further embodiments, the reaction module is configured for enabling, for each reaction chamber, the respective the specific fluorescence parameter, the respective said specific pH value, or the respective said electric charge, or the respective conductivity, or the respective production of pyrophosphate to be externally detected, recorded and/or quantified.

In certain embodiments, each said reaction chamber of the disclosed device, comprises therein the respective third quantity of the respective reaction mixture, and wherein said sample module comprises therein said first quantity of at least one protease, and wherein said device is configured for enabling selective delivery of said second quantity of a solubilizing liquid into said sample module. In yet some further embodiments, the device further comprising said fourth quantity of at least one chaotropic agent.

In some embodiments, the reaction chamber of the disclosed device comprises therein the respective third quantity of the respective reaction mixture, and wherein the sample module comprises therein said first quantity of at least one protease, and said second quantity of a solubilizing liquid, wherein said sample module is configured for initially maintaining the first quantity of at least one protease separate from the second quantity of a solubilizing liquid, and for selectively enabling mixing of the first quantity of at least one protease with the second quantity of a solubilizing liquid. In some optional embodiments, the device further comprising the fourth quantity of at least one chaotropic agent.

Still further, in some embodiments, at least one protease of said first quantity is proteinase K (PK). In yet some further embodiments, the solubilizing liquid of the second quantity comprises water. Still further embodiments of the device disclosed herein, the at least one chaotropic agent of said fourth quantity is guanidine hydrochloride.

In some further embodiments of the disclosed device, the amplification reaction is a loop mediated isothermal amplification reaction (LAMP). In yet some further embodiments, the at least one of the third quantity of the at least one first reaction mixture comprises at least one set of primers specific for at least one nucleic acid sequence of the at least one nucleic acid sequence of interest, optionally, at least one of said third quantity of the at least one second reaction mixture comprises at least one set of primers specific for at least one control nucleic acid sequence. In some further embodiments, the sample is at least one of a biological sample and an environmental sample.

In some further embodiments of the device of the present disclosure, the nucleic acid sequence of interest is a nucleic acid sequence of at least one pathogen. In some optional embodiments, the pathogen is a viral pathogen. In yet some further embodiments, the pathogen is a viral pathogen.

In some specific embodiments, the viral pathogen is at least one corona virus (CoV). Still further, in some embodiments, the pathogen is a CoV, wherein said CoV is Severe acute respiratory syndrome (SARS) CoV-2.

In a further aspect thereof, the present disclosure provides a system for detecting at least one nucleic acid sequence of interest, for example, of a pathogen in at least one sample. More specifically, the system disclosed herein may comprise at least one device as defined in the present disclosure, and a heating apparatus configured for heating at least the reaction module to a predetermined range of temperatures above ambient.

In some embodiments, the heating apparatus is configured for being coupled to the device and comprises a heating system configured for directing heat to the device when coupled thereto. In some embodiments the predetermined range of temperatures includes a temperature of 65° C.+/−5° C. In yet some further embodiments, the predetermined range of temperatures includes a temperature of 95° C.+/−5° C.

In some embodiments, the system disclosed herein further comprising detection apparatus configured for detecting said test parameters associated with each of the respective said reaction chambers. In some embodiments, the test parameter for each respective the test chamber is a respective said specific color, and wherein the detection apparatus is configured for determining a respective wavelength of the respective said specific color.

In yet some further embodiments, the test parameter for each respective the test chamber is a respective said specific fluorescence parameter, and wherein the detection apparatus is configured for determining a respective fluorescence value of the respective said specific fluorescence parameter. In some further embodiments, the test parameter for each respective said test chamber is a respective the specific pH parameter, and wherein the detection apparatus is configured for determining a respective pH value of the respective said specific pH parameter.

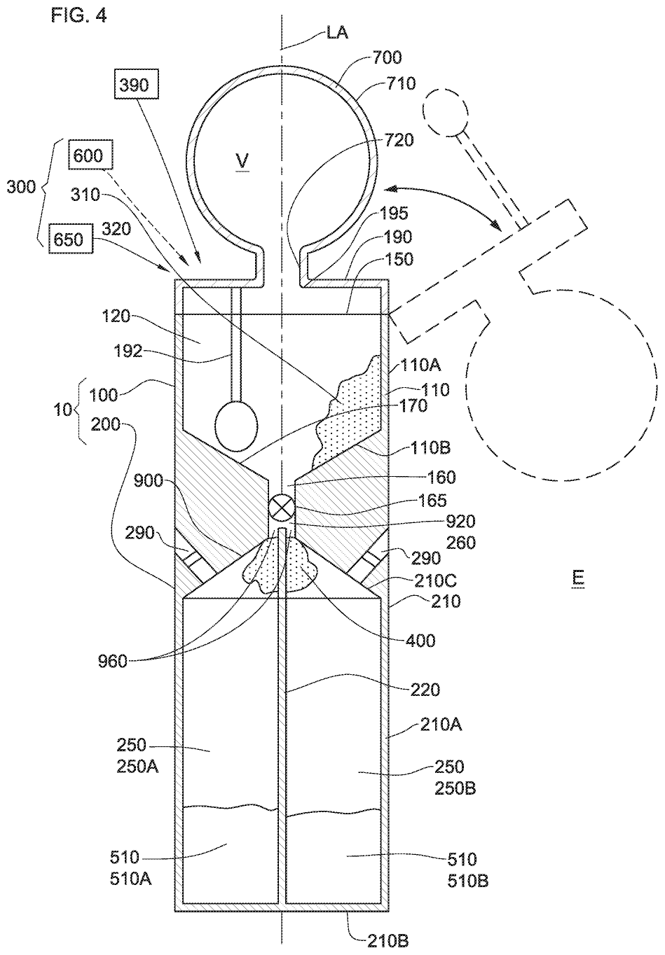

More specifically, referring to FIG. 4, a device for enabling detection of at least one pathogen in at least one sample according to a first example of the presently disclosed subject matter, generally designated 10, comprises a sample module 100 and a reaction module 200. As will become clearer below, according to an aspect of the presently disclosed subject matter the device 10 is part of a system 20 for detecting at least one nucleic acid sequence of interest, for example, of at least one pathogen in at least one sample.

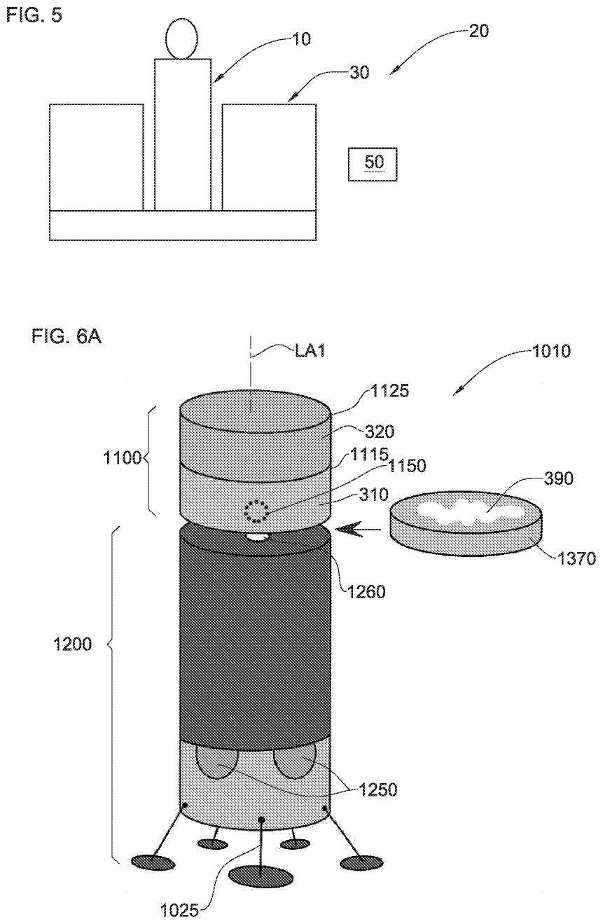

In at least this example, the sample module 100 and the reaction module 200 are integrated into a unitary article thereby facilitating operation thereof, in particular for detecting the at least one nucleic acid sequence of interest, for example, any nucleic acid sequence of at least one pathogen in the at least one sample when used as part of the system 20 (FIG. 5).

The sample module 100 is configured for accommodating therein a plurality of sample preparation agents 300 including at least a sample to be tested 390, a first quantity of at least one protease 310, and a second quantity of a solubilizing liquid 320, and for enabling mixing of the plurality of sample preparation agents 300 to provide a prepared sample 400.

The sample module 100 comprises a sample module housing 110 defining therein a sample chamber 120, the sample chamber 120 being configured for accommodating therein the plurality of sample preparation agents 300 and for enabling mixing of the plurality of sample preparation agents 300 including the sample 390 to provide the prepared sample 400.

The sample module housing 110 has, in at least this example, a general cylindrical wall 110A, connected to lower base wall 110B providing a cup-like structure enclosing therein a volume defining the sample chamber 120. For example, the cylindrical wall can have cross-sections that can be of any suitable shape, for example, circular, oval, elliptical, superelliptical, square, rectangular or polygonal having any number of sides.

In at least this example, and in other examples, the sample module 100 is configured for accommodating the first quantity of at least one sample preparation agent/s 310 therein, prior to use of the device 10 with a user. In some embodiments, the sample preparation agent/s may comprise at least one nucleic acid stripping compound. In some embodiments, such compound may be at least one protease. In other words, the sample module 100 comes with the first quantity of at least one protease 310 already accommodated in the sample chamber 120 ready for use by the user. It is to be noted that in alternative variations of this example, the first quantity of at least one sample preparation agent/s, for example, at least one protease 310 needs to be selectively inserted into the sample module 100 from a respective external source (designated with reference numeral 600, shown as phantom lines), for example in a similar manner to the selective insertion of the second quantity of a solubilizing liquid 320 to sample chamber 120 from another respective external source, when the device 100 is in the process of being used by the user.

In at least this example, and in other examples, the sample module 100 is configured for enabling said second quantity of a solubilizing liquid 320 to be selectively inserted into said sample chamber 110 from an external source 650, as will become clearer below.

The sample module 100 is further configured for selectively allowing insertion of the sample 390 into the sample module 100, and further configured for enabling delivery of the prepared sample 400 to the reaction module 200.

In at least this example, the sample module 100 comprises an inlet port 150 for selectively allowing insertion of at least the sample 390 therethrough and into the sample chamber 120.

Furthermore, in at least this example, the sample module comprises a cap 190 for selectively opening and closing the inlet port 150. The cap 190 is hingedly or detachably mounted to the sample module housing 110 at or close to the inlet port 150.

Optionally, the cap 190 includes a sampling member 192 in the form of a rod projecting from an inner part of the cap 190 such that when the cap closes the inlet port 150 the sampling member 192 is inside the sample chamber 120. For example, the sampling member 192 can include a loop 193 at a free end of the rod of the sampling member 192 for facilitating obtaining the sample 390 from any one of a nose, mouth and throat of a subject. In more specific embodiments, the loop is submerged into the saliva in the patients' mouth, collecting the saliva into the sample chamber.

In some alternative variations of the above examples, the sample module 100 can comprise a sampling member in the form of a cartridge or cassette that is selectively insertable into the sample chamber 120 via the inlet port 150, and wherein the cartridge or cassette includes a sample surface onto which a user can deposit the sample thereon. When engaged with the sample chamber 120, the sample surface of the cartridge or cassette is facing the internal volume of the sample chamber 120, and thus the sample 390 can interact and mix with the other sample preparation agents 300.

In at least this example, the sample module 100 includes a sample surface 170 spaced from said inlet port 150, and the sample module 100 is configured for enabling a user to deposit the sample 390 onto the sample surface 170—via the inlet port 150. In the illustrated example, the sample surface 170 is within the sample chamber 120 and generally opposite the inlet opening 150, such that opening the cap 190 exposes the sample surface 170 to the externa environment E outside of the device 10.

For example, the user can deliver the sample onto the sample surface 170 in any suitable manner, for example in case of saliva sample that may be captured using a loop, as discussed above. In some alternative embodiments, the saliva sample is placed by the user, that may either lick the surface of the sampler (sampler stick), or alternatively by using chewing gum, using a sponge, or any other absorbing matrix (paper, fabric and the like) or a gum collecting saliva from mouth or by washing the mouth and collecting a saliva sample after wash.

The device 10, and in particular the sample module 100, is further configured for enabling delivery of the prepared sample 400 to the reaction module 200 from the sample chamber 120. In at least this example the sample chamber 120 comprises an outlet port 160 coupled to the reaction module 200. The outlet port 160 is configured for selectively enabling fluid communication between the sample module 100 (in particular the sample chamber 120) and the reaction module 200 to thereby enable selective delivery of the prepared sample 400 to the reaction module 200 from the sample chamber 120.

In this connection, and in at least this example, the outlet port 160 comprises an outlet port valve member 165 configured for selectively enabling fluid communication between the sample chamber 120 and the reaction module 200 to thereby enable delivery of the prepared sample 400 to the reaction module 200 from the sample chamber 120. For example, the outlet port valve member 165 can be in the form of a one-way valve, for example duckbill valve (for example made from silicone), that allows fluid flow of the prepared sample in one direction to the reaction module 200 from the sample module 100, but not in the reverse direction from the reaction module 200 to the sample module 100. Alternatively, the outlet port valve member 165 can be in the form of a sliding door that slides transversely across the outlet port 160 to selectively close or open the outlet port 160.

While in this example, and in other examples, the reaction module 200 comprises two reaction chambers 250, it is to be noted that in yet other examples, the device can have a plurality of reaction chambers 250, including more than two reaction chambers 250.

The reaction module 200 comprises a reaction module housing 210, having, in at least this example, a general cylindrical wall 210A, connected at a lower end thereof to lower base wall 210B, and connected to an upper end thereof to upper base 210C having a reaction module inlet 260, enclosing therein a volume defining the plurality of reaction chambers 250, which are separated from one another via partition wall 220. In alternative variations of this example and in other examples in which there are more than three reaction chambers 250, these can be separated from one another via a suitable number of suitable partition walls. In yet other alternative variations of this example and in other examples, each reaction chamber can be provided in the form of a separate vessel within the reaction module housing 210, for example, each such vessel having a conduit for proving selective fluid communication between the respective reaction chamber 250 and the sample chamber 120 to thereby provide the require selective fluid communication between each reaction chamber 250 and the sample module 100.

Furthermore, in at least this example, the sample module 100 and the reaction module 200, in particular the sample module housing 110 and the reaction module housing 210 respectively, are co-axially aligned along the longitudinal axis LA of the device 10.

The device 10 is further configured for selectively delivering a respective aliquot of the prepared sample 400 to each reaction chamber 250, wherein each reaction chamber 250 is configured for accommodating therein a respective third quantity of a respective reaction mixture 510 adapted for amplification reaction under isothermal conditions.

In at least this example, and in other examples, each reaction chamber 250 is configured for accommodating therein said respective third quantity of said respective reaction mixture, prior to use of the device with a user. In other words, the reaction module 200 comes with the respective third quantity of the respective reaction mixture 510 already accommodated in the respective reaction chamber 250 ready for use by the user.

It is to be noted that at least in some examples, optionally including the above example, the reaction mixture can comprise a pH sensitive indicator dye to provide a detectable signal upon production of at least one amplification product. For example, the pH sensitive indicator dye is a colored dye detectable in visible light, or, the pH sensitive indicator dye is a fluorescent indicator dye. In yet some further alternative examples, the reaction mixture can comprise a fluorescent agent capable of intercalating into the amplification product.

In at least this example, one of the two reaction chambers, referred to herein as the first reaction chamber 250 and also designated herein with reference numeral 250A, is configured for accommodating therein a respective third quantity of a first reaction mixture 510, also designated herein with reference numeral 510A, prior to use of the device with a user. Furthermore, in this example, the other reaction chamber, referred to herein as the second reaction chamber 250 and also designated herein with reference numeral 250B, is configured for accommodating therein a respective third quantity of a different, second reaction mixture 510, also designated herein with reference numeral 510B, prior to use of the device with a user. In other words, the reaction module 200 comes with the respective third quantity of the first reaction mixture 510A already accommodated in the first reaction chamber 250A, and with the respective third quantity of the second reaction mixture 510B already accommodated in the second reaction chamber 250B, ready for use by the user.

In at least this example, the first reaction mixture 510A is configured for testing for production of at least one amplification product of a nucleic acid sequence of said nucleic acid sequence of interest, for example, any nucleic acid sequence of at least one pathogen, and the second reaction mixture 510B is configured for production of at least one amplification product of a control nucleic acid sequence, thereby providing a control test.

In at least this example, since there are two reaction chambers 250, i.e., first reaction chamber 250A and second reaction chamber 250B, each aliquot is approximately 50% of the quantity of prepared sample 400 that enters the reaction module 200 from the sample module 100.

The device 10 is further configured for selectively separating the prepared sample into a plurality the aforesaid aliquots of the prepared sample 400, and for directing each such aliquot to a respective said reaction chamber 250. For this purpose the reaction module can comprise a manifold unit 900, formed in the upper base 210C, and having an inlet opening 920 and a plurality of exit ports 960. The inlet opening, corresponding to or in fluid communication with the reaction module inlet 260, is in fluid communication with the outlet port 160 via valve member 165. Furthermore, each exit port 960 is in fluid communication with a respective reaction chamber 250. In at least this example, the partition wall 220 projects into the reaction module inlet 260, thereby providing two such exit ports 960. Thus it is anticipated that any as amount of the prepared sample 400, flowing into the reaction module inlet 260 from the outlet port 160, in particular from the outlet port valve member 165, is automatically split into a number of aliquots according to the number of exit ports 960—in this example into two aliquots into the two exit ports 960.

As disclosed above, the sample module 100 is configured for enabling delivery of the prepared sample 400 to the reaction module 200. In some examples, delivery of the prepared sample 400 to the reaction module 200 is via gravity, or by applying a centrifugal force to the device 10, for example manually (for example by shaking the device 109 vigorously) or by inserting the device 10 into a suitable centrifuge. Additionally, or alternatively, the device 10 can further comprise a mechanical pump member 700, configured for selectively providing a pulse of pressurized air into the sample chamber 120. For example, such a pump member 700 can comprise a flexible bag 710, for example made from rubber or silicone, that in an unstressed condition has an internal volume V occupied by air or other gas. The bag 710 is fixed to the cap 190 (or alternatively to any suitable part of the sample module casing 110) and has an exit 720 coupled to a corresponding opening 195 provided in the cap or sample module casing. When the bag 710 is squeezed, for example between the fingers of a user, the air or gas previously accommodated in the volume V is rapidly evacuated into the sample chamber 120 effectively providing a pressure pulse for driving the prepared sample 400 that is in the sample chamber 120 into the reaction module 200.

In any case, the transfer of the prepared sample 400 from the sample chamber 120 into the reaction module 200 can be facilitated by providing a vacuum or near vacuum in each of the reaction chambers 250, and/or by providing vents 290 for venting gases to the external environment E from reaction chambers 250. Such vents 290 can be provided in the reaction module casing for example and can include for example suitable filters 295 for allowing only such venting of gases, while preventing ingress of contaminants into the reaction chambers 250 from the external environment E via the vents 290. In some embodiments, suitable filters that may be useful may include Nitrocellulose or any equivalent filter.

The device 10 is configured for further accommodating a fourth quantity of at least one chaotropic agent (not shown), which can be provided in at least one of the sample module 100 and the reaction module 200, prior to use of the device with a user. In other words, one or both of the sample module 100 and the reaction module 200 comes with the fourth quantity of at least one chaotropic agent already accommodated in the device 10, ready for use by the user.

In at least one application of device 10, the corresponding sample chamber 120 has an internal volume of 10 ul-20 ul, while each reaction chamber 250 has a respective internal volume of about 20 ul each, furthermore, the aforesaid first quantity of at least one protease is about 2 ul, the aforesaid second quantity of the solubilizing liquid is about 20 ul, the respective third quantity of the respective reaction mixture in each reaction chamber 250 is about 20 ul, and the aforesaid fourth quantity of the at least one chaotropic agent is about 2 ul, so as the total volume of the reaction including all reagents should be about 20 ul.

As will become clearer herein, the device 10 is further configured for enabling reacting of each respective aliquot of the prepared sample 400 with each respective reaction mixture 510 in the respective reaction chamber 250 to produce at least one amplification product, particularly when the device 10 is used in the system 20.

Furthermore, the device 10 is further configured for enabling detecting a respective test parameter associated with the production of the at least one amplification product.

In at least this example, each aforesaid respective test parameter is in the form of a visually detectable specific color associated with respective reaction mixture subsequent to interaction of said respective aliquot of the prepared sample 400 with the respective reaction mixture 510 in the respective reaction chamber 250.

In this connection, at least a part of the reaction module 200, in particular at least a part of the reaction module casing 210, is transparent to allow each such specific color to be externally observed, recorded and/or quantified. It should be noted that in some embodiments, color signal may be either visually detected or alternatively, using any suitable means, for example, ODmeter and the like.

Additionally, or alternatively, each such respective test parameter is detectable as any one of a specific fluorescence parameter, a specific pH value, or electric charge, conductivity, ATP production or pyrophosphate precipitates each being associated with respective reaction mixture 510 subsequent to interaction of said respective aliquot of the prepared sample 400 with the respective reaction mixture 510 in the respective reaction chamber 250. For example, the reaction module 200 is configured for enabling, for each reaction chamber 250, the respective specific fluorescence parameter, the respective said specific pH value, the respective said electric charge caused by release of hydrogen and production of a hydrogen potential, the respective ATP production (the pyrophosphate (PPi) formed in the DNA polymerase reaction is converted to ATP by ATP sulfurylase and the ATP production is continuously monitored by the firefly luciferase) or the respective pyrophosphate precipitates (pyrophosphate with Calcium, might be measurable in a spectrophotometer) to be externally detected, recorded and/or quantified.

Thus, in use of the above example of the device 10 for the detection of at least one nucleic acid sequence of interest, for example, any nucleic acid sequence of at least one pathogen in at least one sample, each reaction chamber 250 comprises therein the respective third quantity of the respective reaction mixture 510, and the sample module 100 comprises therein the first quantity of at least one protease, and the device is configured for enabling selective delivery of the second quantity of a solubilizing liquid into the sample module 100.

In at least an alternative variation of the above example, the solubilizing liquid can be provided in the sample module 100, prior to use of the device with a user. In other words, the sample module 100 comes with second quantity of a solubilizing liquid 320 already accommodated in the sample chamber 120 ready for use by the user. Thus, in use of such an example of the device 10 for the detection of at least one nucleic acid sequence of interest, for example, any nucleic acid sequence of at least one pathogen in at least one sample, each reaction chamber 250 comprises therein the respective third quantity of the respective reaction mixture 510, and the sample module 100 comprises therein the first quantity of at least one protease, and the second quantity of the solubilizing liquid, wherein the sample module 100 is configured for initially maintaining the first quantity of at least one nucleic acid sequence of interest, for example, any nucleic acid sequence of at least one protease separate from the second quantity of the solubilizing liquid, and for selectively enabling mixing of the first quantity of at least one protease with the second quantity of a solubilizing liquid.

For example, in such an example the sample chamber 120 can be configured for separately accommodating therein the plurality of sample preparation agents 300 and for enabling selectively mixing of the plurality of sample preparation agents 300 to provide the prepared sample 400. For example, the sample module 100 can instead comprise a first sub-chamber configured for accommodating the first quantity of at least one protease therein, prior to use of the device with a user, and a second sub-chamber, different from the first sub-chamber, wherein the second sub-chamber is configured for accommodating the second quantity of a solubilizing liquid therein, prior to use of the device with a user, and wherein the sample module comprises a third sub-chamber configured for accommodating the sample when the device is used with respect to a user. In such a case, the sample module can be configured for selectively enabling fluid communication between the first sub-chamber, the second sub-chamber, and the third sub-chamber to enable mixing of the plurality of sample preparation agents 300 to provide the prepared sample 400. For example, the sample module can comprise a membrane separating the second sub-chamber from the first sub-chamber and the third sub-chamber, and wherein the membrane is selectively rupturable to enable mixing of the plurality of sample preparation agents 300 and sample 390 to provide the prepared sample 400. Alternatively, the sample module 100 can comprise a barrier member separating the second sub-chamber from the first sub-chamber and the third sub-chamber, and the barrier member comprises a valve member that is selectively openable to enable mixing of the plurality of sample preparation agents 300 and the sample 390 to provide the prepared sample 400.

In any case, in use of the above examples of the device 10 for the detection of at least one nucleic acid sequence of interest, for example, any nucleic acid sequence of at least one pathogen in at least one sample, the respective device can also further comprise the fourth quantity of the at least one chaotropic agent.

Furthermore, in use of the above examples of the device 10 for the detection of at least one nucleic acid sequence of interest, for example, any nucleic acid sequence of at least one pathogen in at least one sample, the at least one sample preparation agent/s, that may comprise in some embodiments, at least one protease of said first quantity is proteinase K (PK), and/or the solubilizing liquid of said second quantity comprises water, and/or the at least one chaotropic agent of said fourth quantity is guanidine hydrochloride, and/or the amplification reaction is a loop mediated isothermal amplification reaction (LAMP), and/or at least one of said third quantity of said at least one first reaction mixture comprises at least one set of primers specific for at least one nucleic acid sequence of said at least one nucleic acid sequence of interest, for example, any nucleic acid sequence of at least one pathogen; for example, at least one of said third quantity of said at least one second reaction mixture comprises at least one set of primers specific for at least one control nucleic acid sequence.

In any case, in use of the above examples of the device 10 for the detection of at least one nucleic acid sequence of interest, for example, any nucleic acid sequence of at least one pathogen in at least one sample, the sample is at least one of a biological sample and an environmental sample, and/or, the pathogen is a viral pathogen; for example, the viral pathogen is at least one corona virus (CoV), and for example the CoV is Severe acute respiratory syndrome (SARS) CoV-2.

Referring to FIG. 5, the system 20 for detecting at least one nucleic acid sequence of interest, for example, any nucleic acid sequence of at least one pathogen in at least one sample, according to one example of the presently disclosed subject matter, comprises at least one device 10, for example as disclosed herein with reference to FIG. 4, or alternative variations thereof as herein disclosed, and a heating apparatus 30 configured for heating at least the respective reaction module 200 to a predetermined range of temperatures above ambient. Such a heating apparatus 30 is configured for being coupled to the device 10 and comprises a heating system configured for directing heat to the device 10 when coupled thereto. For example, the heating system of the heating apparatus 30 can comprises electrical heating coils, or a microwave heating system, or indeed a water bath that is pre-heated to the required temperature.

According to an aspect of the presently disclosed subject matter, the predetermined range of temperatures includes a temperature of 65° C.+/−5° C., and/or a temperature of 95° C.+/−5° C.

According to an aspect of the presently disclosed subject matter, the test parameter is in the form of a visually detectable specific color associated with respective reaction mixture subsequent to interaction of the respective aliquot of the prepared sample 400 with the respective reaction mixture 510 in the respective reaction chamber 250. In such a case, at least a part of the reaction module 200, in particular at least a part of the reaction module casing 210, is transparent to allow each such specific color to be externally observed by a user, for example a lab technician. However, it is also possible for each such specific color to be externally recorded and/or quantified using suitable equipment, for example imaging equipment.

In any case, the system 20 can further comprise detection apparatus 50 configured for detecting the test parameters associated with each of the respective said reaction chambers. For example, where test parameter for each respective reaction chamber 250 is a respective specific color, the detection apparatus 50 can be configured for determining a respective wavelength of the respective said specific color.

In another example, where the test parameter for each respective reaction chamber 250 is a respective said specific fluorescence parameter, the detection apparatus can be configured for determining a respective fluorescence value of the respective said specific fluorescence parameter. In another example, where the test parameter for each respective reaction chamber 250 is a respective said specific pH parameter, the detection apparatus can be configured for determining a respective pH value of the respective said specific pH parameter.

The system 20 can be used, for example as follows, in a method for the detection and monitoring of at last one nucleic acid sequence of interest, for example, any nucleic acid sequence of at least one pathogen in at least one sample. In some embodiments, the method comprising the steps of: In a first step (a), the sample or at least one aliquot thereof is contacted with an effective amount of at least one protease to obtain at least one prepared sample. The second step (b), involves subjecting the at least one prepared sample of (a), or at least one aliquot thereof to at least one amplification reaction under isothermal conditions suitable for the production of at least one amplification product detectable by a test parameter, that may be reflected by any detectable signal. It should be understood that the amplification reaction/s are performed using at least one set of primers specific for at least one nucleic acid sequence of the nucleic acid sequence of interest, for example, any nucleic acid sequence of at least one pathogen. In some embodiments, the at least one of steps (a) and (b) may further comprise contacting the sample with at least one chaotropic agent [for example, guanidume hydrochlorid]. The detection of a detectable signal indicates the presence of the pathogen in the examined sample.

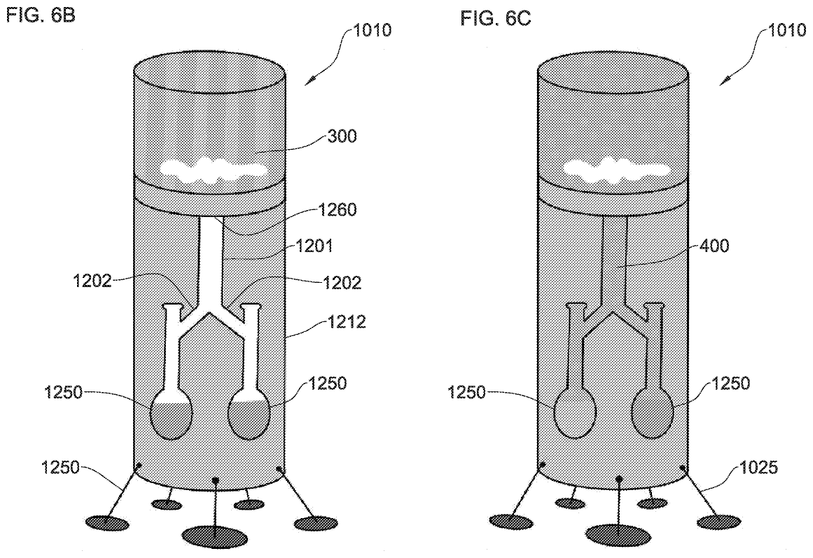

Referring to FIGS. 6A, 6B and 6C, a device for enabling detection of at least one nucleic acid sequence of interest, for example, any nucleic acid sequence of at least one pathogen in at least one sample according to a second example of the presently disclosed subject matter, generally designated 1010, is similar to the first example of the device 10 or alternative variations thereof as disclosed herein, mutatis mutandis, and comprises an sample module 1100 and a reaction module 1200, similar to the sample module 100 and a reaction module 200 of the first example or alternative variations thereof.

As will become clearer below, according to an aspect of the presently disclosed subject matter the device 1010 is also part of a system for detecting at least one nucleic acid sequence of interest, for example, any nucleic acid sequence of at least one pathogen in at least one sample, for example of system 20 as disclosed herein mutatis mutandis.

In at least this example, the sample module 1100 and the reaction module 1200 are integrated into a unitary article thereby facilitating operation thereof, in particular for detecting the at least one nucleic acid sequence of interest, for example, any nucleic acid sequence of at least one pathogen in the at least one sample when used as part of the system 20 (FIG. 5).

As with the first example and alternative variations thereof, mutatis mutandis, the sample module 1100 is also configured for accommodating therein a plurality of sample preparation agents 300 including at least a sample to be tested 390, a first quantity of at least one protease 310, and a second quantity of a solubilizing liquid 320, and for enabling mixing of the plurality of sample preparation agents 300 to provide a prepared sample 400. In this example, the sample to be tested 390 is provided to the sample module 1100 via a cartridge or cassette 1370, the first quantity of at least one protease 310 is provided in a first chamber 1115, and a second quantity of a solubilizing liquid 320 is provided in a second chamber 1125 within the sample module 1100. For example, the cartridge or cassette 1370 is transversely insertable into the sample module 1100 via a lateral opening 1375, and when inserted is in longitudinally stacked relationship with respect to the first chamber 1115 and the second chamber 1125 along the longitudinal axis LA1 of the device 1010.

For example, a membrane can be provided between the first quantity of at least one protease 310 and the second quantity of a solubilizing liquid 320, and another membrane between the first quantity of at least one protease 310 (or the second quantity of a solubilizing liquid 320 depending on which is closer to the cartridge or cassette 1370) and the cartridge or cassette 1370, and the sample module 1100 configured such that when the cartridge or cassette 1370 is inserted into the sample module 1100 the membranes can rupture thereby enabling the sample preparation agents 300 to mix together to form the prepared sample 400 (FIG. 6B).

The sample module 1100 has an outlet port 1150 in fluid communication with a reaction module inlet 1260 of the reaction module 1200.

The reaction module 1200 in the second example includes two reaction chambers 1250 (though in alternative variations of this example the reaction module can have a plurality of reaction chambers including more than two reaction chambers 1250), in the form of respective vessels that are enclosed in the reaction module casing 1212. The reaction chambers 1250, in operation of the device 1010, each includes a respective third quantity of a respective reaction mixture 510 adapted for amplification reaction under isothermal conditions, in a similar manner to the first example and alternative variations thereof, mutatis mutandis. In this example, a conduit 1201 is provided from the reaction module inlet 1260, provided at the upper end of the reaction module 1200, and the conduit 1201 bifurcates into two conduit branches 1202, each of which is coupled to an upper end of a respective reaction chamber 1250.

Thus, it is anticipated that any quantity of prepared sample 400 flowing into the reaction module 1200 is automatically split into two aliquots, one aliquot flowing into each reaction chamber 1250. In at least this example at least the lower end of the reaction module 1200, in particular at least the lower end of the reaction module casing 1212 is transparent for enabling at least visually detection (and optionally recording and/or quantifying) of the respective test parameters associated with operating the device 1010, in a similar manner to the first example and alternative variations thereof, mutatis mutandis. Thus, in this example, at least part of each of the reaction chambers 1250 are visible through the reaction module casing 1212, and each respective test parameter is in the form of a visually detectable specific color associated with respective reaction mixture subsequent to interaction of said respective aliquot of the prepared sample 400 with the respective reaction mixture 510 in the respective reaction chamber 1250. However, additionally or alternatively, each such respective test parameter is detectable as any one of a specific fluorescence parameter, a specific pH value, conductivity, electric charge, (caused by release of hydrogen and production of a hydrogen potential), the respective ATP production (the pyrophosphate (PPi) formed in the DNA polymerase reaction is converted to ATP by ATP sulfurylase and the ATP production is continuously monitored by the firefly luciferase) or the respective pyrophosphate precipitates (pyrophosphate with Calcium, might be measurable in a spectrophotometer), each being associated with respective reaction mixture 510 subsequent to interaction of said respective aliquot of the prepared sample 400 with the respective reaction mixture 510 in the respective reaction chamber 1250. For example, the reaction module 1200 is configured for enabling, for each reaction chamber 1250, the respective specific fluorescence parameter, the respective said specific pH value, or the respective said electric charge, said conductivity, or said the respective ATP production (the PPi formed in the DNA polymerase reaction is converted to ATP by ATP sulfurylase and the ATP production is continuously monitored by any detectable moiety, for example, firefly luciferase) or pyrophosphate precipitates (e.g., pyrophosphate with Calcium, might be measurable in a spectrophotometer) to be externally detected, recorded and/or quantified. The device 1010 is configured for further accommodating a fourth quantity of at least one chaotropic agent (not shown), which can be provided in at least one of the sample module 1100 and the reaction module 1200, prior to use of the device with a user. In other words, one or both of the sample module 1100 and the reaction module 1200 comes with the fourth quantity of at least one chaotropic agent already accommodated in the device 1010, ready for use by the user.

Optionally, the device 1010 comprises a plurality of legs 1025.

Thus, in operation of the device 1010, a sample 390 is deposited onto the cartridge or cassette 1370, and the cartridge or cassette 1370 is inserted into the reaction module 1200 (FIG. 6A). Thereafter the sample preparation agents 300 are mixed together in the sample module 1200 to form the prepared sample 400 (FIG. 6B), which is allowed to pass into the reaction module 1200 (FIG. 6C) via the outlet port 1150 and the reaction module inlet 1260. The prepared sample 400 then splits into two aliquots, each flowing into a different one of the two reaction chambers 1250, and the device 1010 can then be heated via the system 20 to allow the reactions to proceed in each reaction chamber 1250, and the results of the reactions can be determined, via the respective test parameters.

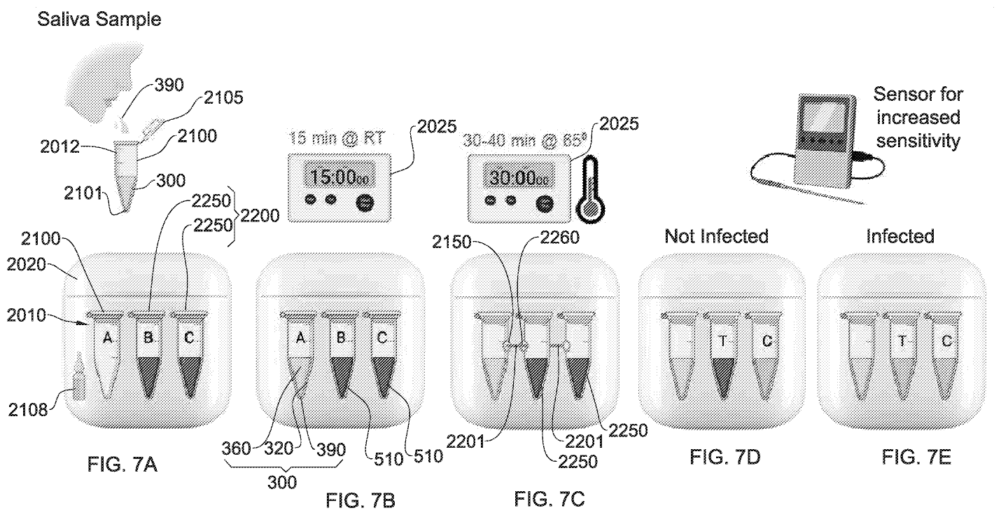

Referring to FIGS. 7A, 7B, 7C, 7D and 7E, a device for enabling detection of at least one nucleic acid sequence of interest, for example, any nucleic acid sequence of at least one pathogen in at least one sample according to a third example of the presently disclosed subject matter, generally designated 2010, is similar to the first example of the device 10 or the second example of the device 1010, or alternative variations thereof, as disclosed herein, mutatis mutandis, and comprises an sample module 2100 and a reaction module 2200, similar to the sample module 100 and a reaction module 200 of the first example or alternative variations thereof mutatis mutandis, or similar to the sample module 1100 and a reaction module 1200 of the second example or alternative variations thereof mutatis mutandis. According to an aspect of the presently disclosed subject matter the device 2010 is also part of a system 2020 for detecting at least one nucleic acid sequence of interest, specifically, a pathogen in at least one sample, for example similar to system 20 as disclosed herein mutatis mutandis.

In at least this example, the sample module 2100 and the reaction module 2200 can be integrated into a unitary article, or can be provided as two separate units that are connected together during operation thereof, and in any cases are used as part of the system 2020.

As with the first example and the second example, and alternative variations thereof, mutatis mutandis, the sample module 2100 is also configured for accommodating therein a plurality of sample preparation agents 300 including at least a sample to be tested 390, a first quantity of at least one protease 310, and a second quantity of a solubilizing liquid 320, and for enabling mixing of the plurality of sample preparation agents 300 to provide a prepared sample 400. In this example, the sample module 2100 is in the form of a tube, closed at a closed end 2101 and open at the an open end 2102, and having a cap 2105 for selectively opening or closing the open end 2012. The sample to be tested 390 is provided to the sample module 1100 via the open end 2012, together with the first quantity of at least one protease 310, and a second quantity of a solubilizing liquid 320, which for example can be provided under pressure via ampule 2108.

Once all the sample preparation agents 300 are in the sample module 2100, they can be mixed together, for example using a vibrator (not shown) which can be part of the system 2020.

The sample module 2100 has an outlet port 2150 in fluid communication with a reaction module inlet 2260 of the reaction module 2200. Such a connection can be integral or alternatively, suitable connections, for example Luer locks, can be provided to connect the sample module with the reaction module using suitable tubing.

The reaction module 2200 in the second example includes two reaction chambers 2250 (though in alternative variations of this example the reaction module can have a plurality of reaction chambers including more than two reaction chambers 2250), in the form of respective vessels that, in operation of the device 2010, each includes a respective third quantity of a respective reaction mixture 510 adapted for amplification reaction under isothermal conditions, in a similar manner to the first example, second example, and alternative variations thereof, mutatis mutandis. In this example, a conduit 2201 is provided from the reaction module inlet 2260, and passes through the two reaction chambers 2250 at a mid-point thereof, and has openings within each of the reaction chambers 2250.

Thus, it is anticipated that any quantity of prepared sample 400 flowing into the reaction module 2200 first fills up the first reaction chamber 2250 providing thereto one aliquot (up to the level of the conduit), and then continues to flow to the next reaction chamber 2250 via the next part of the conduit to provide the aliquot of prepared sample 400 flowing into the second reaction chamber 2250.

The device 2010 is configured for further accommodating a fourth quantity of at least one chaotropic agent (not shown), which can be provided in at least one of the sample module 2100 and the reaction module 2200, prior to use of the device with a user. In other words, one or both of the sample module 2100 and the reaction module 2200 comes with the fourth quantity of at least one chaotropic agent already accommodated in the device 2010, ready for use by the user.

In at least this example at least the lower end of each reaction chamber 2250 is transparent for enabling at least visually detection (and optionally recording and/or quantifying) of the respective test parameters associated with operating the device 2010, in a similar manner to the first example and alternative variations thereof, mutatis mutandis. Thus, in this example, each respective test parameter is in the form of a visually detectable specific color associated with respective reaction mixture subsequent to interaction of said respective aliquot of the prepared sample 400 with the respective reaction mixture 510 in the respective reaction chamber 2250. However, additionally or alternatively, each such respective test parameter is detectable as any one of a specific fluorescence parameter, a specific pH value, conductivity or electric charge caused by release of hydrogen, each being associated with respective reaction mixture 510 subsequent to interaction of said respective aliquot of the prepared sample 400 with the respective reaction mixture 510 in the respective reaction chamber 2250. For example, the reaction module 2200 is configured for enabling, for each reaction chamber 2250, the respective specific fluorescence parameter, the respective said specific pH value, or the respective said electric charge or said conductivity, or the respective ATP production or the respective pyrophosphate precipitates to be externally detected, recorded and/or quantified.

The system 2020 includes a heating arrangement 2025 for selectively enabling heating of the device 2010 as in the first example, mutatis mutandis.

Thus, in operation of the device 2010, a sample 390 is deposited into the reaction module 2200 (FIG. 7A). In at least one application of this example, the at least one protease 310 includes lyophilized Proteinase K, while the solubilizing liquid 320 comprises water, or any other buffer. In some specific embodiments were water are used as a solubilizing liquid, an RNAse, DNAse free water may be used.

Thereafter the sample preparation agents 300 are mixed together in the sample module 2200 to form the prepared sample 400 (FIG. 7B), for example by vibrating the sample module 2100. The sample is incubated for about 10 to 20 minutes, specifically, about 10 or less, or, 11, 12, 13, 14, 15, 16, 17, 18, 19, 20 or more minutes, specifically, about 15 minutes at a temperature ranging between about 18 to 24+/−5° C. degrees Celsius, specifically, 18, 19, 20, 21, 22, 23, 24 degrees Celsius+/−5° C., more specifically, about 20 to 22 degrees Celsius, or more specifically, at room temperature. Subsequent to incubation with the protease, the prepared sample is transferred to the reaction chamber. However, in some alternative and non-limiting embodiments, the method of the invention may further comprise an additional step of inactivation of the protease. Such step may be performed for example by heat inactivation. More specifically, by heating the sample up to about 95+/−5° C. degrees Celsius for a sufficient time period, specifically for about 2 to 10 minutes, more specifically, about 2, 3, 4, 5, 6, 7, 8, 9, or 10 minutes, most specifically, for about 5 minutes. The prepared and inactivated sample is then transferred to the reaction mixture. Then, after about 15 minutes at room temperature (with or without the optional inactivation step), at least part of the prepared sample 400 is allowed to pass into the two reaction chambers 2250 of the reaction module 2200 (FIG. 7C) via the outlet port 2150 and the tubing, such that one aliquots flows into each one of the two reaction chambers 2250. The reaction chambers 2250, which also have the respective reaction mixture 510 therein, can also be vibrated to mix the contents of each reaction chamber 2250. The device 2010 is heated to about 65+/−5° C. degrees Celsius by the heating arrangement for between 30 and 40 minutes, for example. In at least one application of this example, the right reaction chamber 2250 marked “C” operates as a “control”, while the left reaction chamber 2250 marked “T” operates as a “test”.

The results of the reactions can be determined, via the respective test parameters—for example if the contents of left reaction chamber 2250 marked “T” turn yellow, it is indicative of a positive test result, specifically, the presence of the examined nucleic acid sequence of interest, for example, any nucleic acid sequence of at least one pathogen in the sample, while if the contents of left reaction chamber 2250 marked “T” are pink, it is indicative of a negative test result. To provide an indication of reliability of the test results, the contents of right reaction chamber 2250 marked “C” should turn yellow in either case, thereby providing a positive control. FIG. 7D shows a negative, i.e. “not infected” test parameter result, while FIG. 7E shows an example of a positive, i.e. “infected” test parameter result.

Sensor 2300 can optionally be provided to detect pH, or any other detectable signal provided by at least one molecule intercalating to the reaction product. Non-limiting examples for such product may include compounds providing a fluorescent or radioactive signal. Examples for applicable compounds that provide a fluorescent signal may include cyber green, or any thymidine analogue, and can be configured for detecting incorporation of fluorescence to the DNA amplifies in the PCR reaction.

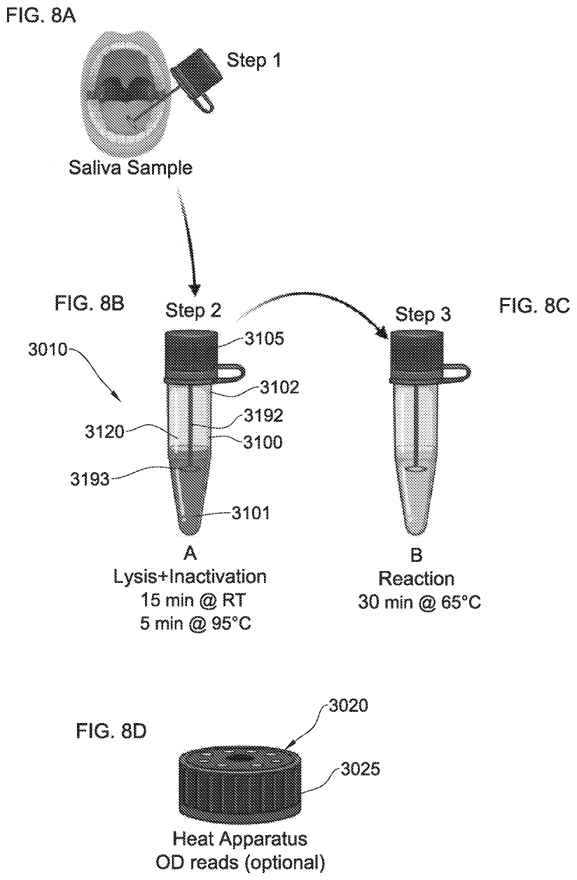

Referring to FIGS. 8A, 8B. 8C and 8D, a device for enabling detection of at least one nucleic acid sequence of interest, for example, any nucleic acid sequence of at least one pathogen in at least one sample according to a fourth example of the presently disclosed subject matter, generally designated 3010, is similar to the first example of the device 10 or the second example of the device 1010 or the third example of the device 2010, or alternative variations thereof, as disclosed herein, mutatis mutandis, but with some differences, as will become clearer. The device 3010 comprises an integrated sample/reaction module 3100, having functions similar in some ways to both the sample module 100 and a reaction module 200 of the first example or alternative variations thereof mutatis mutandis, or similar to the sample module 1100 and the reaction module 1200 of the second example, or similar to the sample module 2100 and the reaction module 2200 of the third example, or alternative variations thereof mutatis mutandis. According to an aspect of the presently disclosed subject matter the device 3010 is also part of a system 3020 for detecting at least one nucleic acid sequence of interest, for example, any nucleic acid sequence of at least one pathogen in at least one sample, for example similar to system 20 as disclosed herein mutatis mutandis.

Thus, in this example, the single integrated sample/reaction module 3100 has a single chamber which serves as both the sample module and the reaction module.

In a similar manner as with the first example and the second example, and alternative variations thereof, mutatis mutandis, the integrated sample/reaction module 3100 is also configured for accommodating therein a plurality of sample preparation agents 300 including at least a sample to be tested 390, a first quantity of at least one protease 310, and a second quantity of a solubilizing liquid 320, and for enabling mixing of the plurality of sample preparation agents 300 to provide a prepared sample 400. In this example, the integrated sample/reaction module 3100 is in the form of a tube having an internal chamber 3120, closed at a closed end 3101 and open at the an open end 3102, and having a cap 3105 for selectively opening or closing the open end 3012.

The cap 3105 includes a sampling member 3192 in the form of a rod projecting from an inner part of the cap 3105 such that when the cap closes the open end 3102 the sampling member 3192 is inside the chamber 3120. For example, the sampling member 3192 can include a loop 3193 at a free end of the rod of the sampling member 3192 for facilitating obtaining the sample 390 from any one of a nose, mouth and throat of a subject.

The sample to be tested 390 is provided to the integrated sample/reaction module 3100 via the open end 3012, together with the first quantity of at least one protease 310, and a second quantity of a solubilizing liquid 320, which for example via an ampule.

Once all the sample preparation agents 300 are in the integrated sample/reaction module 3100, they can be mixed together, either manually or for example using a vibrator (e.g., vortex, not shown) which can be part of the system 3020.

The chamber 3120 also acts as a reaction chamber, and thus a third quantity can be inserted into the chamber 3120 of a respective reaction mixture 510 adapted for amplification reaction under isothermal conditions, in a similar manner to the first example, second example, and alternative variations thereof, mutatis mutandis, via the open end 3102.

The device 3010 is configured for further accommodating a fourth quantity of at least one chaotropic agent (not shown), which can be provided in the integrated sample/reaction module 3100, prior to use of the device with a user or can be inserted therein during use.

In at least this example at least the lower end of each integrated sample/reaction module 3100 is transparent for enabling at least visually detection (and optionally recording and/or quantifying) of the respective test parameters associated with operating the device 2010, in a similar manner to the first example and alternative variations thereof, mutatis mutandis. Thus, in this example, each respective test parameter is in the form of a visually detectable specific color associated with respective reaction mixture subsequent to interaction of the prepared sample 400 with the respective reaction mixture 510 in the chamber 3120. However, additionally or alternatively, each such respective test parameter is detectable as any one of a specific fluorescence parameter, a specific pH value, a specific conductivity, an electric charge caused by release of hydrogen and production of a hydrogen potential, the respective ATP production reflecting PPi production by the polymerase or the respective pyrophosphate precipitates each being associated with respective reaction mixture 510 subsequent to interaction of said respective aliquot of the prepared sample 400 with the respective reaction mixture 510 in the chamber 3120. For example, the integrated sample/reaction module 3100 is configured for enabling the respective specific fluorescence parameter, the respective said specific pH value, or the respective said conductivity or said electric charge, said ATP production reflecting PPi production by the polymerase or the pyrophosphate precipitates to be externally detected, recorded and/or quantified.

The system 3020 includes a heating arrangement 3025 for selectively enabling heating of the device 3010 as in the first example, mutatis mutandis.

Thus, in operation of the device 3010, a sample 390 is obtained from the user (FIG. 8A) and deposited into the chamber 3120 (FIG. 8B).

Thereafter the sample preparation agents 300 are mixed together in the chamber 3120 to form the prepared sample 400 and thereafter the reaction mixture 510 is provided to the chamber 3120 (FIG. 8B), for example by vibrating the integrated sample/reaction module 3100. Then, after about 15 minutes at room temperature the integrated sample/reaction module 3100 is heated to about 95 degrees Celsius using the heating system 3025 (FIG. 8D). The device 3010 is then heated to about 65+/−5° C. degrees Celsius by the heating arrangement 3025 for between 30 and 40 minutes, for example.

The results of the reactions can be determined, via the respective test parameters—in cases that a pH sensitive visible dye is used, for example if the contents of the chamber 3120 turn yellow (FIG. 8C), it is indicative of a positive test result, while if the contents of chamber 3120 marked “T” are pink, it is indicative of a negative test result.

In the method claims that follow, alphanumeric characters and Roman numerals used to designate claim steps are provided for convenience only and do not imply any particular order of performing the steps.

Finally, it should be noted that the word “comprising” as used throughout the appended claims is to be interpreted to mean “including but not limited to”.

While there has been shown and disclosed examples in accordance with the presently disclosed subject matter, it will be appreciated that many changes may be made therein without departing from the scope of the presently disclosed subject matter as set out in the claims.

A further aspect of the invention relates to a method for the detection and monitoring of at last one nucleic acid sequence of interest in at least one sample. In some embodiments, the nucleic acid sequence of interest, may be any nucleic acid sequence of at least one pathogen. More specifically, in some embodiments the methods of the invention may comprise the following steps:

First step (a), involves contacting the sample or at least one aliquot thereof with an effective amount of at least one sample preparation agent/s, to obtain at least one prepared sample.

The next step (b) involves subjecting the at least one prepared sample of (a), or at least one aliquot thereof to at least one amplification reaction under isothermal conditions suitable for the production of at least one amplification product detectable by a detectable signal, that is further referred to herein as a test parameter. It should be noted that at least one of such amplification reaction/s may be performed using at least one set of primers specific for at least one nucleic acid sequence of the nucleic acid sequence of interest.

In some embodiments, the sample preparation agent/s may comprise at least one nucleic acid stripping compound. In some embodiments, such compound may be at least one protease. It should be noted that in some further embodiments, the method of the invention may further comprise in at least one of steps (a) and (b), contacting the sample with at least one chaotropic agent. More specifically, the at least one cheotropic agent may be contacted with the sample in step (a), during the preparation and incubation with the at least one protease. Alternatively, the at least one cheotropic agent may be contacted with the examined sample during the amplification reaction. In yet some further embodiments, the at least one cheotropic agent may be added to both steps, during the preparation of the sample in step (a) and during the amplification reaction in step (b).

It should be noted that a detection of a detectable signal indicates the presence of the nucleic acid sequence of interest, for example, any nucleic acid sequence of at least one pathogen in the examined sample.

In some embodiments, the sample preparation agent/s may comprise at least one protease.

In more specific embodiments, the sample preparation step (a), may involve incubation of the sample with the at least one protease or with any solutions or buffers comprising the protease, for a sufficient period of time under suitable conditions. Alternatively, the sample preparation step (a), may involve incubation of the sample with any sample preparation agent/s or with any solutions or buffers comprising the sample preparation agents, for a sufficient period of time, under suitable conditions.

More specifically, in some embodiments the sample is incubated for about 10 to 20 minutes, specifically, about 10 or less, or, 11, 12, 13, 14, 15, 16, 17, 18, 19, 20 or more minutes, specifically, about 15 minutes at a temperature ranging between about 18 to 24 degrees Celsius, specifically, 18, 19, 20, 21, 22, 23, 24 degrees Celsius, more specifically, about 20 to 22 degrees Celsius, or more specifically, at room temperature. It should be noted that at this sample preparation step, the protease containing solution or buffer may further contain at least one further agent. Non-limiting examples for relevant additional agents may include any surfactant or detergent. For example, at least one polysorbate-type nonionic surfactant such as Polysorbate 20, also known as tween 20, and the like, at any suitable concentration. It should be understood that the invention encompasses the addition of any additional reagent or buffer required for the preparation step (a). As noted above, in some embodiments, the preparation step may further comprise incubation with at least one cheotropic agent. Thus, the protease solutions or buffers may comprise in some embodiments, at least one cheotropic agent.

Still further, subsequent to incubation with the protease, the prepared sample is transferred to the reaction chamber. However, in some alternative and non-limiting embodiments, the method of the invention may further comprise an additional step of heat inactivation of the sample or of any sample preparation agent, that may comprise in some embodiments, any nucleic acid stripping agent. Such step may be performed for example by heat inactivation. In some embodiments, the heat inactivation step may be performed by heating the sample to between about 70° C. to 100° C., specifically, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100° C. In some embodiments this step involves heating the sample to about 95° C.,+/−5° C. More specifically, by heating the sample up to about 95 degrees Celsius,+/−5° C., for a sufficient time period, specifically for about 2 to 20 minutes, more specifically, about 2, 3, 4, 5, 6, 7, 8, 9, 10, 11, 12, 13, 14, 15, 16, 17, 18, 19, 20 minutes or more, most specifically, for about 5 minutes. In yet some further embodiments, the sample is heated for about 10 minutes. The prepared and inactivated sample is then transferred to the reaction mixture. In some embodiments, the sample preparation agent/s may comprise at least one nucleic acid stripping compound. In some embodiments, such compound may be at least one protease. Accordingly, in some further embodiments, the het inactivation step indicated herein may inactivates the protease. To detect nucleic acid sequence specific for a particular pathogen of interest in the sample, the next step (b) of the methods of the invention involves amplification reaction under isothermal conditions.

As used herein, the term “amplification” refers to increasing the number of copies of a nucleic acid molecule used as a template, such as a gene, a fragment of a gene, or any control element or fragment thereof, for example, of any nucleic acid sequence of interest, for example, any nucleic acid sequence of at least one pathogen or of a positive or negative control. As will be discussed in more detail herein after, in some specific and non-limiting embodiments, such nucleic acid molecule may be any nucleic acid sequence of any gene of interest, for example, any gene of at least one pathogen, for example, of a viral pathogen. In more specific embodiments, at least a portion of SARS-COV2 nucleic acid molecule. In some particular and non-limiting embodiments of the invention, Gene N of SARS-CoV2 may be used as a template. The products of an amplification reaction are called amplification products. An example of in vitro amplification is the polymerase chain reaction (PCR), in which a sample (such as a biological sample from a subject, or any environmental sample) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Examples of in vitro amplification techniques include real-time PCR, quantitative real-time PCR (qPCR), reverse transcription PCR (RT-PCR), quantitative RT-PCR (qRT-PCR); loop-mediated isothermal amplification; reverse-transcription LAMP (RT-LAMP); strand displacement amplification; transcription-mediated amplification, transcription-free isothermal amplification; repair chain reaction amplification; ligase chain reaction amplification; gap filling ligase chain reaction amplification; coupled ligase detection and PCR; and NASBA™ RNA transcription-free amplification.

As indicated above, the method of the invention comprises the step of performing an amplification reaction using at least one set of primers specific for the any nucleic acid sequence of interest, for example, any nucleic acid sequence of at least one pathogen to be detected. However, in some embodiments, the method of the invention may further comprise controls that ensure that the reaction steps are appropriately performed. In some embodiments, the control may include performance of at least one additional control amplification reaction, performed in parallel or subsequently, with at least one additional aliquot of the sample. Thus, in some embodiments, the methods, kits, devices and systems of the invention encompass the use of control reaction mixtures, and even of control samples. The term “Control” refers to a reference standard, for example a positive control or negative control. A positive control is known to provide a positive test result. A negative control is known to provide a negative test result. However, the reference standard can be a theoretical or computed result, for example a result obtained in a population of reactions. Thus, a positive control may involve the use of primers specific for nucleic acid sequences known or expected to be present in the examined sample. For example, in case of a sample of a mammalian subject, specifically, a human subject, a positive control reaction mixture may comprise the use of primers specific for a human gene. In case of a sample obtained from a human source, any human gene may be used as a template for a suitable positive control. Non limiting example for nucleic acid sequence used as a positive control may be primers specific for the human POP7 gene that encodes the human ribonuclease P protein subunit p20 protein. Thus, according to such embodiments, a control amplification reaction may be performed using at least one set of primers specific for the POP7 gene. This amplification reaction is expected to produce a POP7 amplification product in case all steps of sample preparation and amplification reaction were performed appropriately. In such case, a negative result in the test amplification reaction performed using at least one set of primers specific the at least one pathogen, and a positive result (appearance of an amplification product) in the positive control reaction, indicates and ensures, at least partially, that nucleic acid sequences of the examined nucleic acid sequence of interest, for example, any nucleic acid sequence of at least one pathogen are indeed not present in the sample. Positive results (appearance of at least one amplification product) in both, the test and the positive control samples, may indicate that the nucleic acid sequence of the pathogen are present in the sample, thereby indicating the presence of the nucleic acid sequence of interest, for example, any nucleic acid sequence of at least one pathogen in the tested sample. It should be noted that the method of the invention may further use at least one negative control reaction that may be directed in some embodiments to nucleic acid sequences that are not expected to be present in the sample. A negative control may be provided by a reaction mixture comprising primers for nucleic acid sequences that are irrelevant for the sample. Such negative control reactions ensures the specificity of the reagents, and the specificity of a positive result (appearance of an amplification product specific for the nucleic acid sequence of interest, for example, the pathogen). Thus, a positive result of the test sample, a positive result of the positive control sample, and a negative result (no amplification product appears) in the negative control sample indicates the existence of the pathogen in the examined sample.

Therefore, in some embodiments, step (b) of the method of the invention may further comprise subjecting at least one aliquot of the prepared sample of (a), to at least one amplification reaction using at least one set of primers specific for at least one control nucleic acid sequence. As indicated above, such control may be a positive control, or alternatively, or additionally, a negative control. It should be however appreciated that the use of control samples, or at least relative scale obtained from control samples may be also encompassed by the methods of the invention. As a control sample, the method of the invention may use a nucleic acid sequence of the searched nucleic acid sequence of interest, for example, any nucleic acid sequence of at least one pathogen or at least one sample known to comprise the pathogen. Alternatively, a control sample may include sample known to be negative for the specific searched pathogen or any nucleic acid sequence thereof. A further negative control may include in some embodiments, a control reaction in which no template has been added.

Typical amplification reactions contain primers (e.g., one, two, four or six primers), sample (which may or may not contain template to which the primers bind), nucleotides (corresponding to G, A, T and C), a buffering agent (1 mM to 5 mM Tris or 1.5 mM to 5 mM Tris or an equivalent buffer thereof), one or more salts (e.g., (NH₄)₂S0₄, NaCl, MgSO₄, MgCl₂, etc), a bacterial or archaebacterial polymerase (which may or may not be thermostable and may or may not have strand displacing activity), and any necessary cofactors and optional detergents, etc. Examples of thermostable polymerases that can be used for amplification, and specifically for in isothermal amplification reactions include, but are not limited to Taq, Tfi, Tzi, Tth, Pwo, Pfu, Q5®, Phusion®, OneTaq®, Vent®, DeepVent®, Klenow(exo-), Bst 2.0 and Bst 3.0 (New England Biolabs, Ipswich. MA). PyroPhage® (Lucigen. Middleton, Wi), Tin DNA polymerase, GspSSD LF DNA polymerase, Rsp (OptiGene. Horsham, UK) and phi29 polymerase, etc.

As noted above, the amplification reaction requires the use of primers, for example primers specific for nucleic acid sequences of the pathogen or for at least one control nucleic acid sequence. Primers, as used herein, are short nucleic acids, generally DNA oligonucleotides 10 nucleotides or more in length (such as 10-60, 15-50, 20-40, 20-50, 25-50, or 30-60 nucleotides in length). Primers may be annealed to a complementary target DNA strand by nucleic acid hybridization to form a hybrid between the primer and the target DNA strand, and then extended along the target DNA strand by a DNA polymerase enzyme. Primer pairs or sets of primers (such as 2, 3, 4, 5, 6, or more primers) can be used for amplification of a target nucleic acid, e.g., by PCR, LAMP, RT-LAMP, or other nucleic acid amplification methods known in the art. In some embodiments, where the pathogen to be detected is at least one viral pathogen, primers specific for a viral nucleic acid sequence are used. In some particular and non-limiting embodiments, where the pathogen to be detected is the SARS-CoV2 virus, primers specific for nucleic acid sequences of SARS-CoV2 virus, are used by the methods of the invention.

In some specific embodiments, where the Gene N of SARS-CoV2 is used as a template, suitable primers that may be used by the invention may comprise the nucleic acid sequence as denoted by any one of SEQ ID NO. 7, 8, 9, 10, 11 and 12.

As noted above, in some embodiments, the sample preparation agent/s may comprise at least one nucleic acid stripping compound. In some embodiments, such compound may be at least one protease. Still further, as indicated above, the first step of the method of the invention is the sample preparation step that may comprise contacting the sample with an effective amount of at least one protease, or any solution or mixture comprising the protease. In some embodiments such protease may be any serine protease.

As shown by the invention, at least one protease is used for sample preparation. A protease (also called a peptidase or proteinase) is an enzyme that catalyzes (increases the rate of) proteolysis, the breakdown of proteins into smaller polypeptides or single amino acids, by cleaving the peptide bonds within proteins by hydrolysis.

Thus, in some embodiments, proteases useful in the present invention may be at least one serine protease. Serine proteases (or serine endopeptidases) are enzymes that cleave peptide bonds in proteins, in which serine serves as the nucleophilic amino acid at the (enzyme's) active site. Serine proteases fall into two broad categories based on their structure: chymotrypsin-like (trypsin-like) or subtilisin-like. In some particular and non-limiting embodiments, the protease used by the devices, systems, kits and methods of the invention for sample preparation may be proteinase K, or any variants, conjugates or derivatives thereof, or any solution, reagent, buffer or mixture thereof. Proteinase K (EC 3.4.21.64, protease K, endopeptidase K, Tritirachium alkaline proteinase, Tritirachium album serine proteinase, Tritirachium album proteinase K) is a broad-spectrum serine protease. The enzyme was discovered in 1974 in extracts of the fungus Engyodontium album (formerly Tritirachium album). Proteinase K is able to digest hair (keratin), hence, the name “Proteinase K”. The predominant site of cleavage is the peptide bond adjacent to the carboxyl group of aliphatic and aromatic amino acids with blocked alpha amino groups. It is commonly used for its broad specificity. This enzyme belongs to Peptidase family S8 (subtilisin). The molecular weight of Proteinase K is 28,900 daltons (28.9 kDa).

Activated by calcium, the enzyme digests proteins preferentially after hydrophobic amino acids (aliphatic, aromatic and other hydrophobic amino acids). Although calcium ions do not affect the enzyme activity, they do contribute to its stability. Proteins will be completely digested if the incubation time is long and the protease concentration high enough. Upon removal of the calcium ions, the stability of the enzyme is reduced, but the proteolytic activity remains. Proteinase K has two binding sites for Ca2+, which are located close to the active center, but are not directly involved in the catalytic mechanism. The residual activity is sufficient to digest proteins, which usually contaminate nucleic acid preparations. Therefore, the digestion with Proteinase K for the purification of nucleic acids is usually performed in the presence of EDTA (inhibition of metal-ion dependent enzymes such as nucleases).

Proteinase K is also stable over a wide pH range (4-12), with a pH optimum of pH 8.0. An elevation of the reaction temperature from 37° C. to 50-60° C. may increase the activity several times, like the addition of 0.5-1% sodium dodecyl sulfate (SDS) or Guanidinium chloride (3 M), Guanidinium thiocyanate (1 M) and urea (4 M). The above-mentioned conditions enhance proteinase K activity by making its substrate cleavage sites more accessible. Temperatures above 65° C.+/−5° C., trichloroacetic acid (TCA) or the serine protease-inhibitors AEBSF, PMSF or DFP inhibit the activity. Proteinase K will not be inhibited by Guanidinium chloride, Guanidinium thiocyanate, urea, Sarkosyl, Triton X-100, Tween 20, SDS, citrate, iodoacetic acid, EDTA or by other serine protease inhibitors like Na-Tosyl-Lys Chloromethyl Ketone (TLCK) and Na-Tosyl-Phe Chloromethyl Ketone (TPCK).

In some embodiments, either the sample preparation step (a), or the amplification reaction of step (b), or even both steps, may comprise adding to the sample at least one cheotropic agent. It means that either the sample preparation step, and/or the amplification reaction step may be performed in the presence of at least one cheotropic agent.

A chaotropic agent is a molecule in water solution that can disrupt the hydrogen bonding network between water molecules (i.e. exerts chaotropic activity). This has an effect on the stability of the native state of other molecules in the solution, mainly macromolecules (proteins, nucleic acids) by weakening the hydrophobic effect. For example, a chaotropic agent reduces the amount of order in the structure of a protein formed by water molecules, both in the bulk and the hydration shells around hydrophobic amino acids, and may cause its denaturation.

Common chaotropic agents used include guanidinium chloride, n-butanol, ethanol, lithium perchlorate, lithium acetate, magnesium chloride, phenol, 2-propanol, sodium dodecyl sulfate, thiourea, and urea. In some embodiments, the at least one chaotropic agent may be guanidine hydrochloride. Guanidinium chloride or guanidine hydrochloride, usually abbreviated GuHCl and sometimes GdnHCl or GdmCl, is the hydrochloride salt of guanidine.

Guanidinium chloride is a strong chaotrope and one of the strongest denaturants used in physiochemical studies of protein folding. It also has the ability to decrease enzyme activity and increase the solubility of hydrophobic molecules. At high concentrations of guanidinium chloride (e.g., 6 M), proteins lose their ordered structure, and they tend to become randomly coiled, i.e. they do not contain any residual structure.

As noted above, the method of the present disclosure comprises the step of amplification reaction to amplify either the nucleic acid sequence of interest (e.g., of a pathogen) that may be present in the sample, or the control nucleic acid sequence (either positive control that is present in the sample, or a negative control that is not present in the sample). According to some embodiments, such steps are performed using isothermal conditions.

Isothermal amplification methods provide detection of a nucleic acid target sequence in a streamlined, exponential manner, and are not limited by the constraint of thermal cycling. Although these methods can vary considerably, they all share some features in common. For example, because the DNA strands are not heat denatured, all isothermal methods rely on an alternative approach to enable primer binding and initiation of the amplification reaction: a polymerase with strand-displacement activity. Once the reaction is initiated, the polymerase must also separate the strand that is still annealed to the sequence of interest. Isothermal amplification reactions are performed using constant thermal conditions, specifically, either moderate temperature reactions (ranges between about 25 to about 40° C.) or higher temperature (ranges between about 50 to about 65° C.).

Still further, isothermal methods typically employ unique DNA polymerases for separating duplex DNA. DNA polymerases with this ability include Klenow exo-, Bsu large fragment, and phi29 for moderate temperature reactions (25-40° C.) and the large fragment of Bst DNA polymerase for higher temperature (50-65° C.) reactions

In some embodiments, the amplification reaction performed under isothermal conditions may be at least one of Loop-Mediated Isothermal Amplification (LAMP) that may be performed at 65° C., Strand Displacement Amplification (SDA), that may be performed at 60° C., Helicase-Dependent Amplification (HDA), that may be performed at 65° C., Recombinase Polymerase Amplification (RPA), that may be performed at 37° C., and Nucleic Acid Sequences Based Amplification (NASBA), that may be performed at 40-55° C.

More specifically, LAMP uses 4-6 primers recognizing 6-8 distinct regions of target DNA. A strand-displacing DNA polymerase initiates synthesis and 2 of the primers form loop structures to facilitate subsequent rounds of amplification. LAMP is rapid, sensitive, and amplification is so extensive that the magnesium pyrophosphate produced during the reaction can be seen by eye, making LAMP well-suited for field diagnostics.

SDA, or a similar approach, Nicking Enzyme Amplification Reaction (NEAR), relies on a strand-displacing DNA polymerase, typically Bst DNA Polymerase, Large Fragment or Klenow Fragment (3′-5′ exo-), to initiate at nicks created by a strand-limited restriction endonuclease or nicking enzyme at a site contained in a primer. The nicking site is regenerated with each polymerase displacement step, resulting in exponential amplification. NEAR is extremely rapid and sensitive, enabling detection of small target amounts in minutes. SDA and NEAR are typically utilized in clinical and biosafety applications.

HDA employs the double-stranded DNA unwinding activity of a helicase to separate strands, enabling primer annealing and extension by a strand-displacing DNA polymerase. Like PCR, this system requires only two primers. HDA has been employed in several diagnostic devices and FDA-approved tests.

RPA uses a recombinase enzyme to help primers invade double-stranded DNA. T4 UvsX, UvsY, and a single stranded binding protein T4 gp32 form D-loop recombination structures that initiate amplification by a strand-displacing DNA polymerase. RPA is typically performed at −37° C. and, unlike other methods, can produce discrete amplicons up to 1 kb.

Still further, NASBA and Transcription Mediated Amplification (TMA) are both isothermal amplification methods that proceed through RNA. Primers are designed to target a region of interest; one of the primers must include the promoter sequence for T7 RNA polymerase at the 5′ end. NASBA and TMA reactions are utilized in a range of clinical diagnostics.

In some embodiments, such amplification step is a loop mediated isothermal amplification reaction (LAMP).

More specifically, Loop-mediated isothermal amplification (LAMP) refers to a method for amplifying nucleic acid. The method is a single-step amplification reaction utilizing a DNA polymerase with strand displacement activity (e.g., Notomi et al., Nucl. Acids. Res. 28:E63, 2000), utilizing 4-6 primers designed to amplify the gene target through creation of stem-loop structures that aid in synthesizing new DNA by the polymerase. Synthesis of DNA occurs rapidly at a constant temperature, unlike PCR which requires specialized equipment for temperature cycling. LAMP is also highly specific, since several primers are used to amplify a specific nucleic acid sequence. More specifically, at least four primers, which are specific for eight regions within a target nucleic acid sequence, are typically used for LAMP; however, in some embodiments, two primers may be used for LAMP. The primers may include in some embodiments a forward outer primer (F3), a backward outer primer (B3), a forward inner primer (FIP), and a backward inner primer (BIP). A forward loop primer (Loop F or LF), and/or a backward loop primer (Loop B or LB) can also be included in some embodiments. The amplification reaction produces a stem-loop DNA with inverted repeats of the target nucleic acid sequence. To amplify RNA sequences using LAMP, reverse transcriptase (RT) is added to the reaction. This variation is referred to as RT-LAMP. In contrast to PCR, LAMP and RT-LAMP are carried out at a constant temperature and do not require a thermal cycler. Thus, in some embodiments, either one set of primers or a multiplex of several sets of primers may be used. More specifically, in some embodiments, 1, 2, 3, 4, 5, 6, 7, 8, 9, 10, 11, 12, 13, 14, 1, 16, 17, 18, 19, 20, or more sets of primers may be used.

It should be noted that the production of an amplification product indicates the existence of the specific nucleic acid sequence (either of the nucleic acid sequence of interest, e.g., nucleic acid sequence of at least one pathogen, or the control) may be detected by any parameter, for example, change in pH, change in conductivity, change in electric charge, change in ATP production reflecting PPi production by the polymerase, or change in pyrophosphate precipitates. In some embodiments, the change in pH may be detected using any pH sensitive indicator.

Thus, in general, in one aspect, an aqueous amplification reaction mix is provided that includes: a polymerase, 1.5 mM to 5 mM Tris or an equivalent thereof, a single pH sensitive dye, dNTP, one or more primers; and one or more templates wherein the reaction mix changes color only during amplification reaction. In embodiments of the method, change in color of pH sensitive dyes resulting from amplification is observed in any buffer at a concentration equivalent to 1.5 mM to 5 mM Tris such as 2 mM, 2.5 mM, 3 mM, 3.5 mM, 4 mM, 4.5 mM or 5 mM Tris or any other concentration in the specified range. A person of ordinary skill in the art will be acquainted with Tris buffer and equivalent buffers that are standard within the art such as Tricine, Bicine, TAPS, MOBS, and Hepes (Sigma Aldrich, St. Louis, MO) including DIPSO, TAPSO, HEPPSO, POPSO, TEA, EPPS, HEPBS, AMPD and TABS (Sigma Aldrich, St. Louis, MO).

In some embodiments the LAMP reaction is performed using a reaction mixture comprising a pH sensitive indicator dye providing a detectable signal upon production of at least one amplification product.

Thus, in some embodiments, a pH sensitive indicator dye is used by the invention to provide a detectable signal upon production of at least one amplification product. A pH-sensitive indicator dye is a dye that undergoes a color change as pH increases or decreases. In the context of the present disclosure, pH-sensitive indicator dyes can be used to detect nucleic acid amplification products. When a DNA polymerase incorporates a deoxynucleoside triphosphate into a nascent DNA, the released by-products include a pyrophosphate moiety and a hydrogen ion. Thus, as nucleic acid amplification products increase in a reaction, the pH of the solution decreases, resulting in a color change of the pH-sensitive dye. In some embodiments, the pH sensitive indicator dye is a colored dye detectable in visible light. In some embodiments, the change in color of the amplification reaction may be visible by eye. As such, the detecting step may be done by a naked eye.

The detection may be qualitative or quantitative. In some embodiments, for example, the method may comprise simply detecting whether there has been a change in the color during the amplification reaction, thereby indicating the presence or absence of an amplification product. In some embodiments, the color of the reaction may be compared to a color chart or the color of one or more controls, e.g., an amplification reaction that does not contain any template and/or an amplification reaction that contains a different template that is amplified in the amplification reaction, thereby allowing a user to determine if the reaction contains a product or not. In other embodiments, for example, the method may comprise quantifying the change in the color of the amplification reaction, thereby indicating the amount of product in the amplification product. In some embodiments, the color of the reaction may be compared to a color chart or the color of one or more controls, e.g., amplification reactions that contain varying amounts of a different template that is amplified in the amplification reaction, thereby allowing a user to quantify the amount of product and/or template in the amplification reaction. In some cases, the color of the reaction may be read within 1 hour, e.g., 5 minutes-50 minutes, after the reaction starts.

The pH-sensitive dye used may be selected according to any characteristic. e.g., their color change (i.e., whether they change from violet to yellow, red to yellow, or yellow to red, etc.), the initial pH of the amplification reaction (e.g., whether the reaction initially has a pH of greater than pH 8.0, a pH of 7.5-8.5, or a pH of 6.5-7.5, etc., and whether the color change is going to be detected using a machine (e.g., a colorimeter, fluorimeter or spectrophotometer) by human eye (i.e., without the aid of a colorimeter, fluorimeter or spectrophotometer). The selected dye generally changes color in a pH range at which the polymerase is operational (e.g., a pH of 5-10).

There are a wide range of pH-sensitive dyes that can be used in the present method. Examples of pH sensitive dyes that change color at different pH values are described below. These examples are not intended to be limiting. Suitable visible dyes include: neutral red, which has a clear-yellow color when the pH is higher than 8 and a red color when the pH is less than 6.8; phenol red, which has a red color when the pH is higher than 8 and a yellow color when the pH is less than 6.4; cresol red, which has a reddish-purple color when the pH is higher than 8.8 and a yellow color when the pH is less than 7.2; thymol blue, which has a blue color when the pH is higher than 9.6 and a yellow color when the pH is less than 8.0; phenolphthalein, which has a fuchsia color when the pH is higher than 10 and colorless when the pH is less than 8.3; and naphtholphthalein, which has a greenish color when the pH is higher than 8.7 and a pale-reddish color when the pH is less than 7.3. The term “visual” includes those dyes that can be detected by the “naked eye” where the “naked eye” refers to visualization without instrumentation. The “naked eye” includes the use of contact lenses and/or spectacles. The contact lenses/spectacles may include lenses and/or tinting to enhance or eliminate certain wavelengths of light.

Other examples of pH indicators include, methyl yellow, methyl orange, bromophenol blue, naphthyl red, bromocresol green, methyl red, azolitmin, nile blue, thymolphthalein, alizarin yellow, salicyl yellow, nitramine, phenol red, cresol red, neutral red, m-cresol purple, bromocresol purple, naphtholphthalein, thymol blue and naphtolphthalein. The color may transition outside the range of traditional DNA polymerase tolerances, but the principle of amplification detection may be applied to alternate detection methods with an indicator appropriate for desired pH range. pH-sensitive fluorescent dyes can be detected using a fluorometer. Like visual dyes mentioned above, pH-sensitive fluorescence dyes have different levels of fluorescence emission or a shift of peak emission wavelength at different pH. Both the change in brightness and the shift in peak absorption can be easily detected using systems that are equipped with proper filter sets.

It should be understood that the result of the reaction can be either assessed by eye or by a colorimetric measure such as an ODmeter, and the like.

In yet some alternative embodiments, the pH sensitive indicator dye is a fluorescent indicator dye. Fluorescent indicator dye refers to a fluorescent compound that responds to changes in environmental conditions (such as pH or metal ion concentration) by changes in fluorescence properties. In some examples, fluorescence of a fluorescent indicator dye is increased by a change in pH. The fluorescent indicator dye can be detected by any suitable method, including visually (e.g., under ambient or ultraviolet light) or using instrumentation for detection of fluorescence (such as a fluorimeter or real-time PCR system). Exemplary fluorescent indicator dyes include, for example, calcein, hydroxynaphthol blue, Mag-Fura-2 and Magnesium Green (Life Technologies, Grand Island, NY) and Fluo-2 Mg, Fura-2 Mg, Indo-1 Mg, and Asante Magnesium Green (TEF Labs, Austin, TX). Other fluorescent pH indicators include 2′,7′-bis-(2-carboxyethyl)-5-(and-6)-carboxyfluorescein, acetoxymethyl ester (BCECF-AM) (Life Technologies, Grand Island, NY) which at pH 9 has an absorbance/emission profile of Aₘₐx 500 nm/Emₘax535 nm, 5-(and-6) carboxy SNARF-1 which features a shift in fluorescence based on pH. At high pH (pH 9) SNARF-1 maximum absorbance/emission at Aₘₐx 575 nm/Emₘax 650 nm.

In some examples, fluorescence from the fluorescent indicator dyes useful in the methods disclosed herein is visibly detectable (for example, by eye, such as a colorimetric reagent), while in other examples, the fluorescence is detectable using an instrument, such as a fluorimeter or real-time PCR platform.

Visual and fluorescent dyes including those mentioned above can be chemically modified to have altered colorimetric properties in response to pH changes. These modifications can create dyes that are either brighter or change color at a narrower pH range and thus allow a better detection.

In some embodiments, where a pH sensitive dye is not comprised within the reaction mixture, the change of pH as a function of the production of the amplification product, may be measured either manually or mechanically (pH meter etc.).

In yet some alternative embodiments, the LAMP reaction is performed using a reaction mixture comprising at least one detectable compound capable of intercalating into the a double strand DNA, thereby providing a detectable signal upon production of at least one amplification product. In some embodiments, the detectable compound may be detected either directly or indirectly. In yet some further embodiments, the detectable compound may be a fluorescent compound. In yet some further embodiments, such detectable compound may be SYBER green. More specifically, SYBR Green I (SG) is an asymmetrical cyanine dye used as a nucleic acid stain that binds to DNA. The resulting DNA-dye-complex absorbs best 497 nanometer blue light (λₘₐₓ=497 nm) and emits green light (λₘₐₓ=520 nm). The stain preferentially binds to double-stranded DNA, but will stain single-stranded (ss) DNA with lower performance. SYBR Green can also stain RNA with a lower performance than ssDNA.

In yet some alternative embodiments, for detecting and/or quantitating the amplification product, EdU (5-Ethynyl-2′-deoxyuridine) that is capable of intercalating to the nucleic acid sequence, is used by the methods of the invention. More specifically, EdU is a thymidine analog that can be incorporated into replicating DNA. The alkyne handler can be used for subsequent ligation to azide-containing molecules through a highly efficient click chemistry reaction.

In yet some further alternative embodiments. the presence of an amplification product may be detected by measuring the change in other parameters, for example, electric charge or conductivity of the sample after amplification reaction. In some embodiments, change in electric charge measured or detected by the methods of the invention may be caused by release of hydrogen and production of a hydrogen potential. More specifically, the change in electric charge is detected and/or measured by the methods of the invention, using for example a membrane that enables accumulation of protons. Electricity would be measured in the chamber in which the accumulation of the protons is enabled, by any suitable means.

In yet some further embodiments, the detection of DNA polymerase activity that reflects the production of the amplification product in the reaction chamber may be assessed in some embodiments by measuring the production of inorganic pyrophosphate (PPi). In some particular and non-limiting embodiments, such PPi may be assessed by measuring the production of ATP. More specifically, in some embodiments, by an enzymatic luminometric inorganic pyrophosphate (PPi) detection assay (ELIDA). The PPi formed in the DNA polymerase reaction is converted to ATP by ATP sulfurylase and the ATP production is continuously monitored by the firefly luciferase.

In yet some further embodiments, the production of the amplification product may be performed in some embodiments by measuring and/or detecting inorganic pyrophosphate (PPi) that reflects DNA polymerase activity. Precipitates of the pyrophosphate with Calcium, may be measured, by any suitable means, for example, using in a spectrophotometer.

As indicated above, the detection means for detecting an amplification product produced in the sample tested by the methods, kits, systems and devices of the invention can be a fluorophore, a radiolabel, or any other signal-emitting, signal inducing, or otherwise chemically or physically discernible. The various detection systems are well known to those skilled in the art.

The term “detectable” as used herein refers to the presence of a detectable signal generated from a detectable chemical reaction that is immediately detectable by observation, instrumentation, or film. The term “detectable signal” (also referred to by some aspects of the invention as a test parameter) as used herein refers to any compound or means that leads to occurrence of, or a change in, a signal that is directly or indirectly detectable (observable) either by visual observation or by instrumentation, thereby reflecting the production of amplification product in accordance with the invention. Typically, the detectable signal is detectable in an optical property (“optically detectable”) as reflected by a change in the wavelength distribution patterns, or intensity of absorbance, or a combination of such parameters.

The method of the invention enables detection of at least one nucleic acid sequence of interest, for example, any nucleic acid sequence of at least one pathogen, and specifically the presence of at least one nucleic acid sequence of at least one pathogen in any sample. Thus, in some embodiments, the devices, systems, kits and methods of the invention may be suitable for detecting at least one pathogen, and specifically, nucleic acid molecules thereof, in any surface, substance or any sample thereof. The terms “sample”, “test sample” and “specimen” are used interchangeably in the present specification and claims and are used in its broadest sense. They are meant to include both biological and environmental samples and may include an exemplar of synthetic origin. This term refers to any media that may contain the nucleic acid sequence of interest, for example, any nucleic acid sequence of at least one pathogen and may include fluid, cell and/or tissue samples. In some embodiments herein, the biological sample is a fluid sample. Fluid sample include, but are not limited to, saliva, mucosa, feces, serum, urine, blood, plasma, cerebral spinal fluid (CSF), milk, bronchoalveolar lavage (BAL) fluid, rinse fluid obtained from wash of body cavities, phlegm, pus. Still further, biological samples including samples taken from various body regions (nose, throat, vagina, ear, eye, skin, sores), food products (both solids and fluids) and swabs taken from medicinal instruments, apparatus, materials), samples from various surfaces [hospitals, elderly homes, food manufacturing facilities, slaughter houses pharmaceutical equipment (catheters etc), food preparation or packaging products), solutions and buffers], sewage etc. In some embodiments, the sample is at least one of a biological sample and an environmental sample.

Biological samples may be provided from animal, including human, fluid, solid (e.g., stool) or tissue, as well as liquid and solid food and feed products, food designed for human consumption, a sample including food designed for animal consumption, food matrices and ingredients such as dairy items, vegetables, meat and meat by-products, waste and sewage. In some embodiments, biological samples may include saliva, mucosa (nasal or oral swab samples), feces, serum, blood, urine, anterior nares (nasal swab) specimen collected by a healthcare professional or by onsite or home self-collection (using a flocked or spun polyester swab Nasopharyngeal (NP) specimens throat swab. Biological samples and specimens may be obtained from human as well as from all of the various families of domestic animals, as well as feral or wild animals, including, but not limited to, such animals as ungulates, bear, birds, fish, lagamorphs, rodents, etc.

As indicated herein before, the present invention provides methods, kits, devices and systems that may be applicable for detecting at least one nucleic acid sequence of interest, for example, any nucleic acid sequence of at least one pathogen in any sample, including environmental samples. More specifically, environmental samples include environmental material such as surface matter, earth, soil, water, air and industrial samples, as well as samples obtained from food and dairy processing instruments, apparatus, equipment, utensils, disposable and non-disposable items.

These examples are not to be construed as limiting the sample types applicable to the present invention. The sample may be any media, specifically, a liquid media that may contain the nucleic acid sequence of interest, for example, any nucleic acid sequence of at least one pathogen. Typically, substances, surfaces and samples or specimens that are a priori not liquid may be contacted with a liquid media which is used and tested by the methods, kits, devices and systems of the invention of the invention.

In some embodiments, the methods, kits, devices and systems of the invention may be applicable for detecting at least one nucleic acid sequence of interest, for example, any nucleic acid sequence of at least one pathogen in food or food products and beverages. More specifically, by the term “food”, it is referred to any substance consumed, usually of plant or animal origin. Some non limiting examples of animals used for feeding are cows, pigs, poultry, etc. The term food also comprises products derived from animals, such as, but not limited to, milk and food products derived from milk, eggs, meat, etc. A drink or beverage is a liquid which is specifically prepared for human consumption. Non limiting examples of drinks include, but are not limited to water, milk, alcoholic and non-alcoholic beverages, soft drinks, fruit extracts, etc.

In some embodiments, the present disclosure provides methods, devices, systems and kits for detecting any nucleic acid sequence of interest, for example, any nucleic acid sequence of at least one pathogen. A nucleic acid sequence of interest as used herein refers to any DNA, or any RNA sequence or fragments thereof, or any coding or non-coding or regulatory sequence in chromosomal DNA, or in DNA of any organelle of a eukaryotic cell, for example, mitochondria, chloroplast, amyloplast and chromoplast or any fragment thereof.

Still further, “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties. The nucleic acid molecule according to the invention may be of a variable nucleotide length. For example, in some embodiments, the nucleic acid molecule according to the invention comprises 1-100 nucleotides, e.g., about 10, 20, 30, 40, 50, 60, 70, 80, 90 or 100 nucleotides. In other embodiments the nucleic acid molecule according to the invention comprises 100-1,000 nucleotides, e.g., about 100, 200, 300, 400, 500, 600, 700, 800, 900 or 1000 nucleotides.

As indicated above, in some embodiments, the method of the invention provides the detection of any pathogen in any sample. The present invention provides devices, systems, kits and methods for detecting pathogens in a sample. As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode.

In yet some further embodiments, the methods of the invention may be applicable for detecting a pathogen that may be in further specific embodiment, a viral pathogen or a virus. The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis.

It should be noted that the term “viruses” is used in its broadest sense to include any virus, specifically, any enveloped virus. In some specific embodiments, the viral pathogen may be of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages).

In some embodiments, the viral pathogens of the invention may be DNA viruses, specifically, any virus of the following families: the Adenoviridae family, the Papovaviridae family, the Parvoviridae family, the Herpesviridae family, the Poxviridae family, the Hepadnaviridae family and the Anelloviridae family.

In yet some further specific embodiments, the viral pathogens of the invention may be RNA viruses, specifically, any virus of the following families: the Reoviridae family, Picornaviridae family, Caliciviridae family, Togaviridae family, Arenaviridae family, Flaviviridae family, Orthomyxoviridae family, Paramyxoviridae family, Bunyaviridae family, Rhabdoviridae family, Filoviridae family, Coronaviridae family, Astroviridae family, Bornaviridae family, Arteriviridae family, Hepeviridae family and the Retroviridae family. Of particular interest are viruses of the families Coronaviridae (Middle East Respiratory Syndrome (MERS-CoV), Severe acute respiratory syndrome (SARS-Cov), SARS-CoV2, corona virus), Flaviviridae (Dengue virus, Yellow fever, West nile virus and Zika), adenoviruses, Filoviridae (Ebolavirus), papovaviruses, herpesviruses: simplex, varicella-zoster, Epstein-Barr (EBV), Cytomegalo virus (CMV), pox viruses: smallpox, vaccinia, hepatitis B (HBV), rhinoviruses, hepatitis A (HBA), poliovirus, respiratory syncytial virus (RSV), rubella virus, hepatitis C (HBC), arboviruses, rabies virus, influenza viruses A and B, measles virus, mumps virus, human deficiency virus (HIV), HTLV I and II and Zika virus.

In some specific and embodiments, the devices, systems, kits and methods of the invention may be suitable for detecting at least one corona virus (CoV). CoVs are common in humans and usually cause mild to moderate upper-respiratory tract illnesses. There are four main sub-groupings of coronaviruses, known as alpha, beta, gamma, and delta. The seven coronaviruses known to-date as infecting humans are: alpha coronaviruses 229E and NL63, and beta coronaviruses OC43, HKU1, SARS-CoV and SARS-CoV2, and MERS-CoV (the coronavirus that causes Middle East Respiratory Syndrome, or MERS). The SARS-CoV and SARS-CoV2 are a lineage B beta Coronavirus and the MERS-CoV is a lineage C beta Coronavirus.

Coronaviruses are species in the genera of virus belonging to one of two subfamilies Coronavirinae and Torovirinae in the family Coronaviridae, in the order Nidovirales. Herein this term refers to the entire family of Coronavirinae (in the order Nidovirales). Coronaviruses are enveloped viruses with a positive-sense single-stranded RNA genome and with a nucleocapsid of helical symmetry. The genomic size of coronaviruses ranges from approximately 26 to 32 kilobases, the largest for an RNA virus. The name “coronavirus” is derived from the Latin corona, meaning crown or halo, and refers to the characteristic appearance of virions under electron microscopy (E.M.) with a fringe of large surface projections creating an image reminiscent of a crown. This morphology is created by the viral spike (S) peplomers, which are proteins that populate the surface of the virus and determine host tropism.

There are many CoVs that naturally infect animals, the majority of these usually infect only one animal species or, at most, a small number of closely related species, but not humans. CoV strains that are particular subject of the present invention, due to their extreme virulence and hazard in humans, are those that have been transmitted from animals to humans, specifically the SARS-COV2. Thus, the term Coronaviruses (designated herein CoVs) for the purposes of the present invention encompasses four main sub-groupings of coronaviruses, known as Alpha, Beta, Gamma, and Delta. More specifically, under this term is meant the enveloped viruses with a positive-sense RNA genome (ssRNA+) and with a nucleocapsid of helical symmetry; and also large RNA viruses with the genomic size of ranges from approximately 26 to 32 kilobases; and further viruses with the characteristic morphology of large, bulbous surface projections under electron microscopy, which is created by the viral spike (S) peplomers, i.e. viral surface proteins determining host tropism and immunogenicity.

In some non-limiting embodiments, the present invention may be particularly applicable to the seven CoVs that can infect humans, specifically the Alpha CoVs 229E and NL63, and Beta CoVs OC43, HKU1, SARS-CoV, SARS-Cov2 and MERS-CoV. Specifically, for the Beta-CoVs, which are of the greatest clinical importance concerning humans, these are OC43, and HKU1 of the A lineage, SARS-CoV of the B lineage, and MERS-CoV of the C lineage. MERS-CoV is the first Beta CoV belonging to lineage C that is known to infect humans. The Alpha and Beta CoVs genera descend from the bat gene pool. Coronaviruses infecting animals are also contemplated, in particular the bat coronaviruses HKU4 and HKU5.

Within the above group of human CoVs, of particular relevance to the present invention are the SARS-CoV2 associated with COVID19, SARS CoV associated with Severe Acute Respiratory Syndrome, and the MERS CoV associated with Middle East Respiratory Syndrome, as for being the primary causes of life-threatening infectious diseases and epidemics in humans.

The other human CoVs are believed to cause a significant percentage of all common colds in human adults (primarily in the winter and early spring seasons). In certain individuals CoVs may further be a direct or indirect cause of pneumonia, i.e. direct viral pneumonia or a secondary bacterial pneumonia.

Still further, in some specific embodiments, the method of the invention may be particularly applicable in detecting the Severe acute respiratory syndrome (SARS) CoV-2, and any variants and mutants thereof.

Thus, of particular relevance to the present invention is the SARS CoV2 associated with Severe Acute Respiratory Syndrome 2, as for being the primary cause of life-threatening infectious diseases and epidemics/pandemics in humans referred to as COVID 19 (Coronavirus Disease 2019).

SARS CoV2 is a member of the subgenus Sarbecovirus (beta-CoV lineage B). Its RNA sequence is approximately 30,000 bases in length. SARS-CoV-2 is unique among known betacoronaviruses in its incorporation of a polybasic cleavage site, a characteristic known to increase pathogenicity and transmissibility in other viruses.

With a sufficient number of sequenced genomes, it is possible to reconstruct a phylogenetic tree of the mutation history of a family of viruses. By 11 Feb. 2020, 81 genomes of SARS-CoV-2 had been isolated and reported by the Chinese Center for Disease Control and Prevention (CCDC) and other institutions. A phylogenetic analysis of those samples showed they were “highly related with at most seven mutations relative to a common ancestor”, implying that the first human infection occurred in November or December 2019.

On 11 Feb. 2020, the International Committee on Taxonomy of Viruses (ICTV) announced that according to existing rules that compute hierarchical relationships among coronaviruses on the basis of five conserved sequences of nucleic acids, the differences between what was then called 2019-nCoV and the virus strain from the 2003 SARS outbreak were insufficient to make it a separate viral species. Therefore, they identified 2019-nCoV (now referred to as SARS CoV2) as a strain of severe acute respiratory syndrome-related coronavirus.

Each SARS-CoV-2 virion is approximately 50 to 200 nanometres in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope. In some embodiments, the nucleic acid sequence of the severe acute respiratory syndrome coronavirus 2 isolate (SARS CoV2) Wuhan-Hu-1, complete genome is denoted by NCBI Reference Sequence: NC_045512.2. In yet some further embodiments, the SARS CoV2 nucleic acid sequence is as denoted by SEQ ID NO: 19, and any variants and mutants thereof. In the specific case of the SARS CoV2, a defined receptor-binding domain on S mediates the attachment of the virus to its cellular receptor, angiotensin-converting enzyme 2 (ACE2). It should be however understood that any of the following substitutions revealed for any SARS CoV2 strain, are further encompassed by the present disclosure, more specifically, variants that carry substitutions in the spike protein, for example, the substitutions of the B.1.1.7 strain (also known as the “British variant”), that include N501 Y, P681H, H69-V70 and Y144/145 deletions, A570D, D614G, P681H, T716I, S982A and D1118H. The substitutions of the B.1.1.298 strain that include H69-V70 deletion, Y453F, D614G, 1692V and M12291. The substitutions of the B.1.1.429 strain that include S131, W152C, L452R and D614G; the substitutions of the P2 strain that include E484K, D614G, V1176F, the substitutions of the P1 strain that include L18F, T20N, P26S, D138Y, R190S, K417T, E484K, N501Y, D614G, H655Y, T1027I, V1176F. The substitutions of the B.1.351 strain (also known as 20C/501Y.V2, or the “South African variant”), that has the following variants B.1.351 V1 strain, that include the substitutions of D80A, D215G, L242/A243/L244 deletion, K417N, E484K, N501Y, D614G, A701V; the B.1.351 V2 strain. that include the substitutions of L18F, D80A, D215G, L242/A243/L244 deletion, K417N, E484K, N501Y, D614G, A701V; and the B.1.351 V3 strain, that include the substitutions of D80A, R2461, L242/A243/L244 deletion, K417N, E484K, N501Y, D614G and A701V. It should be noted that in some embodiments, the positions specified herein refer to the spik protein as denoted by SEQ ID NO: 20.

In some specific embodiments, a Coronavirus applicable in the present invention may be Middle East Respiratory Syndrome coronavirus (MERS CoV).

The term MERS CoV as known in the art refers to a lineage C beta coronavirus (+RNA 30 kb) whose primary natural reservoir resides in bats that infect domesticated camels as opportunistic hosts, which go on to infect humans. MERS-CoV genomes are phylogenetically classified into two clades, clade A and B. The earliest cases of MERS were of clade A clusters (EMC/2012 and Jordan-N3/2012), and new cases are genetically distinct (clade B). MERS-CoV is distinct from SARS-CoV and distinct from the common-cold coronavirus and known endemic human betacoronaviruses HCoV-OC43 and HCoV-HKU1. Until 23 May 2013, MERS-CoV had frequently been referred to as a SARS-like virus, or simply the novel coronavirus, and early it was referred to colloquially as the “Saudi SARS”. Over 1,600 cases of MERS have been reported by 2015 and the case fatality rate is >30%. 182 genomes have been sequenced by 2015 (94 from humans and 88 from dromedary camels). All sequences are >99% similar. The genomes can be divided into two clades—A and B—with the majority of cases being cased by clade B. Human and camel strains are intermixed suggesting multiple transmission events.

Like other coronaviruses, the MERS-CoV virion utilizes a large surface spike (S) glycoprotein for interaction with and entry into the target cell. The S glycoprotein consists of a globular S1 domain at the N-terminal region, followed by membrane-proximal S2 domain, a transmembrane domain and an intracellular domain.

Determinants of cellular tropism and interaction with the target cell are within the S 1 domain, while mediators of membrane fusion have been identified within the S2 domain. Through co-purification with the MERS-CoV SI domain, dipeptidyl peptidase 4 (DPP4) was identified as cellular receptor for MERS-CoV. DDP4 is expressed on the surface of several cell types, including those found in human airways, and possesses ectopeptidase activity, although this enzymatic function does not appear to be essential for viral entry.

In yet some other alternative embodiments, the Coronavirus may be Severe Acute Respiratory Syndrome coronavirus (SARS CoV).

Specifically, SARS-CoV has been associated with a viral disorder characterized by high fever, dry cough, shortness of breath (dyspnea) or breathing difficulties, and atypical pneumonia. The complete SARS-CoV has been analyzed and published by Marra et al. 2003. Since then, a large number of SARS strains have been isolated and characterized, and are accessible via the Centers for Disease Control and Prevention (CDC), or the National Center for Biotechnology Information, e.g., the sequence of the SARS-CoV Urbani strain is under Acc. Num. AY278741.

More specially, the SARS coronavirus (SARS-CoV) is a lineage B beta coronavirus that causes severe acute respiratory syndrome (SARS). SARS-CoV is a positive and single stranded RNA virus belonging to a family of enveloped coronaviruses. Its genome is about 29.7 kb. The SARS virus has 13 known genes and 14 known proteins. SARS is similar to other coronaviruses in that its genome expression starts with translation of two large ORFs, 1a and 1b, both of which are polyproteins. The functions of several of these proteins are known, ORFs 1a and 1b encode the replicase and there are four major structural proteins: nucleocapsid, spike, membrane and envelope. It also encodes for eight unique proteins, known as the accessory proteins, all with no known homologues or function.

In yet some further embodiments, the devices, systems, kits and methods of the invention may be applicable for detecting bacterial pathogens in a sample.

The term “bacteria” (in singular a “bacterium”) in this context refers to any type of a single celled microbe. Herein the terms “bacterium” and “microbe” are interchangeable. This term encompasses herein bacteria belonging to general classes according to their basic shapes, namely spherical (cocci), rod (bacilli), spiral (spirilla), comma (vibrios) or corkscrew (spirochaetes), as well as bacteria that exist as single cells, in pairs, chains or clusters.

It should be noted that the term “bacteria” as used herein refers to any of the prokaryotic microorganisms that exist as a single cell or in a cluster or aggregate of single cells. In more specific embodiments, the term “bacteria” specifically refers to Gram positive, Gram negative or Acid fast organisms. The Gram-positive bacteria can be recognized as retaining the crystal violet stain used in the Gram staining method of bacterial differentiation, and therefore appear to be purple-colored under a microscope. The Gram-negative bacteria do not retain the crystal violet, making positive identification possible. In other words, the term ‘bacteria’ applies herein to bacteria with a thicker peptidoglycan layer in the cell wall outside the cell membrane (Gram-positive), and to bacteria with a thin peptidoglycan layer of their cell wall that is sandwiched between an inner cytoplasmic cell membrane and a bacterial outer membrane (Gram-negative). This term further applies to some bacteria, such as Deinococcus, which stain Gram-positive due to the presence of a thick peptidoglycan layer, but also possess an outer cell membrane, and thus suggested as intermediates in the transition between monoderm (Gram-positive) and diderm (Gram-negative) bacteria. Acid fast organisms like Mycobacterium contain large amounts of lipid substances within their cell walls called mycolic acids that resist staining by conventional methods such as a Gram stain.

Of particular interest, a pathogen to be detected by the devices, systems, kits and methods of the invention, may be any bacteria involved in nosocomial infections or any mixture of such bacteria. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality and pose a significant economic burden. In view of the rising levels of antibiotic resistance and the increasing severity of illness of hospital in-patients, this problem needs an urgent solution.

Common nosocomial organisms include Clostridium difficile, methicillin-resistant Staphylococcus aureus, coagulase-negative Staphylococci, vancomycin-resistant Enteroccocci, resistant Enterobacteriaceae, Pseudomonas aeruginosa, Acinetobacter and Stenotrophomonas maltophilia.

The nosocomial-infection pathogens could be subdivided into Gram-positive bacteria (Staphylococcus aureus, Coagulase-negative staphylococci), Gram-positive cocci (Enterococcus faecalis and Enterococcus faecium), Gram-negative rod-shaped organisms (Klebsiella pneumonia, Klebsiella oxytoca, Escherichia coli, Proteus aeruginosa, Serratia spp.), Gram-negative bacilli (Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). Among many others Pseudomonas aeruginosa is an extremely important nosocomial Gram-negative aerobic rod pathogen. In particular and non-limiting embodiments, such microbe of interest may be an antibiotic-resistant bacteria.

Of particular interest are “ESKAPE” pathogens. As indicated herein, these pathogens include but are not limited to Enterococcus faecium, Staphylococcus aurus, Klebsiella pneumoniae, Acinetobacter baumanii, Pseudomonas aeruginosa, and Enterobacter.

Thus, in some embodiments of the invention relate to bacteria of any strain of at least one of E. coli. Pseudomonas aeruginosa, Staphylococcus aureus, Streptococcus pyogenes. Clostidium difficile, Enterococcus faecium, Klebsiella pneumonia, Acinetobacter baumanni and Enterobacter species (specifically, ESKAPE bacteria).

In further embodiments the pathogen according to the present disclosure may be a bacterial cell of at least one of E. coli, Pseudomonas spp, specifically, Pseudomonas aeruginosa, Staphylococcus spp, specifically, Staphylococcus aureus, Streptococcus spp, specifically, Streptococcus pyogenes, Salmonella spp, Shigella spp, Clostidium spp, specifically, Clostidium difficile, Enterococcus spp, specifically, Enterococcus faecium, Klebsiella spp, specifically, Klebsiella pneumonia, Acinetobacter spp, specifically, Acinetobacter baumanni, Yersinia spp, specifically, Yersinia pestis and Enterobacter species or any mutant, variant isolate or any combination thereof.

A lower eukaryotic organism applicable in the present invention as pathogens to be detected by the devices, systems, kits and methods provided by the invention, includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the invention.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

Still further, in certain embodiments the devices, systems, kits and methods of the invention may be suitable for detecting fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous, and possess branched somatic structures (hyphae) surrounded by cell walls containing glucan or chitin or both, and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present invention also provides for devices, systems, kits and methods that may be suitable for detecting a parasitic pathogen. More specifically, “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species.

As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

As shown by the following Examples, the methods provided by the invention are particularly applicable for detecting a viral pathogen, for example the SARS CoV-2 in a patient, thereby demonstrating the feasibility of using the methods of the invention for the diagnosis and monitoring of an infectious disease caused by at least one of the pathogen/s in a subject.

In more specific and non-limiting embodiments, the methods of the invention may be particularly applicable for detecting viral pathogens that may affect airways. In some particular embodiments, the methods of the invention may be applicable for the diagnosis of COVID-19 in a mammalian subject.

Subjects, as used by the invention, encompass any multicellular organism, for example, any living multi-cellular vertebrate organisms, a category that includes both human and non-human animals, such as non-human mammals (such as mice, rats, rabbits, sheep, horses, cows, and non-human primates).

More specifically, SARS-CoV2 has been associated with a viral disorder named COVID-19 which is characterized by numerous symptoms, while the most common symptoms are fever and a dry cough. The third most common symptom is fatigue. Almost 40% of cases suffered from it. ‘Sputum production’ (a thick mucus which is coughed up from the lungs) was experienced by every third person. Sputum is not saliva. It is thick mucus which is coughed up from the lungs.

About 18.6% experienced shortness of breath (‘dyspnoea’). Many of the most common symptoms are shared with those of the common flu or cold while SARS-CoV2 infection rarely causes a runny nose. According to the WHO, people infected with SARS-CoV2 generally develop signs and symptoms, including mild respiratory symptoms and fever, on an average of 5-6 days after infection. While the mean incubation period is 5 to 6 days, the WHO adds that the incubation period can vary in a wide range of between 1 to 14 days.

Symptoms were categorized as mild, severe, or critical and the research article describes these as follows: Critical cases: Critical cases include patients who suffered respiratory failure, septic shock, and/or multiple organ dysfunction or failure, that may lead to death.

Severe cases: This includes patients who suffered from shortness of breath, respiratory frequency ≥30/minute, blood oxygen saturation ≤93%, PaO₂/FiO₂ ratio <300,30 and/or lung infiltrates>50% within 24-48 hours.

Mild cases: The majority (81%) of these coronavirus disease cases were mild cases. Mild cases include all patients without pneumonia or cases of mild pneumonia.

In some embodiments, the methods of the invention may be performed using any means and any suitable devices or systems. In some particular embodiments, the methods of the invention may be performed using any of the devices of the invention as defined herein before, or any of the systems of the invention as defined by other aspects of the invention herein before.

As shown by the Examples, the invention provides pathogen detection assays and kits providing high sensitivity and specificity. The term “sensitivity” is used herein with respect to the ability of the methods of the invention to detect a pathogen, specifically, amplification products that use as a template nucleic acid sequences of a target nucleic acid sequence of interest, for example, any nucleic acid sequence of at least one pathogen, even when said nucleic acid sequence template is present in a low concentration in a sample. In some particular embodiments, the sensitivity of the methods of the invention may detect concentration that may be less than viral load equivalent to Ct qRTPCR cycle of 28.8 and under. Readings collected after 35 and 40 minutes showed the highest True Positive (TP) rate in samples presenting low (Ct<26) and medium (26<Ct<29) Ct values. For Ct values under 28.8, the TP rate reported by RT-LAMP is 93%.

The term “specificity” is used herein with respect to the ability of the methods of the invention to detect a pathogen, specifically, amplification products specific for such pathogen, by preferentially using as a template nucleic acid sequences specific for said target pathogen in a sample. The specific primers used by the reaction mixtures used by the invention bind the intended nucleic acid sequence of the target pathogen for use as a template to produce a specific amplification product, at least 1.5 fold more than any other nucleic acid sequence in said sample, at least 2.0 fold, at least 2.5 fold, at least 5.0 fold, at least 10.0 fold, at least 20.0 fold, at least 50.0 fold, at least 100 fold, at least 200 fold, at least 500 fold, at least 1000 fold, at least 5000 fold, at least 10000 fold, at least 50,000 fold, or at least 100,000 fold. As shown in Example 5 and Table 4, the specificity of the disclosed assay is very high and ranges between about 90-99.999%, specifically, 90, 91, 92, 93, 94, 95, 96, 97, 98, 99, 99.1, 99.2, 99.3, 99.4, 99.5, 99.6, 99.7, 99.8. 99.9% and more. In some specific embodiments the specificity of the assay provided therein is 99.5%.

In yet a further aspect thereof, the present disclosure provides a kit comprising:

First, as a component (a), the kit of the invention comprises an effective amount of at least one sample preparation agent/s. In some embodiments, the sample preparation agent/s may comprise at least one nucleic acid stripping compound. In some embodiments, such compound may be at least one protease. The protease or any other sample preparation agent/s, may be provided in any buffer or solution, either in liquid or in a solid form, for example, as a lyophilized preparation. In some optional embodiments, the at least one protease of the kit of the invention may be provided comprised within a sample preparation module. In more specific embodiments, such sample module may be any of the sample modules disclosed by the invention in connection with any of the devices or any of the systems of the invention.

The kit of the invention may comprise as a component (b), an amplification reaction mixture. In some embodiments, the reaction mixture may comprise any reagent, solution and/or component required for performing and/or improving an amplification reaction. Specifically, any amplification reaction under isothermal conditions. Specific examples for reaction mixtures useful in the kits of the invention are indicated in more detail herein after. In some embodiments, the reaction mixtures provided by the kits of the invention may be comprised within a reaction module. In more specific embodiments, such reaction module may comprise a plurality of reaction chambers, each of such reaction chambers comprises the amplification mixture. As indicated above, the reaction mixture is adapted for amplification reaction under isothermal conditions. In more specific embodiments, the reaction mixture in at least one first reaction chamber may comprise at least one set of primers specific for at least one nucleic acid sequence of at least one nucleic acid sequence of interest, for example, any nucleic acid sequence of at least one pathogen. It should be understood that in some embodiments, the reaction buffer can be provided within at least one reaction chamber. In yet some further embodiments, the at least one reaction chambers may be comprised within the reaction module of the invention.

In yet some further embodiments, the kit of the invention may further comprise at least one chaotropic agent. The chaotropic agent may be provided for use with the at least one protease of the kits of the invention, and therefore, in some embodiments may be provided comprised within at least one of the sample preparation module (a). In yet some alternative embodiments, the at least one cheotropic agents of the kits of the invention may be provided for use with the reaction mixture. In some embodiments, the at least one cheotropic agent may be provided with the reaction module (b). In yet some further embodiments, the at least one cheotropic agent may be provided either within the reaction mixture, or separately, ready for use with the reaction mixture. In some further embodiments, the at least one cheotropic agent may be provided comprised within at least one of the reaction chambers of the reaction module (b).

In yet some further embodiments, the at least one cheotropic agent may be provided adapted for use with both, the at least one protease of (a) and the reaction mixture of (b).

It should be understood that in some embodiments, all the components of the kits of the invention may be provided either separately, or in any mixtures thereof. For example, in cases where the kit comprises the cheotropic agent for use with the at least one protease, both components may be provided either separately, or together as a mixture in liquid or dried (e.g., lyophilized) forms thereof. Another example is the components of the reaction mixture, that may be provided either with or without the at least one cheotropic agents, that may be provided either separately, or as a mixture or any other combination. More specifically, in some embodiments, each and every component of the reaction mixture, with or without the cheotropic agents may be provided separately. In yet some alternative embodiments, all components or any combinations thereof, may be provided together as a mixture in liquid or dried (e.g., lyophilized) forms thereof.

As indicated above, all components of the kits of the invention can be provided separately. In some embodiments, the different components of the kits of the invention may be provided in separate compartments, vesicles or any other separating means. However, it must be understood that in some alternative embodiments, the reaction chambers of the reaction module of the device, systems and kits of the invention, may be in the form of an array. Thus, in some embodiments, the different components of the kits of the invention may be provided separately, in an array. The term “array” as used by the invention refers to an “addressed” spatial arrangement of the plurality of reaction mixtures. Each “address” of the array is a predetermined specific spatial region containing said reaction mixture. For example, an array may be a plurality of vessels (test tubes), plates (or even different predetermined locations in one plate or one slide), micro-wells in a micro-plate each containing different reaction mixtures containing different sets of primers. An array that comprise such plurality of reaction chambers may also be any solid support holding in distinct regions (dots, lines, columns) different reaction mixtures. The array preferably includes built-in appropriate controls, for example, regions without the sample, regions without various reagents of the reaction mixture, for example, polymerase, dNTPs, the specific pH-sensitive dye, the chaotropic agent, or any combinations thereof, or event with buffers alone.

As indicated above, the reaction mixture provided by the kits of the invention is specific for providing an amplification product of a particular nucleic acid sequence of at least one pathogen. However, in some embodiments, control reactions mixtures may be also included by the kits of the invention. More specifically, in some embodiments, at least one of the reaction mixtures provided by the kit of the invention may comprise at least one set of primers specific for at least one control nucleic acid sequence. In yet some further embodiments, reaction mixture specific for at least one control nucleic acid sequence, may be provided comprised within at least one second reaction chamber. Such control reaction mixture may comprise at least one set of primers specific for at least one control nucleic acid sequence.

In some embodiments, the at least one protease provide by the kits of the invention may be at least one serine protease. In yet some further embodiments, the serine protease provided by the kits of the invention may be proteinase K (PK), any variants, conjugates or derivatives thereof, or any solution, reagent, buffer or mixture thereof.

In yet some further embodiments, the at least one chaotropic agent provided by the kits of the invention may be guanidine hydrochloride.

Still further, in some embodiments, the kits of the invention are adapted for amplification reactions under isothermal conditions. In more specific embodiments, such amplification reaction may be any one of LAMP, SDA, HAD, RPA or NASBA. In yet some further embodiments, the amplification reaction is a loop mediated isothermal amplification reaction (LAMP).

In some embodiments, the LAMP reaction mixture further comprises at least one pH sensitive indicator dye. It should be noted that such dye provides a detectable signal upon production of at least one amplification product by the amplification reaction.

In some embodiments, the pH sensitive indicator dye is a colored dye detectable in visible light.

In yet some alternative embodiments, the pH sensitive indicator dye may be a fluorescent indicator dye.

In some alternative embodiments, the kit of the invention provides reaction mixture that supports production of amplification product that comprises a detectable agent. Such detectable compound may be added directly or indirectly to one of the components of the produced amplification product. In some embodiments, such detectable agent may be an agent that is intercalated into the amplification product. In some embodiments, the LAMP reaction mixture further comprises at least one fluorescent compound capable of intercalating into a double strand DNA, thereby providing a detectable signal upon production of at least one amplification product. In some embodiments, such fluorescent compound that intercalates into the nucleic acid amplification product may be SYBER green.

In yet some alternative embodiments, the reaction mixture provided by the kits of the invention may comprise as a compound that intercalates into the amplification product, an analogue of thymidine. In more specific embodiments, such thymidine analog may be 5-Ethynyl-2′-deoxyuridine (EdU). More specifically, EdU is a thymidine analog that can be incorporated into the amplified product. In yet some further embodiments, the EdU may be detected through a click chemistry reaction.

In yet some further alternative embodiments, the kit of the invention may be adapted for detecting the formation of amplification products by measuring pH of the reaction. Thus, in accordance with some embodiments, the kit of the invention may comprise at least one means for detecting or measuring pH, for example, a pH meter or any other suitable pH sensor and recorder, other image analysis or any computerized application thereof.

In yet some further embodiments, the kit of the invention may be adapted for detecting the formation of amplification products by measuring a change in electric charge caused by release of hydrogen and production of a hydrogen potential, or production of pyrophosphate. The kit of the invention may further comprise in accordance with some embodiments, at least one membrane that enables accumulation of protons.

Still further, in some embodiments, the kit of the invention is adapted for detection and monitoring of at last one pathogen in at least one sample.

In yet some further embodiments, such sample may be any sample, for example, at least one of a biological sample or an environmental sample. Specifically, any of the samples disclosed by the invention as detailed herein before in connection with other aspects.

In some embodiments, the kit of the invention is applicable for detecting any pathogen. It should be understood that any pathogen disclosed by the invention is also applicable for any of the kits of the invention. According to some specific embodiments, such pathogen may be a viral pathogen.

In yet some further embodiments, the kit of the invention may be applicable for detecting a viral pathogen in a sample, such viral pathogen in accordance with some embodiments may be at least one corona virus (CoV).

Specific embodiments of the invention relate to kits adapted for detecting a CoV that is SARS CoV-2. The kits of the invention are applicable for detecting pathogens in a sample. In some embodiments, specifically where the sample is obtained from a subject, particularly, a mammalian subject, the kit may be useful for the diagnosis and/or monitoring of at least one infectious disease caused by at least one pathogen in a subject. In some embodiments, such subject may be a mammalian subject.

In some embodiments, the kit of the invention is adapted for diagnosis and/or monitoring of at least one infectious disease in a subject, performed using any of the methods of the invention, specifically as described in connection with other aspects of the invention.

In some particular and non-limiting embodiments, the kits of the invention may be applicable and therefore adapted for the diagnosis and/or monitoring of COVID-19 in a mammalian subject.

In some embodiments, the kit of the invention may be comprised within a device. In more specific embodiments, the kit is comprised within and/or adapted for use in any of the devices of the invention, specifically, as defined by the invention. In yet some further embodiments, the kits of the invention may be comprised and/or adapted for use in any of the systems disclosed by the invention.

Still further, in some aspect thereof, the present disclosure further provides a therapeutic method that includes a diagnostic step. More specifically, the invention provides a method for treating and preventing a pathologic disorder in a subject in need caused by at least one pathogen. The method comprises the steps of: First step (a), involves contacting a sample of the subject or at least one aliquot thereof with an effective amount of at least one sample preparation agent/s (e.g., protease/s), to obtain at least one prepared sample. The next step (b) involves subjecting the at least one prepared sample of (a), or at least one aliquot thereof to at least one amplification reaction under isothermal conditions suitable for the production of at least one amplification product detectable by a detectable signal. It should be noted that at least one of such amplification reaction/s may be performed using at least one set of primers specific for at least one nucleic acid sequence of the nucleic acid sequence of interest. It should be noted that in some further embodiments, the method of the invention may further comprise in at least one of steps (a) and (b), contacting the sample with at least one chaotropic agent. More specifically, the at least one cheotropic agent may be contacted with the sample in step (a), during the preparation and incubation with the at least one protease. Alternatively, the at least one cheotropic agent may be contacted with the examined sample during the amplification reaction. In yet some further embodiments, the at least one cheotropic agent may be added to both steps, during the preparation of the sample in step (a) and during the amplification reaction in step (b). It should be noted that a detection of a detectable signal indicates the presence of the at least one pathogen in the examined sample, and thereby indicates that the subject is affected by the pathogen. The next step involves administration of a therapeutically effective amount of at least one anti-pathogenic agent to the subject. In some embodiments, the pathogen is a viral pathogen. In yet some further embodiments, the viral pathogen may be at least one CoV, specifically, SARS CoV-2. Thus, the present disclosure provides a therapeutic method for treating Covid-2019. The method comprises the step of applying the diagnostic method disclosed herein on at least one sample of a subject, and administering to subject classified as SARS CoV-2 positive, an effective amount of an anti-viral drug, or any other therapeutic agent.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%. The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Experimental Procedures

Samples Collection:

Swabs from both throat and nose were previously collected to one tube by healthcare providers and sent to the Virology laboratory at the Rambam Health Care Campus, Haifa, Israel. The swabs were stored in 1-2 ml of Universal Transport Medium (UTM). Saliva samples were self-collected directly into sterile cups and kept at 4° C. until tested.

Quantitative Reverse Transcription PCR (RT-qPCR):

Viral RNA was extracted by either of three automated nucleic acid extraction systems: (1) easyMAG®/EMAG (Biomeriuex); (2) magLEAD® 5bL (Precision System Science); (3) MagEx (STARlet) using the following protocols: (1) 2 ml lysis buffer, 0.5 ml sample and 50 μl elution buffer; (2) 270 μl lysis buffer, 130 μl sample and 50 μl elution buffer; (3) 300 μl lysis buffer, 400 μl sample and 50 μl elution buffer, respectively. Following viral RNA extraction, RT-qPCR was performed using either of two commercial kits: (1) Allplex™ 2019-nCoV (Seegene); (2) Real-Time Fluorescent RT-PCR Kit for Detecting SARS-2019-nCoV (BGI), according to manufacturer's instructions. Additional RT-qPCR reaction mix was created manually, using custom made primers (Primers table) as follows: The probe IC was synthesized with a 5′ FAM/CY5 Fluorophore moiety and 3′ ZEN/IBFQ quencher. Each of the manual reactions was assembled in a total volume of 25 μl. 12.5 μl 2×Ag-Path One-step mix (Ambion), 1 μl primers, 1 μl of Reverse Transcriptase enzyme, 5 μl of the sample's RNA extracted previously and H₂O to a final volume of 25 μl. All RT-qPCR reactions were executed in either of two systems: (1) Quantstudio (Thermo Fisher Scientific Inc.) or (2) CFX96 RealTime PCR machines (Bio-Rad) under the following conditions: 30 minutes at 50° C., 10 minutes at 95° C. and 45 cycles of two-step incubations: (1) 15 seconds at 95° C.; (2) 30 seconds at 55° C. Fluorescence was measured during step 2 of each cycle.

TABLE 1
Primer table (sequences of the specified primers used
herein are denoted by SEQ ID NO: 1 to 18).
GeneN-A primers published in Zhang et al., (2020) [4].
RNase P POP7 primers published in Curtis et al., (2018) [10].
RT-qPCR primers

| | | Final conc. | SEQ |
|---|---|---|---|
| Primer Name | Sequence | [nM] | ID NO: |
| E_Sarbeco_R2 | ATATTGCAGCAGTACGCACACA | 400 | |
| E_Sarbeco_P1 | ACACTAGCCATCCTTACTGCGCTTCG | 200 | 2 |
| E_Sarbeco_F1 | ACAGGTACGTTAATAGTTAATAGCGT | 400 | 3 |
| Primer FW IC | CATGGGAAGCAAGGGAACTAATG | 250 | 4 |
| (Upstream/1/Fw) | | | |
| Primer RV IC | CCCAGCGAGCAATACAGAATTT | 250 | 5 |
| (Downstream/2/Rv) | | | |
| Probe IC | 5′-CY5-TCTTCCCTCGAACCTGCACCATCAAT-3′ | 250 | 6 |

RT-LAMP primers

| | | Final conc. | |
|---|---|---|---|
| Primer Name | Sequence | [nM] | |
| GeneN-A-F3 | TGG CTA CCG AAG AGC T | 200 | 7 |
| GeneN-A-B3 | TGC AGC ATT GTT AGC AGG AT | 200 | 8 |
| GeneN-A-LF (Loop | GGA CTG AGA TCT TTC ATT TTA CCG T | 400 | 9 |
| Forward) | | | |
| GeneN-A-LB (Loop | ACT GAG GGA GCC TTG AAT ACA | 400 | 10 |
| Backward) | | | |
| GeneN-A-FIP | TCT GGC CCA GTT CCT AGG | 1600 | 11 |
| (Forward Inner | TAG TCC AGA CGA ATT CGT GGT GG | | |
| Primer) | | | |
| GeneN-A-BIP | AGA CGG CAT ATG GGT TGC | 1600 | 12 |
| (Backward Inner | ACG GGT GCC AAT GTG ATC T | | |
| Primer) | | | |
| RNaseP POP7 F3 | TTGATGAGCTGGAGCCA | 200 | 13 |
| RNaseP POP7 B3 | CACCCTCAATGCAGAGTC | 200 | 14 |
| RNaseP POP7 LF | ATGTGGATGGCTGAGTTGTT | 400 | 15 |
| RNaseP POP7 LB | CATGCTGAGTACTGGACCTC | 400 | 16 |
| RNaseP POP7 FIP | GTGTGACCCTGAAGACTCGGTTTTAGCCACTGACTC | 1600 | 17 |
| | GGATC | | |
| RNaseP POP7 BIP | CCTCCGTGATATGGCTCTTCGTTTTTTTCTTACATG | 1600 | 18 |
| | GCTCTGGTC | | |

Colorimetric RT-LAMP Reaction:

5 μl of UTM samples were diluted in 40 μl of DNase RNase free water (Biological Industries, 01-869-1B) and 2 μl of Proteinase K (1.22 mg/ml final concentration) (Seegene, 744300.4.UC384). Samples were incubated at room temperature for 15 minutes. Inactivation of proteinase K was obtained by incubating the reaction tubes at 95° C. for 5 minutes. Next, colorimetric RT-LAMP reaction was performed in a total volume of 20 μl per reaction using 10 μl WarmStart® Colorimetric LAMP 2× Master Mix (New England BioLabs Inc., M1800), 2 μl primers mix (see Primers Table), 1 μl Guanidine hydrochloride (Sigma, G4505), to a final concentration of 40 mM, and 7 μl of the inactivated sample. The reaction was then incubated for 30-40 minutes at 65° C. The proteinase K inactivation step and the reaction step were performed in a closed lid heating-block or, for a proof of concept, in a thermos-cup, using warm water. In the case of a thermos-cup, temperature was monitored by a simple thermometer. After 20 minutes of incubation, samples were monitored for color change every 5 minutes, by the naked eye, until reaching 40 min incubation. Samples were considered negative if the original pink color of the phenol red was maintained and positive if the phenol red color turned orange-yellow.

For the calibration process, reactions were performed with or without proteinase K and guanidine hydrochloride. Test samples were interpreted without prior knowledge of the reference standard results.

Statistical Analysis:

TPR (true positive rate), TNR (true negative rate), FPR (false positive rate), FNR (false negative rate) were calculated according to the following equations: TPR=TP/(TP+FN). TNR=TN/(FP+TN). FNR=FN/(TP+FN). FPR=FP/(FP+TN). TP: total number of true positives. TN: total number of true negatives. TN: total number of true negatives. FN: total number of false negatives.

Ethical Approval:

This study was granted exemption from IRB approval of the Rambam Health Care Campus for use of de-identified COVID-19 tests performed for the purpose of the standard testing, and for 4 volunteers.

### Example 1

Optimization of RT-LAMP for the Detection of SARS-CoV-2 RNA Directly from Crude Human Patient Swab Samples, without RNA Purification Steps

The inventors first performed RT-LAMP on RNA purified from COVID-19-positive and -negative swabs. The primers used in the present examples, were previously designed and validated by Zhang et al. [4] (see primers in Table 1). Samples were studied from positive and negative patients for the SARS-CoV-2. The samples were confirmed by approved RNA purification and quantification at the Rambam Health Care Campus (RHCC) hospital.

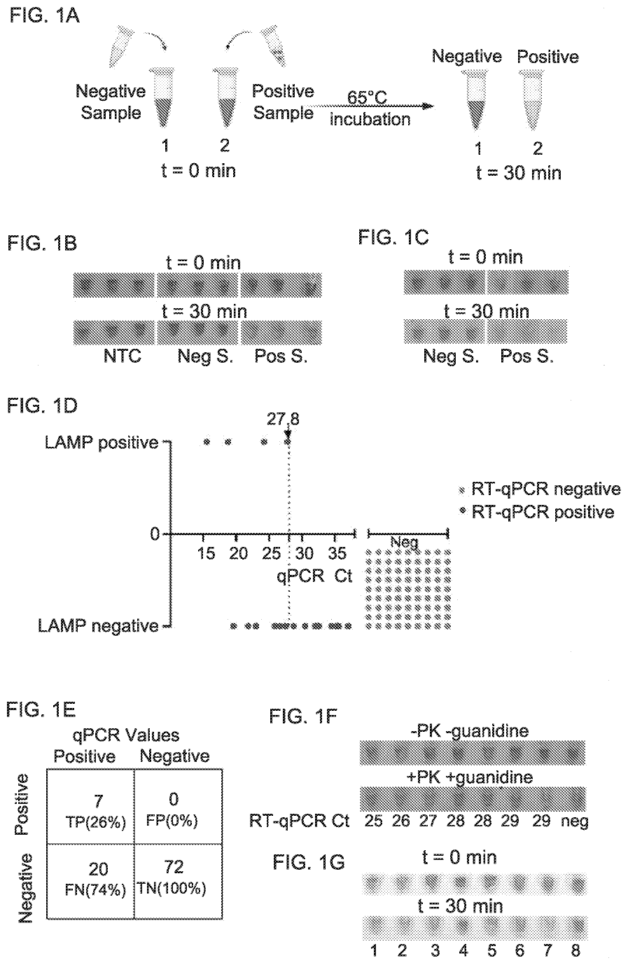

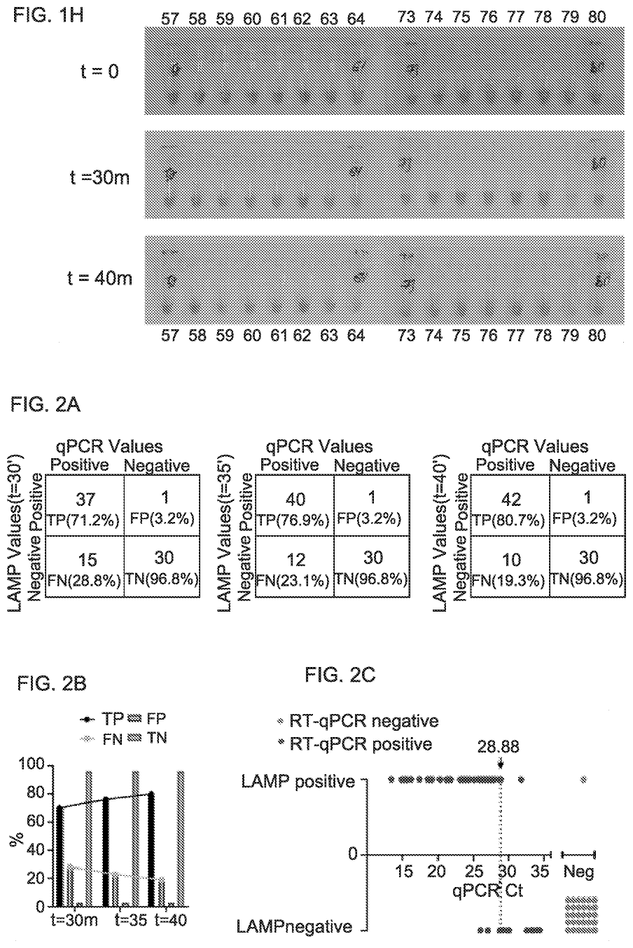

LAMP results can be visualized by color change (for graphical presentation see FIG. 1A). RT-LAMP was first applied on purified RNA from COVID-19 positive and negative swabs. As in Zhang et. al., [4], the RT-LAMP results agreed with the standard RT-qPCR results (FIG. 1B). To simplify the detection method, the RT-LAMP reaction was tested on crude throat and nose swabs from patients. These swabs were kept in universal transfer media (UTM). For crude samples, an inactivation step was added by heating the UTM to 95° C. for 5 minutes. Inactivated samples from confirmed patients were found to be positive by the RT-LAMP reaction (FIG. 1C). This RT-LAMP protocol was then evaluated on a cohort of 99 patients that were tested at the hospital. This pool included 27 positive samples with a wide range of viral load and 72 negative samples. Samples were previously evaluated by the standard RT-qPCR test. The detection limit of this RT-LAMP protocol was at cycle threshold (Ct) of 27.8 (FIG. 1D-1H, Table 2), with 7 true positives (TP), 20 false negatives (FN), 72 true negatives (TN), and no false positives (FP) (FIG. 1E, Table 2). Although there were no false positives, the rate of TP was very low. Hence, the RT-LAMP protocol of detection was improved from crude patient swabs. Since crude samples may contain enzymatic inhibitors that might affect the efficiency of viral RNA detection, the addition of proteinase K and guanidine hydrochloride to the process was tested. Proteinase K was added to a crude UTM sample taken from the original tube, before the inactivation step, and guanidine hydrochloride to the RT-LAMP reaction step. The two protocols were first compared on eight patients with low, medium and high Cts. Out of seven patients with positive results of RT-qPCR (Ct<37), two were clearly RT-LAMP positive in the former protocol and four in the new protocol. It was concluded that these adjustments improved the RT-LAMP efficiency of viral RNA detection directly from crude samples (FIG. 1F). Last, but not least, it was of interest to evaluate the specificity of this method. To this end, samples from patients confirmed to be infected with viruses other than SARS-CoV-2 were tested. Patients that were previously confirmed to be infected by other viruses (HSV, RSV, Influenza and Enterovirus) were tested. As shown in FIG. 1G, only the RNA of SARS-CoV-2 was detected positive.

TABLE 2
Detailed Ct threshold obtained for each sample

| | | | | | Test | Test |
|---|---|---|---|---|---|---|
| | | | | | results | results |
| | | | | | t(30) min | t(40) min |
| | | | | | (0- | (0- |
| | | | | | negative, | negative, |
| Sample | Sample | | RT-PCR | | 1- | 1- |
| # | type | Ct | result | Swab buffer | positive) | positive) |
| 1 | Swab | − | 0 | Serological tube red cup Pink | 0 | 0 |
| | | | | buffer | | |
| 2 | Swab | − | 0 | Serological tube red cup Pink | 0 | 0 |
| | | | | buffer | | |
| 3 | Swab | − | 0 | Serological tube red cup Pink | 0 | 0 |
| | | | | buffer | | |
| 4 | Swab | − | 0 | Serological tube red cup Pink | 0 | 0 |
| | | | | buffer | | |
| 5 | Swab | − | 0 | Serological tube red cup Pink | 0 | 0 |
| | | | | buffer | | |
| 6 | Swab | − | 0 | Serological tube red cup Pink | 0 | 0 |
| | | | | buffer | | |
| 7 | Swab | − | 0 | Serological tube red cup Pink | 0 | 0 |
| | | | | buffer | | |
| 8 | Swab | − | 0 | Serological tube red cup Pink | 0 | 0 |
| | | | | buffer | | |
| 9 | Swab | − | 0 | Serological tube red cup Pink | 0 | 0 |
| | | | | buffer | | |
| 10 | Swab | − | 0 | Serological tube red cup Pink | 0 | 0 |
| | | | | buffer | | |
| 11 | Swab | − | 0 | Serological tube red cup Pink | 0 | 0 |
| | | | | buffer | | |
| 12 | Swab | − | 0 | Serological tube red cup Pink | 0 | 0 |
| | | | | buffer | | |
| 13 | Swab | − | 0 | Serological tube red cup Pink | 0 | 0 |
| | | | | buffer | | |
| 14 | Swab | − | 0 | Serological tube red cup Pink | 0 | 0 |
| | | | | buffer | | |
| 15 | Swab | − | 0 | Serological tube red cup Pink | 0 | 0 |
| | | | | buffer | | |
| 16 | Swab | − | 0 | Serological tube red cup Pink | 0 | 0 |
| | | | | buffer | | |
| 17 | Swab | − | 0 | Serological tube red cup Pink | 0 | 0 |
| | | | | buffer | | |
| 18 | Swab | − | 0 | Serological tube red cup Pink | 0 | 0 |
| | | | | buffer | | |
| 19 | Swab | − | 0 | Serological tube red cup Pink | 0 | 0 |
| | | | | buffer | | |
| 20 | Swab | − | 0 | Serological tube red cup Pink | 0 | 0 |
| | | | | buffer | | |
| 21 | Swab | − | 0 | Serological tube red cup Pink | 0 | 0 |
| | | | | buffer | | |
| 22 | Swab | − | 0 | Serological tube red cup Pink | 0 | 0 |
| | | | | buffer | | |
| 23 | Swab | − | 0 | Serological tube red cup Pink | 0 | 0 |
| | | | | buffer | | |
| 24 | Swab | − | 0 | Serological tube red cup Pink | 0 | 0 |
| | | | | buffer | | |
| 25 | Swab | − | 0 | Serological tube red cup Pink | 0 | 0 |
| | | | | buffer | | |
| 26 | Swab | − | 0 | Serological tube red cup Pink | 0 | 0 |
| | | | | buffer | | |
| 27 | Swab | − | 0 | Serological tube red cup Pink | 0 | 0 |
| | | | | buffer | | |
| 28 | Swab | − | 0 | Serological tube red cup Pink | 0 | 0 |
| | | | | buffer | | |
| 29 | Swab | − | 0 | Serological tube red cup Pink | 0 | 0 |
| | | | | buffer | | |
| 30 | Swab | − | 0 | Serological tube red cup Pink | 0 | 0 |
| | | | | buffer | | |
| 31 | Swab | − | 0 | Serological tube red cup Pink | 0 | 0 |
| | | | | buffer | | |
| 32 | Swab | − | 0 | Serological tube red cup Pink | 0 | 0 |
| | | | | buffer | | |
| 33 | Swab | − | 0 | Serological tube red cup Pink | 0 | 0 |
| | | | | buffer | | |
| 34 | Swab | − | 0 | Serological tube red cup Pink | 0 | 0 |
| | | | | buffer | | |
| 35 | Swab | − | 0 | Serological tube red cup Pink | 0 | 0 |
| | | | | buffer | | |
| 36 | Swab | − | 0 | Serological tube red cup Pink | 0 | 0 |
| | | | | buffer | | |
| 37 | Swab | − | 0 | Serological tube red cup Pink | 0 | 0 |
| | | | | buffer | | |
| 38 | Swab | − | 0 | Serological tube red cup Pink | 0 | 0 |
| | | | | buffer | | |
| 39 | Swab | − | 0 | Serological tube red cup Pink | 0 | 0 |
| | | | | buffer | | |
| 40 | Swab | − | 0 | Serological tube red cup Pink | 0 | 0 |
| | | | | buffer | | |
| 41 | Swab | − | 0 | Serological tube red cup Pink | 0 | 0 |
| | | | | buffer | | |
| 42 | Swab | − | 0 | Serological tube red cup Pink | 0 | 0 |
| | | | | buffer | | |
| 43 | Swab | − | 0 | Serological tube red cup Pink | 0 | 0 |
| | | | | buffer | | |
| 44 | Swab | − | 0 | Serological tube red cup Pink | 0 | 0 |
| | | | | buffer | | |
| 45 | Swab | − | 0 | Serological tube red cup Pink | 0 | 0 |
| | | | | buffer | | |
| 46 | Swab | − | 0 | Serological tube red cup Pink | 0 | 0 |
| | | | | buffer | | |
| 47 | Swab | − | 0 | Serological tube red cup Pink | 0 | 0 |
| | | | | buffer | | |
| 48 | Swab | − | 0 | Serological tube red cup Pink | 0 | 0 |
| | | | | buffer | | |
| 49 | Swab | − | 0 | Serological tube red cup Pink | 0 | 0 |
| | | | | buffer | | |
| 50 | Swab | − | 0 | Serological tube red cup Pink | 0 | 0 |
| | | | | buffer | | |
| 51 | Swab | − | 0 | Serological tube red cup Pink | 0 | 0 |
| | | | | buffer | | |
| 52 | Swab | − | 0 | Serological tube red cup Pink | 0 | 0 |
| | | | | buffer | | |
| 53 | Swab | − | 0 | Serological tube red cup Pink | 0 | 0 |
| | | | | buffer | | |
| 54 | Swab | − | 0 | Serological tube red cup Pink | 0 | 0 |
| | | | | buffer | | |
| 55 | Swab | − | 0 | Serological tube red cup Pink | 0 | 0 |
| | | | | buffer | | |
| 56 | Swab | − | 0 | Serological tube red cup Pink | 0 | 0 |
| | | | | buffer | | |
| 57 | Swab | − | 0 | Serological tube red cup Pink | 0 | 0 |
| | | | | buffer | | |
| 58 | Swab | − | 0 | Serological tube red cup Pink | 0 | 0 |
| | | | | buffer | | |
| 59 | Swab | − | 0 | 15 ml falcon orange cup clear | 0 | 0 |
| | | | | buffer | | |
| 60 | Swab | − | 0 | 15 ml falcon orange cup clear | 0 | 0 |
| | | | | buffer | | |
| 61 | Swab | − | 0 | 15 ml falcon orange cup clear | 0 | 0 |
| | | | | buffer | | |
| 62 | Swab | − | 0 | 15 ml falcon orange cup clear | 0 | 0 |
| | | | | buffer | | |
| 63 | Swab | − | 0 | Serological tube red cup Pink | 0 | 0 |
| | | | | buffer | | |
| 64 | Swab | + | 1 | Serological tube blue cup | 1 | 1 |
| | | | | yellow buffer | | |
| 65 | Swab | + | 1 | Serological tube red cup Pink | 1 | 1 |
| | | | | buffer | | |
| 66 | Swab | 35.22 | 1 | Serological tube blue cup | 0 | 0 |
| | | | | yellow buffer | | |
| 67 | Swab | − | 0 | Falcon 15 ml Blue cap | 0 | 0 |
| | | | | reddish buffer | | |
| 68 | Swab | 25.87 | 1 | Falcon 15 ml Blue cap | 0 | 0 |
| | | | | reddish buffer | | |
| 69 | Swab | 18.76 | 1 | Falcon 15 ml Blue cap | 1 | 1 |
| | | | | reddish buffer | | |
| 70 | Swab | − | 0 | Falcon 15 ml Blue cap | 0 | 0 |
| | | | | reddish buffer | | |
| 71 | Swab | + | 1 | 15 ml falcon orange cup clear | 0 | 0 |
| | | | | buffer | | |
| 72 | Swab | + | 1 | 15 ml falcon orange cup clear | 1 | 1 |
| | | | | buffer | | |
| 73 | Swab | + | 1 | Falcon 15 ml Blue cap | 0 | 0 |
| | | | | reddish buffer | | |
| 74 | Swab | 34.46 | 1 | Falcon 15 ml Blue cap | 0 | 0 |
| | | | | reddish buffer | | |
| 75 | Swab | 32.31 | 1 | 15 ml falcon orange cup clear | 0 | 0 |
| | | | | buffer | | |
| 76 | Swab | 27.51 | 1 | Falcon 15 ml Blue cap | 0 | 0 |
| | | | | reddish buffer | | |
| 77 | Swab | 19.58 | 1 | Serological tube red cup Pink | 0 | 0 |
| | | | | buffer | | |
| 78 | Swab | − | 0 | Serological tube red cup Pink | 0 | 0 |
| | | | | buffer | | |
| 79 | Swab | 15.51 | 1 | 15 ml falcon orange cup clear | 1 | 1 |
| | | | | buffer | | |
| 80 | Swab | − | 0 | 15 ml falcon orange cup clear | 0 | 0 |
| | | | | buffer | | |
| 81 | Swab | 24.22 | 1 | Serological tube red cup Pink | 1 | 1 |
| | | | | buffer | | |
| 82 | Swab | 30.47 | 1 | 15 ml falcon orange cup clear | 0 | 0 |
| | | | | buffer | | |
| 83 | Swab | 21.8 | 1 | Serological tube red cup Pink | 0 | 0 |
| | | | | buffer | | |
| 84 | Swab | 23 | 1 | Serological tube red cup Pink | 0 | 0 |
| | | | | buffer | | |
| 85 | Swab | 27.8 | 1 | Serological tube red cup Pink | 1 | 1 |
| | | | | buffer | | |
| 86 | Swab | 37 | 1 | Serological tube red cup Pink | 0 | 0 |
| | | | | buffer | | |
| 87 | Swab | 32 | 1 | Serological tube red cup Pink | 0 | 0 |
| | | | | buffer | | |
| 88 | Swab | 26.72 | 1 | Serological tube red cup Pink | 0 | 0 |
| | | | | buffer | | |
| 89 | Swab | − | 0 | Serological tube red cup Pink | 0 | 0 |
| | | | | buffer | | |
| 90 | Swab | 35.6 | 1 | Falcon 15 ml Blue cap | 0 | 0 |
| | | | | reddish buffer | | |
| 91 | Swab | 32.6 | 1 | Serological tube red cup Pink | 0 | 0 |
| | | | | buffer | | |
| 92 | Swab | 31.95 | 1 | Serological tube red cup Pink | 0 | 0 |
| | | | | buffer | | |
| 93 | Swab | − | 0 | Serological tube red cup Pink | 0 | 0 |
| | | | | buffer | | |
| 94 | Swab | − | 0 | Serological tube red cup Pink | 0 | 0 |
| | | | | buffer | | |
| 95 | Swab | − | 0 | Serological tube red cup Pink | 0 | 0 |
| | | | | buffer | | |
| 96 | Swab | 35.3 | 1 | Serological tube red cup Pink | 0 | 0 |
| | | | | buffer | | |
| 97 | Swab | 26.7 | 1 | Serological tube red cup Pink | 0 | 0 |
| | | | | buffer | | |
| 98 | Swab | 28.72 | 1 | Serological tube red cup Pink | 0 | 0 |
| | | | | buffer | | |
| 99 | Swab | − | 0 | Falcon 15 ml Blue cap | 0 | 0 |
| | | | | reddish buffer | | |

### Example 2

Validation Analysis an Additional Cohort of Patients Suspected of SARS-CoV-2

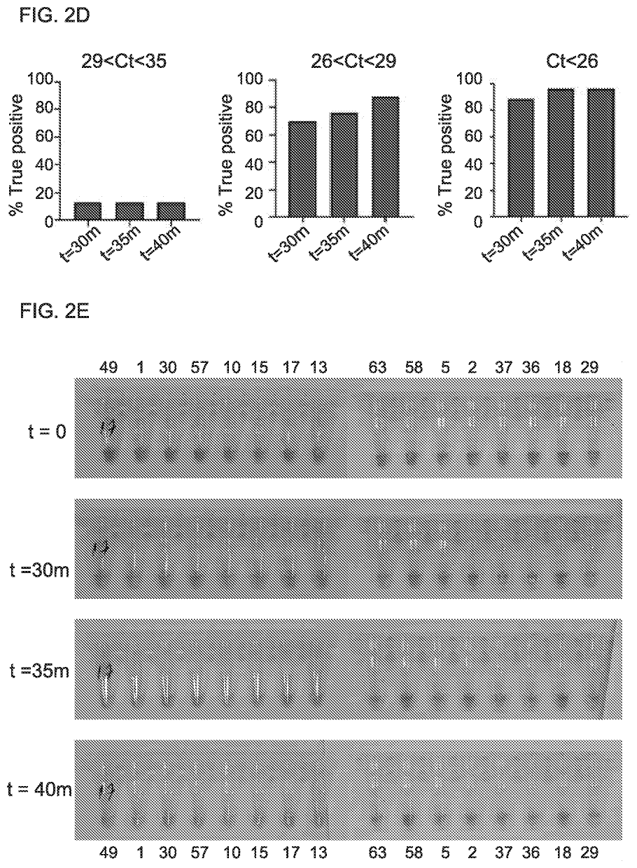

This adjusted protocol was further validated on an additional cohort of 83 patients suspected of SARS-CoV-2. These patients were tested at RHCC by the standard RT-qPCR, 31 were negative and 52 were positive with a wide range of Ct values (14-35). It was of interest to find the optimal incubation time to yield the best rate of true positives without increasing the rate of false negatives. The RT-LAMP reaction was performed for up to 40 minutes and evaluated the colorimetric results at time-points 30, 35 and 40 minutes. With time, the number of TP increased, the number of FN decreased, with no change in the numbers of TN and with one FP throughout (FIG. 2A-2B, Table 3). Time-points 35 and 40 showed the highest TP rate in samples with low (Ct<26) and medium (26<Ct<29) Ct values (FIG. 2D-2E, Table 3). Hence, in these conditions, time-points 35 and 40 were better altogether. FIG. 2C shows the results of the adjusted method at time point 40. These results were compared to the RT-qPCR Ct values of the same patients. RT-LAMP was most sensitive in detection of positive patients with viral load that corresponds to low and medium Ct values. Under Ct 28.8, the true positive rate of RT-LAMP was 93%.

TABLE 3
Detailed Ct threshold for each sample

| | | | | Test | Test | Test |
|---|---|---|---|---|---|---|
| | | | | results | results | results |
| | | | | t(30) | t(35) min | t(40) min |
| | | | | min (0- | (0- | (0- |
| | | | | negative, | negative, | negative, |
| Sam- | Sample | | RT-PCR | 1- | 1- | 1- |
| ple# | type | CT | result | positive) | positive) | positive) |
| 1 | swab | 26.48 | 1 | 1 | 1 | 1 |
| 2 | swab | 28.02 | 1 | 1 | 1 | 1 |
| 3 | swab | 30.07 | 1 | 0 | 0 | 0 |
| 4 | swab | 32.64 | 1 | 0 | 0 | 0 |
| 5 | swab | 19.2 | 1 | 1 | 1 | 1 |
| 6 | swab | 23.18 | 1 | 1 | 1 | 1 |
| 7 | swab | 28.7 | 1 | 1 | 1 | 1 |
| 8 | swab | 17.38 | 1 | 1 | 1 | 1 |
| 9 | swab | 25.06 | 1 | 0 | 1 | 1 |
| 10 | swab | 24.43 | 1 | 1 | 1 | 1 |
| 11 | swab | 14.9 | 1 | 1 | 1 | 1 |
| 12 | swab | 28.88 | 1 | 1 | 1 | 1 |
| 13 | swab | 27.26 | 1 | 1 | 1 | 1 |
| 14 | swab | 13.4 | 1 | 1 | 1 | 1 |
| 15 | swab | 27.03 | 1 | 0 | 1 | 1 |
| 16 | swab | 23.71 | 1 | 1 | 1 | 1 |
| 17 | swab | 27.08 | 1 | 0 | 0 | 0 |
| 18 | swab | 21.19 | 1 | 1 | 1 | 1 |
| 19 | swab | 18.77 | 1 | 1 | 1 | 1 |
| 20 | swab | 21.6 | 1 | 1 | 1 | 1 |
| 21 | swab | 20.25 | 1 | 1 | 1 | 1 |
| 22 | swab | 26.49 | 1 | 1 | 1 | 1 |
| 23 | swab | 25.87 | 1 | 0 | 1 | 1 |
| 24 | swab | 25.84 | 1 | 1 | 1 | 1 |
| 25 | swab | 15.26 | 1 | 1 | 1 | 1 |
| 26 | swab | 15.51 | 1 | 1 | 1 | 1 |
| 27 | swab | 21.44 | 1 | 1 | 1 | 1 |
| 28 | swab | 23.6 | 1 | 1 | 1 | 1 |
| 29 | swab | 15.22 | 1 | 1 | 1 | 1 |
| 30 | swab | 26.77 | 1 | 0 | 0 | 1 |
| 31 | swab | 27 | 1 | 1 | 1 | 1 |
| 32 | swab | 16.15 | 1 | 1 | 1 | 1 |
| 33 | swab | 27.57 | 1 | 1 | 1 | 1 |
| 34 | swab | 25.72 | 1 | 1 | 1 | 1 |
| 35 | swab | 24.03 | 1 | 1 | 1 | 1 |
| 36 | swab | 25.99 | 1 | 0 | 0 | 0 |
| 37 | swab | 26.01 | 1 | 1 | 1 | 1 |
| 38 | swab | 26.29 | 1 | 1 | 1 | 1 |
| 39 | swab | 23.49 | 1 | 1 | 1 | 1 |
| 40 | swab | 29.25 | 1 | 0 | 0 | 0 |
| 41 | swab | 29.84 | 1 | 0 | 0 | 0 |
| 42 | swab | 19.1 | 1 | 1 | 1 | 1 |
| 43 | swab | 28.81 | 1 | 0 | 0 | 0 |
| 44 | swab | 26.04 | 1 | 1 | 1 | 1 |
| 45 | swab | 18.55 | 1 | 1 | 1 | 1 |
| 46 | swab | 34.41 | 1 | 0 | 0 | 0 |
| 47 | swab | 33.96 | 1 | 0 | 0 | 0 |
| 48 | swab | 21.82 | 1 | 1 | 1 | 1 |
| 49 | swab | 31.82 | 1 | 1 | 1 | 1 |
| 50 | swab | 33.34 | 1 | 0 | 0 | 0 |
| 51 | swab | 27.91 | 1 | 1 | 1 | 1 |
| 57 | swab | 26.72 | 1 | 0 | 0 | 1 |
| 52 | swab | — | 0 | 0 | 0 | 0 |
| 53 | swab | — | 0 | 0 | 0 | 0 |
| 54 | swab | — | 0 | 0 | 0 | 0 |
| 55 | swab | — | 0 | 1 | 1 | 1 |
| 56 | swab | — | 0 | 0 | 0 | 0 |
| 58 | swab | — | 0 | 0 | 0 | 0 |
| 59 | swab | — | 0 | 0 | 0 | 0 |
| 60 | swab | — | 0 | 0 | 0 | 0 |
| 61 | swab | — | 0 | 0 | 0 | 0 |
| 62 | swab | — | 0 | 0 | 0 | 0 |
| 63 | swab | — | 0 | 0 | 0 | 0 |
| 64 | swab | — | 0 | 0 | 0 | 0 |
| 65 | swab | — | 0 | 0 | 0 | 0 |
| 66 | swab | — | 0 | 0 | 0 | 0 |
| 67 | swab | — | 0 | 0 | 0 | 0 |
| 68 | swab | — | 0 | 0 | 0 | 0 |
| 69 | swab | — | 0 | 0 | 0 | 0 |
| 70 | swab | — | 0 | 0 | 0 | 0 |
| 71 | swab | — | 0 | 0 | 0 | 0 |
| 72 | swab | — | 0 | 0 | 0 | 0 |
| 73 | swab | — | 0 | 0 | 0 | 0 |
| 74 | swab | — | 0 | 0 | 0 | 0 |
| 75 | swab | — | 0 | 0 | 0 | 0 |
| 76 | swab | — | 0 | 0 | 0 | 0 |
| 77 | swab | — | 0 | 0 | 0 | 0 |
| 78 | swab | — | 0 | 0 | 0 | 0 |
| 79 | swab | — | 0 | 0 | 0 | 0 |
| 80 | swab | — | 0 | 0 | 0 | 0 |
| 81 | swab | — | 0 | 0 | 0 | 0 |
| 82 | swab | — | 0 | 0 | 0 | 0 |
| 83 | swab | — | 0 | 0 | 0 | 0 |

### Example 3

Optimal RT-LAMP on Human Saliva Samples

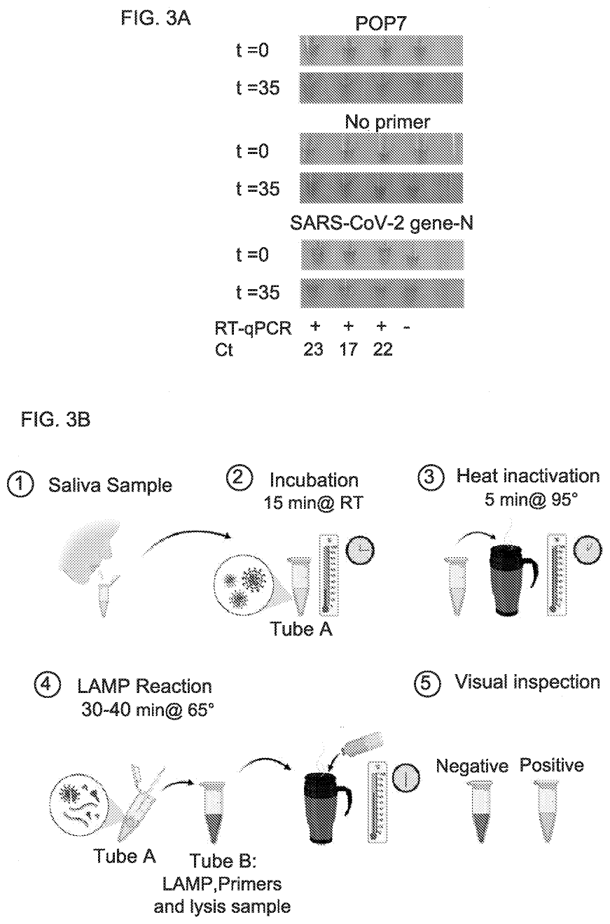

Human to human transmission of SARS-CoV-2 is mainly through saliva droplets [9]. A comparison between saliva samples to the standard swabs showed a higher viral load in the saliva [10]. Moreover, the FDA has recently approved saliva as a possible way of sampling for COVID-19. Therefore, RT-LAMP was next performed on human saliva samples. Saliva samples were self-collected from three different confirmed patients, and one suspected negative subject. In parallel, the standard RNA purification and RT-qPCR reactions were also performed in the hospital setting. The confirmed patients were found positive in both RT-LAMP and RT-qPCR from saliva, and the suspected negative subject was confirmed negative (FIG. 3A). Here, as a positive control for the reaction and saliva sampling, the human gene POP7 was tested, in addition to gene N of SARS-CoV-2. POP7 was detected in all saliva samples (FIG. 3A).

Due to the isothermal property of the reaction and the fact that the test is detectable in saliva samples, the reaction can potentially be performed in any constant heat source. Self-sampling can be done in a thermal-cup as shown in (FIG. 3B).

### Example 4

Self-Sampling Detection Kit

To implement this detection to the community, a self-sampling kit is produced. This kit is composed of two compartments. A box, like the box of ear pods with two compartments in it. One has a cap with a loop stick connected to it. The loop is inserted to the mouth and then into the tube (tube A). It should be however noted that other means for obtaining and inserting the sample into the sample preparation chamber are also used by the inventors. Non-limiting examples include a sponge, an absorbing matrix, or a gum that soak the saliva and then applied to the device. Then RNAse, DNase free water is inserted to the tube, dissolving the proteinase K. After 15 minutes at room temperature, the tube is turned and a small door opens up into the adjacent tube in which the reaction mix is ‘waiting’. A small amount of tube A is inserted to the reaction mix—temperature is set to 65° C., this part of the box has a window in which the reaction can be seen, the tube shows a color change to indicate the result. TUBE B (the second tube) is split to two—before the 65° C. Such that in one tube the primers show a positive technical control, and in the other—the reaction itself. At the end of the process—temperature of the whole apparatus rises to 95° C. for 10 minutes, and the content discarded. Alternatively, there is a sterilization material that washes the whole inside part of this apparatus.

### Example 5

Extended Validation Analysis—A Clinical Trial

The adjusted protocol disclosed by the present disclosure was further validated on additional cohorts of cases suspected of SARS-CoV-2, obtained from several medical centers. Participants in the study were of ages 18-65.

TABLE 4

| | |
|---|---|
| Accuracy | 93.85% in the infectious range (CT < 30) with the |
| | applicable samples. |
| Sensitivity | 1. frozen Saliva Test: Analysis was based on more |
| | than 350 defrosted frozen saliva samples from |
| | confirmed Positive patients (using NP Swabs), and a |
| | random defrosted Saliva sample from 2900 negative |
| | samples within the same study. Samples were |
| | collected in August 2020. |
| | Each Patient was tested by two PCRs and as 2/3 of |
| | the results were positive only in one institution, |
| | statistics are focused around the DP-Double |
| | Positives. We had further cross-referenced these |
| | samples with a Saliva qPCR measured and applied a |
| | filter of Ct < 30 of the saliva qPCR. |
| | The filter above left for analysis 65 Double Positive |
| | samples out of 128 DP. |
| | By using this filter, the sensitivity of our LAMP test |
| | was 93.85%. |
| | There were zero FP in our analysis of negative |
| | (unblinded) samples. |
| | Sensitivity of the sample with Saliva qPCR < 34 was |
| | 84%. |
| | There was NO difference in performance between |
| | symptomatic and asymptomatic confirmed cases. |
| | 2. Fresh Saliva Study (February 2021): |
| | 41 cases of which 8 confirmed positive with CT < 33 |
| | (lowest CT = 17). 100% sensitivity and 100% |
| | specificity. An additional sample of CT = 33 was |
| | inconclusive. |
| Specificity -1 | Distinguishing (in the lab) from other viruses and |
| | bacteria: 100% checked against the following |
| | common respiratory flora and viral pathogens viruses |
| | and bacteria: HSV; RSV; Influenza B; Enterovirus. |
| Specificity -2: True positive (Positive | 99.5% tested against lab PCR tests. The rate of |
| patient will have positive result) | positive answers with our test that were indeed |
| | confirmed positive with swab PCR test. |
| False negative (1-sensitivity) | 6% for CT < 30 (the infectious range). |
| False negative rate is the proportion of | |
| positives which yield negative test | |
| outcomes with the test, i.e., the | |
| conditional probability of a negative | |
| test result given that the condition | |
| being looked for is present. | |

1-40. (canceled)

41. Device for enabling detection of at least one nucleic acid sequence of interest in at least one sample, comprising a sample module and a reaction module, wherein:
the sample module is configured for accommodating therein a plurality of sample preparation agents including at least a sample to be tested, and for enabling mixing of said plurality of sample preparation agents to provide a prepared sample, said sample module being configured for selectively allowing insertion of the sample into the sample module, and further configured for enabling delivery of said prepared sample to the reaction module;
the reaction module comprises a plurality of reaction chambers, the device configured for selectively delivering a respective aliquot of said prepared sample to each said reaction chamber, wherein each said reaction chamber is configured for accommodating therein a respective quantity of a respective reaction mixture adapted for amplification reaction under isothermal conditions;
the device is further configured for enabling reacting of each said respective aliquot of said prepared sample with each said respective reaction mixture in the respective said reaction chamber to produce at least one amplification product; and
the device is further configured for enabling detecting a respective test parameter associated with said production of said at least one amplification product.

42. The device according to claim 41, wherein at least one of:
(a) said sample preparation agents comprise a first quantity of at least one protease, and a second quantity of a solubilizing liquid;
(b) said respective quantity of a respective reaction mixture is a respective third quantity of a respective reaction mixture adapted for amplification reaction under isothermal conditions; and
(c) at least one of said sample module and said reaction module further comprise a fourth quantity of at least one chaotropic agent, optionally, wherein the sample module comprises a sample module housing defining therein a sample chamber, the sample chamber being configured for accommodating therein said plurality of sample preparation agents and for enabling mixing of said plurality of sample preparation agents to provide said prepared sample.

43. The device according to claim 42, wherein said sample module is configured for accommodating said first quantity of at least one protease therein, prior to use of the device with a user, optionally, wherein said sample module is configured for enabling said second quantity of a solubilizing liquid to be selectively inserted into said sample chamber from an external source.

44. The device according to claim 42, wherein the sample chamber is configured for separately accommodating therein said plurality of sample preparation agents and for enabling selectively mixing of said plurality of sample preparation agents to provide said prepared sample, optionally, wherein said sample module comprises a first sub-chamber configured for accommodating said first quantity of at least one protease therein, prior to use of the device with a user, and a second sub-chamber, different from said first sub-chamber, wherein the second sub-chamber is configured for accommodating said second quantity of a solubilizing liquid therein, prior to use of the device with a user, and wherein said sample module comprises a third sub-chamber configured for accommodating the sample when the device is used with respect to a user.

45. The device according to claim 44, wherein said sample module is configured for selectively enabling fluid communication between said first sub-chamber, said second sub-chamber, and said third sub-chamber to enable mixing of said plurality of sample preparation agents to provide the prepared sample, optionally, wherein:
(a) said sample module comprises a membrane separating said second sub-chamber from said first sub-chamber and said second sub-chamber, and wherein said membrane is selectively rupturable to enable mixing of said plurality of sample preparation agents to provide the prepared sample; or
(b) wherein said sample module comprises a barrier separating said second sub-chamber from said first sub-chamber and said second sub-chamber, and wherein said barrier comprises a first valve member that is selectively openable to enable mixing of said plurality of sample preparation agents to provide the prepared sample.

46. The device according to claim 42, wherein said sample module comprises an inlet port for selectively allowing insertion of at least the sample therethrough and into the sample chamber, optionally, wherein
(a) wherein said sample module comprises a cap for selectively opening and closing said inlet port, and wherein said cap includes a sampling member projecting from an inner part of the cap such that when the cap closes said inlet port the sampling member is inside said sample chamber;
(b) wherein said sample module comprises a sampling member in the form of a cartridge that is selectively insertable into the sample chamber via said inlet port, and wherein said cartridge includes a sample surface onto which a user can deposit the sample thereon;
(c) wherein said sample module includes a sample surface spaced from said inlet port, and wherein the sample module is configured for enabling a user to deposit the sample onto said sample surface via the inlet port.

47. The device according to claim 42, wherein the sample module is configured for enabling delivery of said prepared sample to the reaction module from the sample chamber, optionally, wherein said sample chamber comprises an outlet port coupled to the reaction module, the outlet port being configured for selectively enabling fluid communication between the sample chamber and the reaction module to thereby enable delivery of said prepared sample to the reaction module from the sample chamber, optionally, the outlet port comprising a second valve member configured for selectively enabling fluid communication between the sample chamber and the reaction module to thereby enable delivery of said prepared sample to the reaction module from the sample chamber.

48. The device according to claim 42, wherein the device is configured for selectively separating the prepared sample into a plurality said aliquots of said prepared sample, and for directing each said aliquot to a respective said reaction chamber, optionally, wherein the reaction module comprises a manifold unit having an inlet opening in fluid communication with said outlet port, and a plurality of exit ports, each exit port being in fluid communication with a respective said reaction chamber.

49. The device according to claim 41, wherein at least one of:
(a) wherein each said reaction chamber is configured for accommodating therein said respective third quantity of said respective reaction mixture, prior to use of the device with a user;
(b) wherein said reaction module comprises at least two said reaction chambers, wherein a first said reaction chamber is configured for accommodating therein said respective third quantity of a respective first said reaction mixture, and wherein a second said reaction chamber is configured for accommodating therein a said respective third quantity of a respective second said reaction mixture, prior to use of the device with a user, optionally, wherein said first reaction mixture is configured for testing for production of at least one amplification product of a nucleic acid sequence of said pathogen, and wherein said second reaction mixture is configured for production of at least one amplification product of a control nucleic acid sequence, thereby providing a control test;
(c) wherein each said respective test parameter is in the form of a visually detectable specific color associated with respective reaction mixture subsequent to interaction of said respective aliquot of said prepared sample with the respective said reaction mixture in the respective said reaction chamber, optionally, wherein at least a part of the reaction module is transparent to allow each said specific color to be externally observed, recorded and/or quantified; and
(d) wherein each said respective test parameter is detectable as any one of a specific fluorescence parameter, a specific pH value, electric charge and conductivity, or production of pyrophosphate each being associated with respective reaction mixture subsequent to interaction of said respective aliquot of said prepared sample with the respective said reaction mixture in the respective said reaction chamber, optionally, wherein the reaction module is configured for enabling, for each reaction chamber, the respective said specific fluorescence parameter, the respective said specific pH value, or the respective said electric charge, or the respective conductivity, or the respective production of pyrophosphate to be externally detected, recorded and/or quantified.

50. The device according to claim 41, wherein at least one of:
(a) each said reaction chamber comprises therein said respective third quantity of said respective reaction mixture, and wherein said sample module comprises therein said first quantity of at least one protease, and wherein said device is configured for enabling selective delivery of said second quantity of a solubilizing liquid into said sample module, optionally, said device further comprising said fourth quantity of at least one chaotropic agent;
(b) each said reaction chamber comprises therein said respective third quantity of said respective reaction mixture, and wherein said sample module comprises therein said first quantity of at least one protease, and said second quantity of a solubilizing liquid, wherein said sample module is configured for initially maintaining said first quantity of at least one protease separate from said second quantity of a solubilizing liquid, and for selectively enabling mixing of said first quantity of at least one protease with said second quantity of a solubilizing liquid, optionally, said device further comprising said fourth quantity of at least one chaotropic agent;
(c) wherein at least one of:
(i) said at least one protease of said first quantity is proteinase K (PK);
(ii) said solubilizing liquid of said second quantity comprises water; and
(iii) said at least one chaotropic agent of said fourth quantity is guanidine hydrochloride; and
(d) wherein at least one of:
(i) said amplification reaction is a loop mediated isothermal amplification reaction (LAMP);
(ii) at least one of said third quantity of said at least one first reaction mixture comprises at least one set of primers specific for at least one nucleic acid sequence of said at least one nucleic acid sequence of interest, optionally, at least one of said third quantity of said at least one second reaction mixture comprises at least one set of primers specific for at least one control nucleic acid sequence;
(iii) wherein said sample is at least one of a biological sample and an environmental sample; and
(e) wherein said nucleic acid sequence of interest is a nucleic acid sequence of at least one pathogen, optionally, at least one of:
(i) said pathogen is a viral pathogen;
(ii) said pathogen is a viral pathogen, and wherein said viral pathogen is at least one corona virus (CoV); and
(iii) said pathogen is a CoV, wherein said CoV is Severe acute respiratory syndrome (SARS) CoV-2.

51. A system for detecting at least one nucleic acid sequence of interest in at least one sample, comprising:
at least one device as defined in claim 50;
a heating apparatus configured for heating at least said reaction module to a predetermined range of temperatures above ambient, optionally, wherein said heating apparatus is configured for being coupled to the device and comprises a heating system configured for directing heat to the device when coupled thereto, optionally, any one of:
(a) wherein predetermined range of temperatures includes a temperature of 65° C.+/−5° C.; or
(b) wherein predetermined range of temperatures includes a temperature of 95° C.+/−5° C.

52. The system according to claim 51, further comprising detection apparatus configured for detecting said test parameters associated with each of the respective said reaction chambers, optionally, any one of:
(a) wherein test parameter for each respective said test chamber is a respective said specific color, and wherein the detection apparatus is configured for determining a respective wavelength of the respective said specific color;
(b) wherein test parameter for each respective said test chamber is a respective said specific fluorescence parameter, and wherein the detection apparatus is configured for determining a respective fluorescence value of the respective said specific fluorescence parameter; or
(c) wherein test parameter for each respective said test chamber is a respective said specific pH parameter, and wherein the detection apparatus is configured for determining a respective pH value of the respective said specific pH parameter.

53. A method for the detection and monitoring of at last one nucleic acid sequence of interest in at least one sample, the method comprising the steps of:
(a) contacting said sample or at least one aliquot thereof with an effective amount of at least one sample preparation agent to obtain at least one prepared sample; and
(b) subjecting the at least one prepared sample of (a), or at least one aliquot thereof to at least one amplification reaction under isothermal conditions suitable for the production of at least one amplification product detectable by a detectable signal; wherein at least one of said amplification reaction/s is performed using at least one set of primers specific for at least one nucleic acid sequence of said nucleic acid sequence of interest;
wherein the detection of a detectable signal indicates the presence of said nucleic acid sequence of interest in said sample, optionally, wherein said step (b) further comprises subjecting at least one aliquot of the prepared sample of (a) to at least one amplification reaction using at least one set of primers specific for at least one control nucleic acid sequence.

54. The method according to claim 53, wherein at least one of:
(i) said at least one sample preparation agent comprise at least one protease; and
(ii) said at least one of steps (a) and (b) further comprise contacting the sample with at least one chaotropic agent, optionally, wherein at least one of,
(a) said amplification reaction is a loop mediated isothermal amplification reaction (LAMP), and wherein said LAMP reaction is performed using a reaction mixture comprising a pH sensitive indicator dye providing a detectable signal upon production of at least one amplification product, optionally, one of: (i) said pH sensitive indicator dye is a colored dye detectable in visible light; or (ii) said pH sensitive indicator dye is a fluorescent indicator dye;
(b) said LAMP reaction is performed using a reaction mixture comprising at least one detectable compound capable of intercalating into a double strand DNA, thereby providing a detectable signal upon production of at least one amplification product;
(c) said sample is at least one of a biological sample and an environmental sample; and
(d) said at least one nucleic acid sequence of interest is at least one nucleic acid sequence of at least one pathogen.

55. The method according to claim 54, wherein said pathogen is a viral pathogen, optionally, at least one of:
(a) said viral pathogen is at least one corona virus (CoV); and
(b) said CoV is Severe acute respiratory syndrome (SARS) CoV-2.

56. The method according to claim 53, for the diagnosis of an infectious disease caused by at least one of said pathogen in a subject, optionally, for the diagnosis of COVID-19 in a mammalian subject.

57. A kit comprising:
(a) an effective amount of at least one protease, optionally comprised within a sample preparation module;
(b) an amplification reaction mixture, optionally comprised within a reaction module that comprises a plurality of reaction chambers, each of said reaction chambers comprises said amplification mixture; wherein said reaction mixture is adapted for amplification reaction under isothermal conditions, and wherein said reaction mixture in at least one first said reaction chamber comprises at least one set of primers specific for at least one nucleic acid sequence of at least one pathogen;
(c) at least one chaotropic agent comprised within at least one of the sample preparation module (a) and the reaction module (b), optionally,
wherein said reaction mixture in at least one second said reaction chamber comprises at least one set of primers specific for at least one control nucleic acid sequence.

58. The kit according to claim 57, wherein at least one of:
(a) said amplification reaction is LAMP, and wherein said reaction mixture further comprises at least one pH sensitive indicator dye, said dye provides a detectable signal upon production of at least one amplification product by said amplification reaction, optionally, one of: (i) wherein said pH sensitive indicator dye is a colored dye detectable in visible light; or (ii) wherein said pH sensitive indicator dye is a fluorescent indicator dye;
(b) said LAMP reaction mixture further comprises at least one detectable compound capable of intercalating into a double strand DNA, thereby providing a detectable signal upon production of at least one amplification product; and
(c) said sample is at least one of a biological sample and an environmental sample.

59. The kit according to claim 57, wherein said at least one nucleic acid sequence of interest is at least one nucleic acid sequence of at least one pathogen; optionally, at least one of:
(a) said pathogen is a viral pathogen;
(b) said pathogen is a viral pathogen and wherein said viral pathogen is at least one corona virus (CoV); and
(c) said pathogen is a viral pathogen, said viral pathogen is CoV and wherein said CoV is SARS CoV-2.

60. The kit according to claim 57, wherein at least one of:
(a) said kit is adapted for detection and monitoring of said at least one pathogen in at least one sample;
(b) said kit is adapted for the diagnosis and/or monitoring of at least one infectious disease caused by said pathogen in a mammalian subject; and
(c) said kit is adapted for the diagnosis and/or monitoring of COVID-19 in a mammalian subject.


SEQUENCE LISTING (20 sequences)

SEQ ID NO: 1 - DNA, 22 bp, Artificial Sequence
  E_Sarbeco_R2 primer
atattgcagcagtacgcacaca

SEQ ID NO: 2 - DNA, 26 bp, Artificial Sequence
  E_Sarbeco_P1 primer
acactagccatccttactgcgcttcg

SEQ ID NO: 3 - DNA, 26 bp, Artificial Sequence
  E_Sarbeco_F1 primer
acaggtacgttaatagttaatagcgt

SEQ ID NO: 4 - DNA, 23 bp, Artificial Sequence
  Primer FW IC (Upstream/1/Fw)
catgggaagcaagggaactaatg

SEQ ID NO: 5 - DNA, 22 bp, Artificial Sequence
  Primer RV IC (Downstream/2/Rv)
cccagcgagcaatacagaattt

SEQ ID NO: 6 - DNA, 26 bp, Artificial Sequence
  Probe IC
tcttccctcgaacctgcaccatcaat

SEQ ID NO: 7 - DNA, 19 bp, Artificial Sequence
  GeneN-A-F3
tggctactaccgaagagct

SEQ ID NO: 8 - DNA, 20 bp, Artificial Sequence
  GeneN-A-B3
tgcagcattgttagcaggat

SEQ ID NO: 9 - DNA, 25 bp, Artificial Sequence
  GeneN-A-LF (Loop Forward)
ggactgagatctttcattttaccgt

SEQ ID NO: 10 - DNA, 21 bp, Artificial Sequence
  GeneN-A-LB (Loop Backward)
actgagggagccttgaataca

SEQ ID NO: 11 - DNA, 41 bp, Artificial Sequence
  GeneN-A-FIP (Forward Inner Primer)
tctggcccagttcctaggtagtccagacgaattcgtggtgg

SEQ ID NO: 12 - DNA, 40 bp, Artificial Sequence
  GeneN-A-BIP (Backward Inner Primer)
agacggcatcatatgggttgcacgggtgccaatgtgatct

SEQ ID NO: 13 - DNA, 17 bp, Artificial Sequence
  RNaseP POP7 F3
ttgatgagctggagcca

SEQ ID NO: 14 - DNA, 18 bp, Artificial Sequence
  RNaseP POP7 B3
caccctcaatgcagagtc

SEQ ID NO: 15 - DNA, 20 bp, Artificial Sequence
  RNaseP POP7 LF
atgtggatggctgagttgtt

SEQ ID NO: 16 - DNA, 20 bp, Artificial Sequence
  RNaseP POP7 LB
catgctgagtactggacctc

SEQ ID NO: 17 - DNA, 41 bp, Artificial Sequence
  RNaseP POP7 FIP
gtgtgaccctgaagactcggttttagccactgactcggatc

SEQ ID NO: 18 - DNA, 45 bp, Artificial Sequence
  RNaseP POP7 BIP
cctccgtgatatggctcttcgtttttttcttacatggctctggtc

SEQ ID NO: 19 - DNA, 29903 bp, Severe acute respiratory syndrome coronavirus 2
  misc_feature: Complete genome of SARS-Cov2, NC_045512.2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 20 - protein, 1273 aa, Severe acute respiratory syndrome coronavirus 2
  MISC_FEATURE: Spike protein of SARS-Cov2, YP_009724390.1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT